(19) United States
(12) Patent Application Publication
(10) Pub. No.: US 2025/0161238 A1
(43) Pub. Date: May 22, 2025

(54) NMDAR Antagonists Prevent Ageing and Aging-Associated Conditions and Diseases Through Increasing 20S Proteasome Activity

(71) Applicant: Fikret Sahin, Ankara (TR)

(72) Inventor: Fikret Sahin, Ankara (TR)

(21) Appl. No.: 18/836,866

(22) PCT Filed: Feb. 8, 2022
(86) PCT No.: PCT/TR2022/050106

§ 371 Date: Aug. 8, 2024

(51) Int. Cl. A61K 31/13; A61K 9/00; A61P 25/28

(52) U.S. Cl. CPC A61K 31/13; A61K 9/0053; A61P 25/28

(57) ABSTRACT

The usage of NMDAR antagonists provides compositions and methods for treatment for the malfunctioning of the protein homeostasis network and interferes with crucial signaling pathways and is often associated with multiple human diseases. Importantly, the present invention provides informations comprising at least one NMDA receptor blockers for use in preventing and/or treating ageing and ageing associated conditions and diseases through increasing 20S proteasome activity.

The usage of NMDAR antagonists provides compositions and methods for treatment for the malfunctioning of the protein homeostasis network and interferes with crucial signaling pathways and is often associated with multiple human diseases. Importantly, the present invention provides informations comprising at least one NMDA receptor blockers for use in preventing and/or treating ageing and ageing associated conditions and diseases through increasing 20S proteasome activity.

### BACKGROUND

Aging is associated with malfunctioning of the protein homeostasis network and interferes with crucial signaling pathways and is often associated with multiple human diseases. However, it has been difficult to separate the effects of aging per se from those of age-associated diseases (Vilchez et al 2014).

In the last few decades, there has been a dramatic increase in group of medical conditions, collectively called “protein misfolding disorders” that include Alzheimer's disease (AD) and type 2 diabetes (Dobson, 2017). These protein-misfolding diseases disorders—almost unknown a century ago—are now becoming frighteningly common (Chiti and Dobson, 2017). Many of these “modern diseases” can be attributed to the fact that we are now living to ages unprecedented in all of human history, and as we get older, our protective mechanisms have a greater risk of failing (Hipp etal. 2014).

It is rapidly becoming evident that the protein misfolding disorders has huge implications not only for individuals and their relatives but also for society as a whole.

Moreover, the financial burdens of these conditions (because of the need for continuous care of patients, often for years) are reaching levels that no health care system can ignore. In the United States, for example, the costs of the AD alone are estimated to be $200 billion in 2015 and are predicted to exceed $1 trillion by 2050: in the next 35 years. therefore. the cost of care for patients with AD is anticipated to be $20 trillion in the United States alone (Alzheimer's Association—https://www.azm.org).

More than 30% of newly synthesized proteins are misfolded because of errors in translation or post-translational processes (Chiti and Dobson, 2017). The oxidative stress is another factor that cause protein unfolding. Upon oxidative damage, proteins unfold and expose hydrophobic regions which makes them prone to aggregation.

The contribution of oxidative stress in age-related organ changes is generally agreed that increases in reactive oxygen species (ROS) accompany aging, leading to functional alterations, increased incidence of disease, and a reduction in life span (Kregel and Zhang, 2007; Jung and Grune, 2013). Aerobic organisms regularly encounter ROS. Cells constantly generate oxidants and produce antioxidants. Despite their attempts to strike a healthy balance, organisms encounter numerous situations in which oxidant levels are no longer in sync with the cell's detoxification systems, generating a potentially lethal condition, termed oxidative stress.

A large body of literature exists that on increased amounts of oxidized proteins in skeletal muscle, heart, skin, liver, lymphocytes of various animal species (Jung and Grune, 2013). Oxidative stress has also been demonstrated to have a major role in the initiation and progression of cardiovascular diseases including atherosclerosis and hypertansion, cerebrovascular disease (stroke), type 2 diabetes, cancer, several chronic neurodegenerative diseases including AD and Parkinson's disease, osteoarthritis, osteoprosis, hearing loss, Age-related macular degeneration and others. Heart failure is the number one cause of hospitalization for people aged 65 and older, and nearly all patients with, or at risk of, heart disease have enhanced levels of oxidative stress ((Jung and Grune, 2013; Jung et al 2014).

According to the generally accepted opinion, misfolded and aggregated proteins caused by oxidative stress are key pathological findings even the natural aging process (Reichmann, et al., 2018).

Accumulation of ROS has long been considered to be a major contributor to aging and age-related diseases and the fact that aging tissues show increased oxidative damage remains undisputable.

Proteins are one of the major cellular targets of ROS. This is in large part due to the sheer number of oxidation-sensitive amino acid side chains present in proteins. The most frequently observed oxidative modifications involve cysteine and methionine, which have very high reaction rates with hydroxyl radicals or HOCl. In addition to the mostly reversible thiol and methionine oxidation reactions, which are often used to redox-regulate protein activity, proteins tend to undergo a number of irreversible side chain modifications, including thiol overoxidation, carbonylation and di-tyrosine formation. Newly synthesized polypeptide chains are particularly vulnerable, presumably because side chain modifications directly impact nascent protein folding. Nonetheless, mature proteins are sensitive as well. ROS react with residues during local unfolding events, shifting the equilibrium of proteins towards unfolding and aggregation. Hydroxyl radicals are considered to be one of the most reactive ROS and are responsible for much of the oxidative damage that proteins, lipids and DNA experience once ROS levels increase. Extensively cross-linked and hydrophobically bonded protein aggregates bind to other oxidation products of lipids, carbohydrates, and nucleic acids and tend to accumulate within cells producing such entities as inclusion bodies, Heinz bodies, ceroid pigments, ‘age’ pigments, and lipofuscin. large aggregates, including amyloid, amorphous, or native-like assemblies, have links with a number of human diseases and physiological processes including ageing and ageing related diseases (Reichmann, et al., 2018).

A range of proteins involved in these processes (e.g., transthyretin, insulin, β-2-microglobulin, superoxide dismutase) are natively folded. However the vast majority of them involved in protein-misfolding disorders are particularly those that are relatively small, are intrinsically disordered protein (IDPs). Disorder is mostly found in intrinsically disordered regions (IDRs) within an otherwise well-structured protein. The term IDP therefore includes proteins that contain IDRs as well as fully disordered proteins. Intrinsically disordered systems can also be generated following proteolysis from larger proteins that are otherwise folded, such as the amyloid-β peptide and the amyloidogenic fragment of gelsolin. Because of their lack of stable 3D structure, IDPs are extremely sensitive to their environment, much more so than ordered proteins. Also, this lack of fixed structure is often associated with extreme binding promiscuity of IDPs (Breydo et al., 2017)

Illustrative examples of amyloidogenic IDPs important to human diseases are listed in Table 1. The best known examples of amyloidogenic IDPs include amyloid β (Aβ) peptides, tau (AD), α-synuclein (Parkinson's disease (PD) and other synucleinopathies), TAR DNA-binding protein 43 (TDP-43), fused in sarcoma (FUS) protein (amyotrophic lateral sclerosis (ALS)), and huntingtin (Huntington's disease). A number of human functional amyloids (cytoplasmic polyadenylation element-binding protein (CPEB), T-cell-restricted intracellular antigen-1 (TIA-1), premelanosome protein 17 (Pmel17), secretory peptide hormones), and cell cycle related proteins including p53, p27, p21 (cell cycle, apoptosis).

Twenty percent of cellular proteins are classified as IDPs and as many as 41% of the eukaryotic proteome is predicted to contain IDRs. There are no evident similarities among the proteins in sequence, structure, or function.

The different conformational states adopted by proteins involve a highly complex series of equilibria whose thermodynamics and kinetics in a normally functioning living system are determined by their intrinsic amino acid sequences as well as through degradation processes, and other sophisticated quality control mechanisms.

The protein quality control in cell is maintained by the protein homeostasis (proteostasis) network, which consists of the protein synthesis, protein folding complexes, and protein degredation machinery systems.

Proteome imbalance is accompanied by widespread protein aggregation, with abundant proteins that exceed solubility contributing most to aggregate load.

(Walther etal. 2017)

Therefore, clearance of misfolded proteins is critical for cell survival because misfolding alters a protein's three-dimensional structure, impairing its biological activities and increasing its propensity to form toxic aggregates (Hartl, 2017).

Modulation of protein concentration via regulation of the proteolytic machineries has long been validated as promising milieu for the development of treatments for ageing and ageing related different human diseases such as neurodegeneration, cancer, and autoimmunity, ageing and ageing related diseases.

Therefore, increasing proteolytic capacity provides an important approach to maintaining proteostasis (Njomen and Tepe, 2019)

A major pathway for the proteolytic machineries in the cell is the proteasome system. Most enzymatic protein breakdown was initially assumed to take place in the relatively harsh acidic environment of the lysosome. However, in the 1970s Goldberg et al. demonstrated that cells lacking lysosomes were still capable of protein turnover via an autonomous ATP-stimulated proteolytic pathway. Both a 20S and a 26S proteasome was discovered, named according to their Svedberg sedimentation coefficients (a reflection of their relative molecular mass), and were thought to represent two separate entities within the cell until it was demonstrated that the 26S proteasome consisted of two 19S regulators bound to a 20S core (Hipp et all. 2014).

Although there is evidence suggesting that chaperone mediated autophagy is activated during oxidative stress response, the proteasome represents the major proteolytic machinery for the removal of oxidized and misfolded proteins (Njomen and Tepe, 2019 Hipp et all. 2014, Pohl and Dikic, 2019).

The proteasome is a central protein degradation machine in eukaryotes. Through hydrolysis activities, it removes damaged proteins and ensures the delivery of amino acids to support on going biosynthesis. The proteasome makes up a staggering 1%-2% of the entire proteome in healthy cells.

Degradation by the Proteasome Falls into Two Major Categories:

The ubiquitin-proteasome system (UPS) and the ubiquitin-independent proteasome system (UIPS) (Njomen and Tepe, 2019).

At the center of the UPS is the proteasome as described by Yu and Matouschek (Yu and Matouschek, 2017). The human 26S proteasome consist of a barrel-shaped 20S core particle (CP) capped by one or two 19S regulatory particles (RPs) also called PA700. The 20S CP is a threonine protease that consists of four stacked rings. The two inner β-rings contain three catalytic subunits (β5, β2, and β1) that display chymotrypsin-like, trypsin-like, and caspase-like activity, respectively. The outer α-rings serve as gated channels that regulate substrate entry and product exit from the inner catalytic chambers. These outer rings also act as docking surfaces for the 19S RPs. The α and β-rings are each composed of heteroheptameric subunits; α1-α7 and β1-β7, respectively. The 26S proteasome is formed when the 28-subunit CP is docked on one or both ends by the ATPase 19S cap (PA700). The 19S RP is responsible for 20S gate opening, substrate recognition and binding, unfolding, and threading of ubiquitinated substrates into the 20S CP (Yu and Matouschek, 2017; Jung etal. 2014)

A number of other non-ATPase regulatory particles such as the 11S complex (PA28) and PA200 also reversibly associate with the 20S CP by docking onto the α-rings. The 19S RP consist of two subcomplexes, the base that interacts directly with the 20S and a peripheral lid. The base is comprised of hexameric AAA-ATPase subunits, Rptl-Rpt6, and tetrameric non-ATPase subunits, Rpn1, Rpn2, Rpn10, and Rpn13. The ATPase activity in the base is essential for protein substrate unfolding, gate opening, and translocation of substrate into the 20S core. The lid is made of nine non-ATPase subunits; Rpn3, Rpn5-Rpn9, Rpn11, Rpn12, and Sem1. The lid, specifically the Rpn11 subunit, functions as a deubiquitinase. Rpn13, Rpn10, and other reversibly associated proteins, such as radiation sensitivity abnormal 23 (Rad23) and dual-specificity protein kinase 2 serve as ubiquitin receptors that direct polyubiquitinated proteins to the proteasome. The Rpn subunits also create docking site (s) for other proteins including the proteasome associated deubiquitinating enzymes, ubiquitin specific peptidase 14 (USP14), and ubiquitin C-terminal hydrolase (UCH37) (Collins and Goldberg, 2017; Yu and Matouschek, 2017).

### The 26S Proteasome and Ubiquitin-ATP-Dependent Proteolysis.

The 26S proteasome mainly targets structured proteins for degradation, although a certain fraction of misfolded, IDPs and proteins containing IDRs are also degraded by the 26S. Structured proteins due for degradation are tagged with chains of polyubiquitin which serve as a degron for their turnover. Polyubiquitinated proteins are recognized by the ubiquitin receptors, Rpn10 and Rpn13. The ubiquitin tag is then removed by deubiquitinases such as the Rpn11, USP14, and UCH37. The ATPase activities of the 19S base then unfolds and directs the protein into the catalytic chamber for degradation (Collins and Goldberg, 2017).

Ubiquitin is a small protein (˜8 kDa) of 76 amino-acids, consisting of seven lysine (K) residues at positions 6, 11, 27, 29, 33, 48, and 63 through which it can be attached to protein substrates. A well-defined series of enzymes, ubiquitin ligases (E1, E2, and E3) coordinate the attachment of mono- and polyubiquitin to proteins. Ubiquitin is first activated in an ATP-dependent reaction by an E1 ubiquitin-activating enzyme, to which it becomes attached by a thioester bond. Subsequently, the activated ubiquitin is transferred to the active site cysteine of the E2 ubiquitin-conjugating enzyme. Ubiquitin-protein ligase (E3), together with E2 catalyze the transfer of ubiquitin onto the protein that is destined for degradation (Collins and Goldberg, 2017; Yu and Matouschek, 2017)

### The 20S Proteasome and Ubiquitin-Independent Proteolysis.

Unlike the 26S proteasome which primarily degrades polyubiquitinated proteins, the 20S directly degrades misfolded, oxidatively damaged, and IDPs and IDR-containing proteins and does not require the unfoldase activity of the 19S base. Furthermore, 20S-mediated proteolysis does not require polyubiquitination of its substrates. IDPs and IDR-containing proteins, which lack this 3-dimensional structure, are thought to readily traverse the a-ring gate of 20S proteosome. Twenty percent of cellular proteins are classified as IDPs and as many as 41% of the eukaryotic proteome is predicted to contain IDRs, suggesting that the substrate pool of the UIPS may be considerably large. These substrates are particularly relevant because they include the proteins that accumulate in neurodegenerative disorders, such as amyloid beta, tau, TDP-43, and a-synuclein, Prion Protein (PrP), Polyglutamine Repeats.

In cells, IDPs typically have shorter half-lives relative to structured proteins. The UPS and UIPS have both been shown to facilitate the rapid proteasomal degradation of IDPs, such as p21, p53 and p27 (Chiti and Dobson, 2017).

Although the degradation of oxidatively damaged proteins can occur by both ubiquitin/ATP-dependent (i.e. 26S-dependent) and ubiquitin/ATP-independent (i.e. 20S-dependent) mechanisms, various studies have implied that 20S proteasome is more critical for the removal of damaged proteins (Jung and Grune 2008; Jung et al. 2014)

It has been shown that 20S proteasomes can degrade oxidized proteins (e.g. histones, hemoglobin, superoxide dismutase) in vitro, independent of ubiquitin/ATP. This phenomenon has been attributed to 20S proteasome recognition of, and interaction with, abnormally exposed hydrophobic patches in oxidatively damaged and unfolded proteins that induce conformational changes in the 20S structure and promote channel opening followed by protein degradation (Davies, 2001, Ishii et al. 2015)

Therefore, the 20S proteasome complex is capable of directly targeting oxidatively damaged proteins to detoxify the cell.

This may be in part because of the fact that the 20S proteasome is more resistant to oxidative stress than the 26S proteasome as the 20S complex can maintain activity even upon treatment with moderate to high concentrations of H₂O₂, whereas the 26S proteasome is much more vulnerable likely because of the observed separation of the 19S particle from the 20S core in the presence of H₂O₂(Ishii et al. 2005; Davies, 2001). Upon incubation with the metal-catalyzed ROS generating system (Cu2+/H₂O₂), 26 S proteasome was converted to lower molecular weight protein species in a Cu2+ concentration-dependent manner, whereas the protein band corresponding to 20 S proteasome was virtually unchanged, suggesting that changes in 26 S proteasome might be due to the oxidative modification of 19 S regulatory subunit(s) (Jung and Grune, 2013; Jung at al. 2014; Jung at al. 2014).

The 20S proteasome plays a pivotal role in the selective recognition and degradation of oxidized proteins, being responsible for the degradation of approximately 90% of all intracellular oxidation-damaged proteins. Notably, degradation by the 20S proteasome occurs in a Ub-independent manner. The major recognition motifs of the substrates seem to be hydrophobic patches exposed on the surface of the damaged protein. Studies indicate that the cell is able to reversibly disassemble 26S proteasomes to elevate the levels of free 20S particles under oxidative conditions or in response to mitochondrial dysfunction (Davies, 2001, Aiken etal. 2011).

The most important concern that may occur as consequence of decreased protesome activity is the accumulation of potentially toxic un/misfolded proteins as well as protein aggregates. The proteasome impairment will eventually affect the vital function of cytosolic processes and all other organelles due to the accumulation of unfolded and damaged proteins. In addition, the accumulating waste will also impede the recycling of amino acids and Ub that are both required for cell survival (Reichmann, etal. 2018).

The accumulation of unfolded, misfolded, or damaged proteins severely impairs the function of organelles and cells and has been recognized as a crucial factor in aging and a wide variety of diseases.

Several reports indicate that one of the first detectable consequences of proteasome dysfunction concerns mitochondria, which develop deleterious alterations in their proteome. Mitochondria are the cellular power plants that produce energy through oxidative phosphorylation. They also directly contact most (if not all) other cytosolic organelles (e.g., endoplasmic reticulum (ER), plasma membrane, and peroxisomes) to generate specialized networks that control several cellular functions like Ca2+ homeostasis, lipid synthesis, and apoptosis. Importantly, dysfunctional mitochondria are also the major source for oxidative stress through aberrant production of ROS, which has profound implications for the pathogenesis of various diseases, in particular neurodegenerative disorders and cancer, as well as aging.

As an inactive proteasome also compromises the ER-associated degradation (ERAD) pathway, severe ER stress is another unavoidable consequence of sustained proteasome inhibition (Ding etal, 2007).

The protesome pathway preserve mitochondrial plasticity and quality by controlling several levels of mitochondrial dynamics. Depending on the degree of stress, they trigger adaptive responses (moderate stress) and removal of the damaged organelle by mitophagy (sustained stress) or, if these measures fail, induce cell death (irreparable damage). These activities and processes are tightly interconnected. During moderate stress, as a first line of defense against mitochondrial dysfunction, the protesome pathway is in charge of outer membrane PQC (protein quality control) and the degradation of damaged proteins in a process called outer mitochondrial membrane-associated degradation (OMMAD). Moreover, the protesome pathway directly controls mitochondrial dynamics by ubiquitinating and degrading proteins involved in mitochondrial fusion and fission processes (Roussel etal., 2013).

While the 20S proteasome has largely been considered a dormant protease that is only activated upon binding to regulatory complexes, significant and increasing evidence has clearly demonstrated that the 20S core has a major a role in overall protein degradation, independent of ubiquitin and ATP (Raynes et al 2016). The 20S proteasome is the core unit of all proteasomal systems and (in mammals) constitutes approximately 1% of total cellular protein (Raynes et al 2016). The 20S proteasome is most abundant throughout the cytoplasm and the nucleus. In addition, it has been found to be directly attached to various organelle membranes, particularly the endoplasmic reticulum, where it degrades membrane-bound substrates (Hirsch and Ploegh, 2000).

Therefore, it is agreed that the proteasome is the cell's first defense mechanism against accumulating proteotoxic stresses induced by oxidative damage (Njomen and Tepe, 2019; Jones and Tepe, 2019, Korovila, etal, 2017).

### Roles of the Proteasome in Ageing

The accumulation of unfolded, misfolded, or damaged proteins severely impairs the function of organelles and cells and has been recognized as a crucial factor in aging and a wide variety of diseases.

During ageing, oxidized proteins appear to aggregate and accumulate to abnormally high levels. Such large aggregates, including amyloid, amorphous, or native-like assemblies, have links with a number of human diseases and physiological processes including ageing and ageing related diseases (Chondrogianni et al., 2015).

It is well known that total rates of protein degradation are reduced as an organism ages. It is also known that a decline in proteasome activity has been broadly implicated in ageing and age associated diseases, including neurodegeneration. Presumably this decline contributes to a catastrophic imbalance in proteostasis and accumulation of damaged and/or misfolded proteins.

The species with increased longevity and long-lived individuals within a given species exhibit higher proteasome activity and are less susceptible to diseases, including neurodegeneration (Chondrogianni et al, 2015).

Multiple studies have found that increased proteasome activity is beneficial, decreasing the accumulation of pathogenic proteins and delaying aging. For example, delivery of purified 20S to cells through direct injection has been shown to accelerate clearance of tau. Identified small molecules that inactivate the deubiquitinating activity of USP14, the deubiquitinating enzyme, allosterically activate proteasomal degradation of poly Ub-conjugated proteins and, consistent with the model, accelerate turnover of tau in vitro (Lee et all. 2010; Boselli, et all. 2017).

In addition, a study using human lung fibroblast WI-38 cells as a model for replicative senescence found that activity were mitigated in senescent cells, correlating with an increase in carbonylated proteins. In addition, blocking the proteolytic activity of the 20S core in low passage WI-38 cells induced an aged-phenotype. In an in vivo study using rat liver tissue, 20S proteasome peptidylglutamyl peptide hydrolyzing activity (caspase like) showed a 60% reduction in aged rats compared to young animals. Enhancing proteasome activity reduces the proteotoxic burden cells experience upon aging. In humans, rodents, and cells, increased proteasome activity delays aging and results in longer lifespan by reducing proteotoxic pathologies. Supporting this idea, cells from human centenarians exhibit enhanced proteasome activity compared to cells from adults of different ages (Raynes, et all., 2016)

While activity of the 26S proteasome and ubiquitin-dependent proteolysis does appear to increase in a tissue-specific manner during aging, the ability to degrade oxidized proteins by the 20S proteasome does indeed decrease with age, thereby contributing to the overall decline in protein homeostasis.

Aged cells contain a latent pool of free (eg, unbound) 20S. The relative levels of 20S and PA-bound 20S change with ageing and disease. Using label-free proteomics in 9 different human cell lines, it was shown that 21%-35% of the total proteasome pool is PA700-bound (26S) and less than 10% is bound to UIPS-specific PAs, while there maining 66% is unbound 20S. This suggests that cells may contain a latent pool of 20S that is not bound to any PA. Because PAs enhance the rate of proteolysis by as much as 20-fold, these findings suggest that some cells have a pool of 20S that is poised to be activated.

In aged individuals, who are the major victim of this disease class, the proteasome exists mainly as the latent 20S, thus making 20S a better target for these diseases (Pickering and Davies, 2012)

Since oxidatively modified proteins are known to be more susceptible to proteolytic degradation by the proteasome than native proteins, it seems obvious that the proteolytic activity of the proteasome is required more and more with increasing age. In contrast, it was known that the proteasome activity is progressively decreased with increasing age particularly by accumulating oxidized and cross-linked cellular proteins as shown in experiments. Therefore, the age-related accumulation of oxidized proteins may result from either an increase in protein oxidation or a decline in the degradation of oxidized proteins.

In any case, attempts to improve proteostasis pharmacologically would probably have to occur at an early stage of disease before the manifestation of severe cellular dysfunction (Hipp etal. 2014). Therefore, timely removal of oxidatively damaged proteins is of critical importance to maintain normal cellular homeostasis and viability.

Protein oxidation is a factor in multiple diseases of aging, including Alzheimer's and Parkinson's disease, atherosclerosis and arteriosclerosis, stroke, cancer, arthritis, cataract, macular degeneration, frailty and many others. Loss of protein homeostasis and the adaptive capacity to respond to oxidative stress during aging may have a staggering impact on the world economy.

Therefore, finding a new method that especially increase the 20S activity will be invaluable.

L-glutamate is the major excitatory neurotransmitter in the central nervous system and acts on the NMDA receptor. The ionotropic NMDA receptor, which fluxes both calcium and sodium, is located on the neuronal cell surface and has multiple binding sites (i.e., glycine, polyamine, NMDA) as well as an ion-channel which has several internal binding sites (i.e., Mg++, PCP) (Lucia et al. 2019). Unique properties of this receptor include: Voltage-dependency, a high permeability to Ca++, a requirement for coactivation by glycine, and blockade by physiological concentrations of Mg++.

Recently, increasingly convincing evidence has demonstrated that specific glutamate receptors and glutamate transporters were also expressed in non-neurological tissues, such as the lung, liver, kidney, stomach and immune system, which suggested that glutamate plays an important role in the regulation of physiological function in various peripheral organs. It has also been found that glutamate exhibits different effects in non-neurological diseases, such as renal damage including ischaemia, toxic injury and renal cell carcinoma liver inflammation, myocardial ischaemia, numerous types of lung diseases, including lung oedema, lung fibrosis and lung cancer, gastric ulcer, outoimmunity related to the innate immune system and adoptive immune systems and others (Du etall. 2016).

Glutamate receptors, key component of the glutamate pathway, exhibit their role as signal detectors and transmitters. Glutamate receptors are mainly subdivided into two groups; ionotropic glutamate receptors (iGluRs) that constitute ion channels, and metabotropic glutamate receptors (mGluRs) which are members of the G protein-coupled receptor (GPCR) superfamily.

N-methyl-D-aspartate (NMDA) receptors belong to the iGluRs family comprising Kainate receptors, (2-amino-3(3-hydroxy-5-methylisoxazol-4-yl)propanoic acid) AMPA and (N-methyl-D-aspartate receptor) NMDA receptors (Paoletti et all. 2013, Petit-Pedrol and Groc, 2021, Vieira et all. 2020; Groc et all. 2009)

Unlike other iGluRs, NMDA receptors are well-known for their high Ca2+ ion conductance, voltage dependent-channel blockade, and the compulsory binding of two endogenous ligands for receptor activation. Notwithstanding the tight regulation of glutamate synaptic concentration, excessive glutamatergic signaling is a key feature of neurodegenerative pathologies resulting in drastically elevated intracellular Ca2+ ions levels and subsequent neuronal apoptosis. Given the relatively high calcium permeability, NMDA receptors are predominantly implicated in these deleterious events, collectively known as excitotoxicity.

NMDA receptors are plastic heterotetrameric architectural complexes residing either at synaptic or extrasynaptic membranes (such as cell body and the dendritic shaft). Subunits belong either to the GluN1, GluN2, or GluN3 classes. Specifically, eight isoforms of GluN1 exist (a-h, different splice variants of one single gene), four GluN2 (A-D, encoded by four different genes) and two GluN3 (A-B, encoded by two distinct genes) subunits have been identified. Functional receptors entail two GluN1 subunits and a GluN2/GluN3 subunit combination (triheteromeric assembly).

The activation of NMDA receptors relies on the combination of two agonists, that are glutamate and glycine or D-serine. Usually, NMDA receptors consist of two glycine binding GluN1 subunits and two glutamate-binding GluN2 subunits. There are multiple ligand-binding sites on the NMDA receptors including glutamate binding sites, glycine binding sites, ion channels pore, and allosteric binding sites on the aminoterminal domain (ATD), which modulate receptor activity in a subtype-selective manner. More and more studies have shown that different subtypes of NMDA receptors generate different functional outputs

NMDA receptor antagonist’ or “N-methyl D-aspartate receptor antagonist’ relates to compounds which are in vivo and/or in vitro capable to block, either completely or partially, the action and/or function of the NMDA receptor or the NMDA receptor complex. Several glutamate receptor antagonists (NMDA receptor antagonists) and their neuroprotective roles have been described.

There are three classes of NMDAR antagonists: competitive antagonists, uncompetitive channel blockers, and non-competitive allosteric inhibitors. Although ultimately resulting in a similar outcome, each antagonist class has a distinct mechanism of action. Competitive NMDAR antagonists act by occupying the glycine or glutamate-binding site located on either the GluN1 or GluN2 subunit, respectively. This class of antagonist prevents endogenous ligand binding, thereby preventing activation of the receptor. Competitive and uncompetitive antagonists tend to be poorly selective for a specific GluN1/GluN2 subunit, whereas noncompetitive allosteric inhibitors display much higher selectivity. Uncompetitive channel blockers act by occluding the NMDAR ion channel pore (Das 2020; Omar et all. 2020).

Uncompetitive channel blockers of NMDAR are typically positively charged and require depolarization, such that the Mg2+ block is removed and the binding site exposed. Once bound, these channel blockers can prevent the flow of ions in/out of the channel thus producing inhibition. It is important to note that when channel blockers are in the pore, glutamate may still bound to the NMDAR potentially resulting in Ca2+-independent intracellular signaling; though the significance of this phenomena is not fully understood. Finally, non-competitive allosteric modulators inhibit activity of NMDARs by acting at the N-terminal or “agonist binding” domains of the GluN1/GluN2 subunits to reduce the activity of the receptor by decreased binding of endogenous ligands.

The class of uncompetitive ion channel blockers are ketamine, dizocilpine (MK-801), and memantine (Das 2020; Omar et al. 2020).

Various NMDA receptor antagonists have been developed. Some NMDA (receptor/complex) antagonists block the ion channel, others act at the glycine site. Other substances are selective for NR2B NMDA receptor subtypes. Non limiting examples of agents that can function as antagonists are described and patented. NMDA receptor antagonists are described in detail for NMDA receptors and receptor specific antagonist, formula, additional activities, for example, futerman et al. U.S. Pat. Nos US 2017/0273917 A1. Recently, Ahmed et all. has described patented NMDA receptor antagonists (Ahmed et al. 2020)

Memantine (1-amino-3,5-dimethyl adamantine) is a systemically-active uncompetitive NMDA receptor antagonist having moderate affinity for the receptor and strong voltage dependency and rapid blocking/unblocking kinetics. Memantine has been shown to be useful in alleviation of various progressive neurodegenerative disorders such as dementia in patients with moderate to severe Alzheimer's disease, Parkinson's disease, and spasticity (see, e.g., U.S. Pat. Nos. 5,061,703; 5,614,560, and 6,034,134). Memantine is approved for the treatment of moderate to severe Alzheimer's disease by the FDA and the EMA. Increasing evidences show that Memantine acts in the benefit of cancer, cardiovascular and renal disorders in addition to neurological disorders (Shafiei-Irannejad et al. 2021; Cauli et al. 2014).

Memantine has multiple mechanisms of action that are hypothesized to produce its safety and efficacy profile. These include at least: (1) use-dependent channel blocking or the binding and blocking of agonist gated open channels more rapidly than closed channels, (2) low binding affinity or faster effective blocking rates, (3) rapid intrinsic association kinetics, (4) rapid dissociation kinetics and Voltage-dependency which allow blocking during synaptic depolarization but allows physiologic neuronal activity, (5) NMDA subunit selectivity in which memantine preferentially blocks the NR2C and NR2D subunits and to a lesser degree the NR2B Subunit, (6) partial trapping or the mechanism where a fraction of the blocker can escape from the closed channel, (7) multiple actions at the NMDA receptor or allosteric non competitive actions, (8) and actions at other receptor targets.

In humans, doses of 20 mg per day of memantine produce serum levels which range from 0.5-1.0 uM. However, 30 mg/kg dose of memantine is well tolerated in mice (see Futerman et al. U.S. Pat. Nos US 2017/0273917 A1). Brain microdialysis with in vivo recovery indicate that free rat brain concentrations are 20-30% lower in plasma, whereas CSF sampling in human Subjects showed 30-40% lower concentrations. There was no adverse drug interaction when memantine was combined with AchE (acetylcholinesterase inhibitors). No induction of HSP (heat shock protein) or neuronal vacuolization and necrosis have been observed in animals studies.

At levels of 1-10 uM, the mechanism of action of memantine is specific for antagonism of the NMDA receptor and does not affect other ligand-gated or Voltage gated channels. Importantly, these concentrations (6-10 uM) do not attenuate LTP in hippocampal slices or alter the function of the postsynaptic excitatory currents.

Memantine preferentially blocks the NR2C and NR2D Subunits, has intermediate potency at NR1A/2B and weak effects at NR2A of the NMDA receptor.

Memantine gains rapid access to the open channel at the NMDA receptor at the initiation of pathological overactivity and thereby attenuates its progression. Its high index of therapeutic efficacy and safety is due to the ability to block tonic low level pathological activation of NMDA receptors by agonists and mild membrane depolarization in chronic neurodegeneration diseases while simultaneously allowing physiological NMDA activation following synaptic release of glutamate.

### Protesome Activity Measurement Assay

Traditionally, an increase in the ubiquitin levels measured in total cell lysates has been used as an indicator of declining proteasome function. Given that ubiquitinylation plays roles in cell signaling independent of proteasomal degradation, assessing changes in ubiquitin levels may be a deceptive indicator of proteasome activity (Gan et al. 2019).

The identification of compounds that increase proteasome activity is hindered by the lack of good assays. To appropriately assess the effect of compounds on the UPS, solid methods are required that accurately determine the total amount of 26S proteasome in cells as well as its activity. A commonly used method to measure proteasome activity is to make use of fluorogenic Substrates. In this type of experiment small peptides are linked to a 7-Amino-4-methylcoumarin (AMC) group. Upon cleavage of this group by proteases such as the proteasome the AMC group becomes fluorescent and this signal can be measured overtime. When the proper controls are included, the speed of conversion can be derived from such data and interpreted as a measure of proteasome activity. The advantage of this method is that each of the catalytic subunits can be measured separately by using a substrate-AMC conjugate that is preferentially cleaved by one of the three catalytic subunits. One disadvantage is that only the activity of individual subunits can be measured, and not the total proteasome activity, as not all subunits may equally contribute to the total proteasome activity. Furthermore this type of experiment is almost always performed with cell lysates or purified proteasome. Data from this type of experiments may therefore not be relevant in more complex environments such as whole cells or the situation in vivo. Using cell permeable versions of fluorogenic Substrates can help to overcome some these limitations (Gan et al. 2019).

### Senolytic Compounds

### Cellular Senescence

Mammalian cells have the exceptional ability to adapt to perturbations in the extracellular and intracellular environments. Perturbations in a cell's microenvironment promote the activation of metabolic and molecular changes to ensure cell survival. Despite these adaptive mechanisms, chronic or severe, irreparable damage will terminate the damaged cell to preserve an organism's life. The termination of a damaged cell refers to the end of the normal physiological status of the cell. This can happen through cellular division inactivation, a state known as senescence.

Intra and extracellular signals that can contribute to cells' entering the senescent cell fate mainly include signals related to tissue or cellular damage and/or cancer development. These include DNA damage, telomeric uncapping or dysfunction, exposure to extracellular DNA, oncogene activation, replicative stress or inducers of proliferation (such as growth hormone/IGF-1), protein aggregates, misfolded proteins, failed protein removal through decreased proteasome activity, presence of AGEs due to the reaction of reducing sugars with amino groups in proteins (e.g. Haemoglobin A1c is an AGE), saturated lipids and other bioactive lipids (bradykines, certain prostaglandins, etc.), reactive metabolites (e.g. ROS, hypoxia or hyperoxia), mechanical stress (e.g. bone-on-bone stress in osteoarthritis or shear stress such as occurs on the venous side of AV fistulae for haemodialysis or around atherosclerotic plaques), inflammatory cytokines (e.g. TNFa), damage-associated molecular patterns (DAMPs, e.g. released intracellular contents signalling breakage of neighbouring cells), and pathogen-associated molecular patterns (PAMPs, e.g. bacterial endotoxins). These inducers active one or more senescence-promoting transcription factor cascades, in some cases involving p16INK4a-retinoblastoma protein (Rb), in others, p53 and p21CIP1, both of these pathways, or other pathways.

Therefore, when the damage is severe enough but is not lethal, cells switch to a permanent, non-proliferating state. This state is characterized by an inflammatory phenotype known as the senescence-associated secretory phenotype (SASP). The SASP consists of a myriad of cytokines, chemokines, growth factors, and proteases that initiate inflammation, wound healing, and growth responses in nearby cells. In young healthy tissues, the SASP is typically transient and tends to contribute to the preservation or restoration of tissue homeostasis. However, senescent cells increase with age, and a chronic SASP is known or suspected to be a key driver of many pathological hallmarks of aging, including chronic inflammation, tumorigenesis, and impaired stem cell renewal.

Data from several laboratories, strongly support the idea that senescent cells and the SASP drive multiple age-related phenotypes and pathologies, including atherosclerosis, osteoarthritis, cancer metastasis, cardiac dysfunction, myeloid skewing, kidney dysfunction, and overall decrements in health span (Saez-Atienzar and Masliah 2020).

Senescent cells can accumulate at aetiological sites of multiple diseases throughout the lifespan. For example, in humans, senescent cells accumulate in adipose tissue in diabetes and obesity, in the hippocampi and frontal cortex in Alzheimer's disease, the substantia nigra in Parkinson's disease, bone and marrow in age-related osteoporosis, lungs in idiopathic pulmonary fibrosis, liver in cirrhosis, retinae in macular degeneration, plaques in psoriasis, kidneys in diabetic kidney disease, endothelium in pre-eclampsia, and the heart and major arteries in cardiovascular disease, amongst many other conditions.

SASP is one of the three main features of senescent cells, the other two features being arrested cell growth, and resistance to apoptosis. SASP factors can include the anti-apoptotic protein Bcl-xL, but growth arrest and SASP production are independently regulated. Although SASP from senescent cells can kill neighboring normal cells, the apoptosis-resistance of senescent cells protects those cells from SASP.

Senescent cells are highly metabolically active, producing large amounts of SASP, which is why senescent cells consisting of only 2% or 3% of tissue cells can be a major cause of aging-associated diseases. SASP factors cause non-senescent cells to become senescent. SASP factors induce insulin resistance. SASP disrupts normal tissue function by producing chronic inflammation, induction of fibrosis and inhibition of stem cells. Chronic inflammation associated with aging has been termed inflammaging, although SASP may be only one of the possible causes of this condition. Chronic inflammation due to SASP can suppress immune system function, which is one reason elderly persons are more vulnerable to COVID-19.

SASP induces an unfolded protein response in the cell because of an accumulation of unfolded proteins, resulting in proteotoxic impairment of cell function.

Despite the fact that cellular senescence likely evolved as a means of protecting against cancer early in life, SASP promotes the development of late-life cancers. Cancer invasiveness is promoted primarily though the actions of the SASP factors metalloproteinase, chemokine, interleukin 6 (IL-6), and interleukin 8 (IL-8). In fact, SASP from senescent cells is associated with many aging-associated diseases, including not only cancer, but atherosclerosis and osteoarthritis. For this reason, senolytic therapy has been proposed as a generalized treatment for these and many other diseases.

Clearing already formed senescent cells, many of which harbour oncogenic mutations or cause cancer-promoting inflammation due to their SASP, prevents or delays cancer development. Indeed, senolytic drugs, agents that selectively eliminate senescent cells, delay development of multiple forms of cancer in old mice as well as cancer-prone (Atienzar and Masliah, 2020).

### Targeting Cellular Senescence

Ablation of senescent cells has been postulated as a promising therapeutic approach to target the ageing phenotype and, thus, to prevent, delay or mitigate ageing-related diseases. The aim with senolytic compounds is to rejuvenate organisms by selectively killing senescent cells and their efficacy is based on the ability of senescent cells to resist apoptosis. These cells exhibit upregulation of pro-survival pathways to protect themselves from the damaging (and pro-apoptotic) effect of the SASP. To date, six pro-survival pathways have been detected in senescent cells: the BCL-2-BCL-xL pathway, the MDM2-p53-p21Cip1-serpine elements pathway, ephins-dependence receptors-tyrosine kinases, the PI3K-AKT-ceramide metabolic pathway, the hypoxia inducible factor 1α (HIF-1α) pathway and the SP-90-dependent pathway. Senolytic compounds interfere with these pro-survival pathways to let senescent cells die by apoptosis (Atienzar and Masliah, 2020).

Therefore p53, p21, BCL-2, AKT, SP-90, pRb and others involved in these pathways are target of the Senolytic compounds.

### The Geroscience Hypothesis

The Geroscience Hypothesis posits that fundamental ageing mechanisms are ‘root cause’ contributors to the increasing burden of disorders and diseases with advancing age that are responsible for the bulk of morbidity, mortality and health costs in the developed and developing worlds. These fundamental ageing processes include: (1) chronic low grade ‘sterile’ (absence of bacteria, fungi, etc.) inflammation often accompanied by fibrosis, 2) macromolecular dysfunction (e.g. DNA damage, telomere uncapping, protein misfolding and aggregation, decreased proteasome activity, increased advanced oxidation-glycation end-products, lipotoxicity and accumulation of bioactive lipids) and organelle dysfunction (altered nuclear membranes related to deficient lamin B, mitochondrial dysfunction leading to reduced fatty acid metabolism, higher glucose utilization, depletion of NAD+ and increased ROS generation, etc.), 3) stem, progenitor and immune cell dysfunction (including altered proliferative capacity and dysdifferentiation with failure to develop into functional mature cells, declines in ‘geroprotective’ factors [e.g. a-Klotho], contributing to stem and progenitor cell dysfunction) and 4) cellular senescence. Unitary Theory of Fundamental Aging Processes hypothesizes that these fundamental ageing processes may be interlinked. Therefore, targeting any one fundamental ageing process (e.g. decreased proteasome activity, protein misfolding, cellular senescence) genetically or with drugs should affect many or perhaps all of the rest. Indeed, consistent with the Unitary Theory, senescent cells contribute to inflammation, fibrosis, DNA damage, development of protein aggregates, failed autophagy, lipotoxicity, mitochondrial dysfunction, depletion of NAD+, ROS generation and stem, progenitor and immune cell dysfunction (Austad, 2016).

### SUMMARY OF THE INVENTION

The accumulation of unfolded, misfolded, or damaged proteins severely impairs the function of organelles and cells and has been recognized as a crucial factor in aging and a wide variety of diseases.

During ageing, oxidized proteins appear to aggregate and accumulate to abnormally high levels.

Such large aggregates, including amyloid, amorphous, or native-like assemblies, have links with a number of human diseases and physiological processes including ageing and ageing related diseases.

It is well known that total rates of protein degradation are reduced as an organism ages. It is also known that a decline in proteasome activity has been broadly implicated in ageing and age associated diseases, including neurodegeneration.

Since oxidatively modified proteins are known to be more susceptible to proteolytic degradation by the proteasome than native proteins, it seems obvious that the proteolytic activity of the proteasome is required more and more with increasing age. In contrast, it was known that the proteasome activity is progressively decreased with increasing age particularly by accumulating oxidized and cross-linked cellular proteins.

Therefore, the age-related accumulation of oxidized proteins may result from either an increase in protein oxidation or a decline in the degradation of oxidized proteins.

Therefore, timely removal of oxidatively damaged proteins is of critical importance to maintain normal cellular homeostasis and viability. In any case, attempts to improve proteostasis pharmacologically would probably have to occur at an early stage of disease before the manifestation of severe cellular dysfunction (Hipp etal., 2014).

The proteasome is the cell's first defense mechanism against accumulating proteotoxic stresses induced by oxidative damage.

The present invention is based on finding that NMDA (N-methyl-D-aspartate) receptor complex antagonists increase the proteasome activity (FIGS. 3A-C;6A-C). The present invention further provides that NMDA receptor antagonists activate the effects of proteasome 20S subunits including chemotrypsin, trypsin and caspase-like activities (FIGS. 6, 7 and 8). According to some embodiments of the invention, the NMDA receptor antagonists mainly activate the proteasome 20S subunit independently of UPS (Ubiquitin-ATP-Dependent Proteolysis). Results from the series of experiments support this conclusion (FIGS. 12 to 22).

Therefore, without wishing to be bound by any theory or mechanism of action, the present invention claims that the NMDA receptor antagonist increase proteasome 20S activities including chemotrypsin, trypsin and caspase-like activities.

The decline of the total rates of protein degradation contributes to a catastrophic imbalance in proteostasis and accumulation of oxidatively damaged and/or misfolded proteins. Therefore, the present invention provides compositions and methods for treatment of the diseases caused by the oxidatively damaged and/or protein-misfolding disorders by increasing the proteasome activity.

The present invention encompasses the finding that activation of proteasome by NMDAR blockers, through increased levels and/or increased activity, can provide effective treatment for, and even prophylaxis of, certain proteinopathies.

The present invention specifically encompasses the insight that, in some instances, increased chemotrypsin, trypsin and caspase-like activities of proteasome can provide effective treatment (and/or prophylaxis) of certain proteinopathies.

Thus, the present invention provides, in one aspect, a pharmaceutical composition comprising at least one NMDA receptor antagonist or a pharmaceutically acceptable salt, hydrate or pharmaceutically active enantiomer thereof, for use in treating the oxidatively damaged and/or protein-misfolding diseases. The disorders', related to the amyloidogenic intrinsically disordered proteins may comprise, but are not limited to amyloid β peptides, tau (Alzheimer's disease), α-synuclein (Parkinson's disease and other synucleinopathies), TAR DNA-binding protein 43, fused in sarcoma (FUS) protein (amyotrophic lateral sclerosis (ALS)), huntingtin (Huntington's disease), a number of human functional amyloids (cytoplasmic polyadenylation element-binding protein (CPEB), T-cell-restricted intracellular antigen-1 (TIA-1), premelanosome protein 17 (Pmel17), secretory peptide hormones), and cell cycle related proteins including p53, p27, p21 (cell cycle, apoptosis) (FIG. 1A) and others (FIGS. 4A and B, 29A-C and 30).

In some embodiments, the present invention provides a proteasome activation by NMDAR blockers as a new therapeutic approach to target proteotoxic disorders. In some embodiments, this invention provides that the new treatment protocol is related to the activation of proteasome 20S as a target.

Therefore, the present invention provides activation of 20S proteasome which exists mainly as the latent in aged individuals, who are the major victim of proteotoxic disorders and diseases. Therefore, the present invention targets 20S proteasome, which is one of the major target in preventing of aging which is associated proteotoxic disorders which is often associated with multiple human diseases. Furthermore, the present invention provides that targeting 20S protesome is effective in treating and preventing of aging. This may be in part because of the fact that the 20S proteasome is more resistant to oxidative stress than the 26S proteasome as the 20S complex can maintain activity even upon treatment with moderate to high concentrations of H₂O₂, whereas the 26S proteasome is much more vulnerable (FIGS. 23A and B).

According to an aspect of some embodiments of the present invention there is provided an NMDA receptor antagonist for use in treating aging and/or aging related conditions or diseases dependent protesome trypsin, chymotrypsin, and caspase-like activities decrease.

Proteins are one of the major targets of oxygen free radicals and other reactive species. A constant accumulation of oxidized proteins takes place during aging.

Oxidative modification of proteins by oxygen free radicals and other reactive species such as hydroxynonenal occurs in physiologic and pathologic processes. As a consequence of the modification, carbonyl groups are introduced into protein side chains by a site-specific mechanism (Jung et al 2014).

The present invention further provides compositions and methods that prevents, reduces or even treats oxidatively damaged protein in the tissues by showing a reduction in carbonylation of proteins (FIG. 28).

Therefore, the present invention provides compositions and methods not only treat diseases, but more importantly, prevent the causes of diseases which is associated with malfunctioning of the protein homeostasis network including accumulation of oxidatively damaged and/or misfolded proteins.

In another embodiment, the present invention demonstrated that NMDA receptor inhibitors both ketamin and memantin prevented cell senesence as a result of the experiment with β-galactosidase staining and cell proliferation assay of primary fibroblast cells (FIG. 24A,B).

The present invention also demonstrated that memantine increased life span of C. elegans worms. The present invention demonstrated that at the day 21, 4 times more worms survived in the group that received the memantin compared to group with the no-treatment (FIG. 25A). The present invention further demonstrate that the worm's locomotor activities increase dramatically in the presence of memantine (FIG. 25C).

NAD+/NADH and NADP+/NADPH are involved in various biological processes in mammalian cells. Although NAD+ and NADP+ are synthesised in sufficient amounts under normal conditions, shortage in their supply due to over consumption and their decreased synthesis has been observed with increasing age and under certain disease conditions. Several studies have proved that in a wide range of tissues, such as liver, skin, muscle, pancreas, and fat, the level of NAD+, NADP+ decreases with age. Therefore the amount of NADP+, NADPH+ and their ratio were investigated in C. elagans treated with memantine. It was found that 2 times more NADP+ detected in the C. elagans treated with memantine compared to the C. elegans without memantine on the 19th day (FIGS. 25F and G).

Therefore, present invention provide a mediciment increase both the healthspan and lifespan.

Many biomarkers related to aging have been identified in human. These biomarkers have been similarly identified in mice. Mice are used to model of human aging and many motor disorders, both somatic and central nervous system in orgin. For all of these models, a full assessment of their motor deficients must include specific test of strength.

In some embodiments, the present invention demonstrated a statistically significant dramatic increase in clinical fraility tests performed including, Kondziela's inverted screen tests (Latency of falling time, mobility time, four fingers grip), weight test (forelimb grip-holding time), and Open Field Maze (OFM) tests (total distance travel, total Rearing number tests, Supporting Rearing Number, Unsupporting Rearing Number) in groups of 2-year-old mice receiving memantine compared to not receiving group of mice (FIG. 27A-K).

The simplest and most easly determined findings of clinical signs of deterioration in aging mice are the evaluation of changes in mouse skin. Indeed, the color change and condition of mouse fur in 22 months of age with and without memantine was very different in favor of those who received memantine (FIG. 26).

The present invention encompasses the finding that activation of proteasome by NMDAR blockers, through increased levels and/or increased activity, can provide effective treatment for, and even prophylaxis of, age related emotional anxiety in addition to providing improvement in locomotor activities.

In above studies, we have shown that memantine increase the life span of C. elegans, as well as increase their muscle strength. In mouse studies, we have shown that memantine phenotypically increases muscle strength, decreases amyloid formation (FIG. 29A), decreases melanin and lipofuscin deposition (FIGS. 30A and B), fat deposition (FIG. 31A-D), and decreases oxidation of proteins in tissues, which are markers of the aging, dramatically, we did not investigate the direct effect of memantine on mouse lifespan.

The most important study supporting our invention is the study which published and patented by Anthony FUTERMAN, Andres David KLEIN showing that memantine increase life span of 4 different strains of 15 mice with GD symptoms

Futerman et al. showed that by using NMDAR blockers including, memantine in all mice with Gaucher disease (GD) symptoms, their lifespan extended and their motor coordination improved significantly (FIGS. 32A-D).

It is known that multiple proteinopathies are pathophysiologic features of GD.

Therefore, in fact, Futerman et al. disrupted protein homeostasis network by inhibiting protein degredation capacity by inhibiting GCase actually provided premature aging associated with interferes with crucial signaling pathways including autophagy-lysosome and proteasome.

Further, the present invention provides compositions and methods effective in ameliorating onset and/or progression of the ageing. In particular the compositions of the present invention diminish or prevent symptoms of the ageing through increasing the proteasome activity.

Therefore, the present invention provides compositions and methods for treatment of aging which is associated with malfunctioning of the protein homeostasis network and interferes with crucial signaling pathways and is often associated with multiple human diseases.

According to an aspect of some embodiments of the present invention, there is provided an NMDA receptor antagonist for use in detoxifying the cells with oxidatively damaged proteins by inducing the activity of the 20S proteasome complex which is capable of directly targeting oxidatively damaged proteins to detoxify the cell (FIGS. 23A and B).

Therefore, the present invention provides compositions and methods not only treat diseases, but more importantly, treat the causes of diseases which is associated with malfunctioning of the protein homeostasis network including accumulation of oxidatively damaged and/or misfolded proteins.

According to an aspect of some embodiments of the present invention provides compositions and methods effective in ameliorating onset and/or progression of the ageing which is associated with malfunctioning of the protein homeostasis network including accumulation of oxidatively damaged and/or misfolded proteins.

Therefore, the present invention provides compositions and methods which prevents the malfunctioning of the protein homeostasis network including accumulation of oxidatively damaged and/or misfolded proteins, which are shown as the most important causes of aging, and provides prolongation of life.

Many literature exists that on increased amounts of oxidized proteins in skeletal muscle, heart, skin, liver, lymphocytes and other tissues of various animal species during aging. Therefore, the present invention provides compositions and methods which maintain and increase muscle mass, strength and performance. In addition, the irreversible loss of cardiomyocytes due to oxidative stress is the main cause of heart dysfunction following ischemia/eperfusion injury and ageing-induced cardiomyopathy. Therefore, the present invention provides compositions and methods which prevents heart diseases by maintaining and/or improving the performance of the heart muscle, and is effective in ameliorating onset and/or progression of the heart dysfunction following ischemia/reperfusion injury and ageing-induced cardiomyopathy.

In another embodiment the present invention provides a pharmaceutical composition comprising at least one NMDA receptor antagonist or a pharmaceutically acceptable salt, hydrate or pharmaceutically active enantiomer thereof, for use in preventing the metabolic syndrome by regulating protein homeostasis and protein quality control

The present invention encompasses the finding that activation of proteasome by NMDAR inhibitors, through adjusting the malfunctioning of the protein homeostasis network including accumulation of oxidatively damaged and/or misfolded proteins, can provide effective treatment for, and even prophylaxis of, the obesity and obesity associated disorders including cardiovascular diseases, insulin resistance, diabetes mellitus, dyslipidemia.

The age-related reduction in fat oxidation, therefore, may promote the accumulation of total and central body fat. Adipogenesis and lipid accumulation during aging have a great impact on the aging process and the pathogenesis of chronic, age-related diseases. The aging associated with malfunctioning of the lipid-protein homeostasis network and interferes with crucial signaling pathways and is often associated with multiple human diseases including PD.

Therefore, the present invention provides compositions and methods which prevents adipogenesis and lipid accumulation during aging by maintaining and/or improving the performance of the lipid-protein homeostasis network.

In addition, the present invention provides compositions and methods ameliorating onset and/or progression of the adipogenesis and lipid accumulation by maintaining and increasing the muscle mass, strength and performance since a reduction in the size and/or oxidative capacity of the metabolically-active muscle mass is an important determinant of reduced fat oxidation. Decreased muscle mass and function are often closely related to aging and metabolic syndromes including (i) mitochondrial dysfunction, which causes various skeletal muscle (SkM) pathologies such as sarcopenia and muscular dystrophy, (ii): inflammatory myopathy caused by inflammation and oxidative stress, such as dermatomyositis, polymyositis, necrotizing autoimmune myositis, and sporadic inclusion body myositis, and (iii) chronic diseases that cause SkM damage, such as type 2 diabetes, obesity, chronic kidney disease, and chronic obstructive pulmonary disease. These syndromes often lead to a decline in the quality of life of patients over time and increase patient mortality.

Therefore, the present invention further provides compositions and methods which prevents the aging and metabolic syndromes by increasing the muscle mass and activity.

Therefore, in another embodiment the present invention provides a pharmaceutical composition comprising at least one NMDA receptor antagonist or a pharmaceutically acceptable salt, hydrate or pharmaceutically active enantiomer thereof, for use in preventing the metabolic syndrome by regulating protein homeostasis and protein quality control.

As indicated above present invention demonstrated that locomotor activities of both C. elegans and mice treated with NMDAR blockers was significantly increased than in the non-NMDAR inhibitor-treated controls. In addition, present invention demonstrated that amount of lipid droplets in muscle tissues of both C. elegans and mice treated with NMDAR inhibitor was significantly less than in the non-NMDAR inhibitor-treated controls.

Thus, the present invention provides, in one aspect, a pharmaceutical composition comprising at least one NMDA receptor antagonist or a pharmaceutically acceptable salt, hydrate or pharmaceutically active enantiomer thereof, for use in preventing the metabolic syndrome by increasing muscle mass and activity and accordingly regulates lipid metabolism.

In addition, the present invention provides, in one aspect, a pharmaceutical composition comprising at least one NMDA receptor antagonist or a pharmaceutically acceptable salt, hydrate or pharmaceutically active enantiomer thereof, for use in preventing the obesity and related diseases which occur in conjunction with metabolic syndrome caused by the changes in lipid metabolism with age.

Therefore the present invention encompasses the finding that activation of proteasome by NMDAR blockers, through increased levels and/or increased activity, is useful in the prevention, amelioration and/or treatment of disorders of the metabolism influencing body weight, in particular in the treatment of obese subjects, in particular in the treatment of human Subjects.

In accordance with this invention it is also envisaged that NMDA receptor antagonists, in particular of memantine, a pharmaceutically acceptable salt or a prodrug of NMDA receptor antagonists is employed in the medical intervention of secondary disorders related to a (pathological) increase of body weight. These ‘secondary disorders’ may comprise, but are not limited to diabetes type 2, high blood pressure (hypertension), cardiovascular diseases, cancer, problems with sexual function and disorder of the muscular or bone system.

The present invention provides compositions and methods which maintain and/or decrease the aging of skin and hair by decreasing accumulation of oxidized protein and lipids and inflammation. Therefore, present invention according to an aspect of some embodiments of the present invention there is provided an NMDA receptor antagonist for use in detoxifying the cells with oxidatively damaged proteins and lipids by inducing the activity of the 20S proteasome complex which is usable in cosmetics as anti-ageing.

The present invention provides compositions and methods which maintain and increase the sternness characteristics which is the potential for self-renewal and for multilineage differentiation and lineage reprogramming of stem cells. In addition, present invention demonstrates that the usage of NMDAR blockers increase the lifespan of human mesenchymal stem cells (FIGS. 36A-D).

With an increase in age, the physiological activity of muscle stem cells decreases and the regeneration capacity of SkM declines and the efficiency of muscle repair decreases, and this is accompanied by a decrease quality and strength of muscles, and irreversible muscle loss which seriously affect the quality of life of the elderly. Therefore, the present invention provides compositions and methods which maintain and increase the stemness characteristics and prevent a decrease quality and strength of muscles, irreversible muscle loss, and muscle diseases which seriously affect the quality of life of the elderly by increasing proteasome activity.

The effects of the present invention is also not limited to muscle tissue, but also the invention provides compositions and methods which maintain and increase the stemness characteristics and prevent aging and associated diseases by ensuring the continuation of the regenerative character of all tissues including neurons.

The present invention provides compositions and methods which prevent and/or treat ocular pathologies of high incidence, such as age-related macular degeneration, cataracts, glaucoma, and diabetic retinopathy which are of multifactorial origin and are associated with genetic, environmental factors, age, and oxidative stress, among others; the latter factor is one of the most influential in ocular diseases, directly affecting the processes of autophagy activity. Depending on the context, autophagy and the proteasome share common substrates as well as regulatory factors. Both systems intersect and communicate at multiple points to coordinate and balance their actions in proteostasis and homeostasis of organelles.

The most important concern that may occur as consequence of decreased protesome activity is the accumulation of potentially toxic un/misfolded proteins as well as protein aggregates. The proteasome impairment will eventually affect the vital function of cytosolic processes including autophagy and all other organelles due to the accumulation of unfolded and damaged proteins.

According to an aspect of some embodiments of the present invention there is provided an NMDA receptor antagonist for use in treating or preventing ocular pathologies related with impaired protein organel turnover by increasing the proteasome activity and, accordingly, it acts by regulating autophagy-lysosome pathway.

According to an aspect of some embodiments of the present invention there is provided an NMDA receptor antagonist for use in treating viral enfections by degredading proteins of viruses, such as HepC core protein. Another subject of this invention is HIV because proteasome inhibitors are known to activate the latent HIV (Cary and Peterlin 2020; Timilsina etal. 2019).

As mentioned above present invention showed that NMDAR inhibitors decrease in carbonylated and carbomylated proteins which am responsible for the structural changes lead to the exposure of abnormally destructured areas on the protein surface generating neoantigens, which are responsible for autoimmune reactions.

Therefore, in some embodiments, the present invention provides compositions and methods which prevents the autoimmunity against new antigenic structures that occur as a results of structural changes in proteins due to malfunctioning of the protein homeostasis network including accumulation of oxidatively damaged, carbonylated, carbomylated and/or misfolded proteins.

In another embodiments the present invention provides chemical structures and/or methods that will maintain and/or increase proteosis and thus ameliorate onset and/or progression and even treatment of the autoimmune responses arised against protein aggregations caused by oxidatively damaged and/or misfolded proteins.

The present invention provides an enhancement of proteolytic capacity by increasing proteasome activity by using of at least one NMDA receptor antagonist, therefore, provides chemical structures and/or methods that will maintain and/or increase proteosis and thus ameliorate onset and/or progression and even treatment of the infectious diseases and autoimmune diseases.

The present invention provides methods and compositions related to diverse group of neurodegenerative proteinopathies that are linked to intracellular and/or extracellular accumulation of specific protein aggregates, such as Tau (FIG. 4A). In many cases, it is thought that the protein aggregates exert toxic effects on the brain, and contribute to disease pathology.

In another aspect, present invention demonstrates that increased protesome activity in the presence of NMDAR blockers decrease serin racemesa, which decrease L-to-D-serin conversion (FIG. 4B), which will be effective in preventing or treating diseases of cerebral ischemia, traumatic brain injury, peripheral neuropathy, cerebral ischemia, choroidal neovascularization, Hyperactivity brain syndrome, retinal ganglion cell loss, Long-term Sequelaes of concussion, Seizure, retinal ganglion cell loss in diabetics, chronic social defeat stress, and other neurodegeneratif diseases.

Furthermore, the present invention provides compositions and methods which reduce the toxic effects of astrocytes by decreasing serin racemesa. Therefore, present inventions further provide a decrease in microvascular damage in diabetic retinopathy, synaptic damage after traumatic brain injury, neuronal over-activation, choroidal neovascularization, the pathologic symptoms of Alzheimer's disease, and neurodegeneration.

In another aspect, present invention provides compositions and methods which act as neuro-protective in neurological diseases.

The present invention provides compositions and methods which prevent and/or treat neurodegeneration caused by pathological insults including anticancer treatments, cerebral ischemia, traumatic brain injury, peripheral neuropathy, cerebral ischemia, choroidal neovascularization, retinal ganglion cell loss, Long-term Sequelaes of concussion, decreasing neurotoxic effects of astrocytes in Alzheimer's disease, Seizure (Saez-Atienzar and Masliah, 2020; Glass et all. 2010; Pender etall. 2020)

In another aspect, present invention demonstrates that increased protesome activity in the presence of NMDAR blocker decrease serin racemesa, which decrease L-to-D-serin conversion, which will be effective in preventing or treating schizophrenia and other psychological diseases.

Tauopathies are neurodegenerative disorders characterized by the presence of filamentous deposits, consisting of hyperphosphorylated tau protein, in neurons and glia. Abnormal tau phosphorylation and deposition in neurons and glial cells is one of the major features in tauopathies (Weng and He 2021)

In addition It has been reported that tau protein abnormal hyperphosphorylation plays a central role in neurodegeneration triggered by traumatic brain injury (TBI) and Traumatic spinal cord injury. Exemplary such tauopathies include amytrophic lateral sclerosis (ALS), parkinsonism, argyrophilic grain dementia, diffuse neurofibrillary tangles with calcification, frontotemporal dementia linked to chromosome 17, corticobasal degeneration, Pick's disease, progressive supranuclear palsy, progressive subcortical gliosis, and tangle only dementia.

Ever-mounting evidence suggests that the ubiquitin proteasome system (UPS) deficits contribute to p-tau accumulation. And targeting UPS attenuates tau pathology.

The present invention provides methods relevant to tauopathies. In some embodiments, the present invention provides a method of demonstrates that increased protesome activity in the presence of NMDAR blocker reduce the accumulation of pTau proteins in a cell (FIG. 4B), the method comprising administering to a cell a therapeutically effective amount of such a provided NMDAR blockers. In some embodiments, the cell is a neuronal cell. In some embodiments, the cell is a non-neuronal cell. In some embodiments, the cell expresses tau proteins. In certain embodiments, the tauopathy is Alzheimer's disease.

Therefore, in one aspect, present invention provides compositions and methods which prevent and/or treat diseases mentioned above caused by the tauopathy.

The present invention provides methods that prevents, reduces or even treats the cognitive impairment and dementia which may stem from any etiology.

Previously described by Tedeschi and Dupraz (Tedeschi and Dupraz 2019) that the actin turnover provide an increase in axon regeneration which prevent and/or treat neurodegeneration caused by pathological insults. The present invention provides, in another aspect, a finding that NMDAR blocker causes actin turnover by increasing the proteasome activity (FIG. 35) and, therefore, may provide an increase in axon regeneration. Therefore, the present invention provides compositions and methods which prevent and/or treat neurodegeneration caused by pathological insults including anticancer treatments, cerebral ischemia, traumatic brain injury, peripheral neuropathy, cerebral ischemia, choroidal neovascularization, retinal ganglion cell loss, long-term sequelaes of concussion, decreasing neurotoxic effects of astrocytes in Alzheimer's disease. Therefore, neurodegenerative diseases are selected from the group consisting of neurodegeneration caused by pathological insults including anticancer treatments, cerebral ischemia, traumatic brain injury, peripheral neuropathy, cerebral ischemia, choroidal neovascularization, retinal ganglion cell loss, long-term Sequelaes of concussion, decreasing Neurotoxic effects of astrocytes in Alzheimer's disease, seizure.

In one aspect present invention provides compositions and methods which prevent and/or treat renal diseases caused by accumulation of oxidatively damaged and/or misfolded proteins (Cauli et al. 2014).

In another aspect present invention provides compositions and methods which prevent and/or treat renal insufficiencies caused by aging, hyperlipidemia, hypertension, smoking, diabetes, obesity and other factors which cause oxidative damage to proteins. Therefore, present invention ameliorate and/or treat acute and chronic kidney disease.

In another aspect present invention provides a method for treating colorectal cancer by decreasing Serine racemase which enhances growth of colorectal cancer by producing pyruvate from serine (Ohshima et al. 2020).

The present invention provides methods that prevents, reduces or even treats the cell proliferative diseases by reducing or preventing the unfolded protein accumulation.

Therefore, the present invention further provides, in another aspect, a method for treating different forms of cancer, in which protein homeostasis network is disturbed including increased protein synthesis, and misfolded protein production, accumulation of both misfolded protein aggregates, as well as apoptosis signaling proteins in cancer cells. In addition the present invention further provides methods that increase the sensitivity of cancer cells to antineoplastic drugs (Madden et al. 2019; Hsu et al. 2019).

The shortening of telomere length is one of metabolic derangements compromise the normal aging process (such as loss of muscle mass and strength). Previous studies have shown that inhibition of proteasome activity inhibits telomerase activity (Shalem-Cohavi et al. 2019).

Therefore, in another aspect, the present invention provides that NMDA receptor antagonists increase telomerase activity by increasing the activity of proteasome. Consequently, in another aspect, present invention provides a method that NMDA receptor antagonists delays cell senescence andi accordingly, delay in aging process by increasing telomerase activity which provides prolongation of life.

The present invention encompasses the finding that activation of proteasome by NMDA inhibitors, through increased levels and/or increased activity, can provide effective treatment for, and even prophylaxis of the endoplasmic reticulum (ER) related diseases by decreasing ER stress. Metabolic diseases including, Insulin resistance, arteriosclerosis, diabetes mellitus, obezite, alcoholic and non-alcoholic fatty liver disease, hyperlipidemia; cancers including Leukemia, multiple myeloma, breast cancer, prostate tumor, immune system related diseases including, viral infections, Bacterial infections, Vitiligo, rheumatoid arthritis, type 1 diabetes and many neurological diseases are known to be associated with ER stress (Ding et al. 2007; Roussel et al. 2013).

The present invention encompasses the finding that activation of proteasome by NMDA inhibitors, through increased levels and/or increased activity, can provide effective treatment for, and even prophylaxis of, certain Lysosomal Storage Diseases. The present invention encompasses the finding that activation of proteasome by NMDA inhibitors, prevents the malfunctioning of the protein homeostasis network including accumulation of oxidatively damaged and/or misfolded proteins which are associated with lysosome dysfunctions. Lysosomal storage diseases include Gaucher disease, Fabry disease, Niemann-Pick disease, Hunter syndrome, Glycogen storage disease II (Pompe disease), Tay-Sachs disease.

The present invention can provide effective treatment for, reduction of symptoms and even prophylaxis of, certain mitochondrial diseases including. Mitochondrial myopathy, Diabetes mellitus, Leber's hereditary optic neuropathy. Leigh syndrome, subacute sclerosing encephalopathy. Neuropathy, ataxia, deafness, retinitis pigmentosa. and ptosis, progressive symptoms of dementia, Myoneurogenic gastrointestinal encephalopathy, MERRF syndrome, MELAS syndrome, Huntington's disease, cancer, Alzheimer's disease, Parkinson's disease, bipolar disorder, schizophrenia, aging and senescence, anxiety disorders.

Autophagy lysosome pathway is another degradation pathway in which cytoplasmic components, such as protein aggregates and damaged organelles are degraded and recycled for maintaining normal cellular homeostasis. Depending on the context, autophagy and the proteasome share common substrates as well as regulatory factors. Both systems intersect and communicate at multiple points to coordinate and balance their actions in proteostasis and homeostasis of organelles (Kocaturk and Gozuacik, 2018).

The present invention can provide effective treatment for, reduction of symptoms and even prophylaxis of, certain autophagy related human diseases, such as Adult neurodegenerative Disorders including Parkinson's disease, Amyotrophic lateral schlerosis, Frontotemporal dementia, Neuronal ceroid lipofuscinosis, Fulminant neurodegeneration, Dementia with Lewy bodies; Pediatric Neurodevelopmental disorders including Spinocerebellar ataxia, Cortical atrophy and epilepsy, Childhood-onset neurodegeneration, BPAN, Spastic quadriplegia and brain abnormalities, Primary microcephaly, Hereditary spastic paraplegia, Ataxia with spasticity, Rett syndrome, Joubert syndrome, Leukoencephalopathy, Adolescent-onset dystonia, CEDNIK syndrome, Pelizaeus-Merzbacher-like disorder, West syndrome; Hereditary neuropathies including Sensory and autonomic neuropathy type II, Charcot-Marie-Tooth disease, Sensory and autonomic neuropathy type IF, Distal hereditary motor neuronopathy; Ophthalmological diseases including Primary open-angle glaucoma, Cataracts; Cardiac and skeletal myopathies including Danon's cardiomyopathy, Distal myopathy with rimmed vacuole, Dilated cardiomyopathy, Sporadic inclusion body myositis, X-linked myopathy with excessive autophagy; Inflammatory disorders including Crohn's disease, Ulcerative colitis, Childhood asthma; Autoimmune diseases including Systemic lupus erythematous, Diabetes, Other autoimmune diseases; Infectious diseases including M. tuberculosis, M. leprae; Skeletal disorders including Osteopetrosis, Paget's disease of the bone, Kashin-Beck disease; Congenital multisystem disorders including Global developmental abnormalities, Vici's syndrome, Zellweger's syndrome, Glycosylation disorder with autophagy defects, Zimmerman-Laband syndrome, Hermansky-Pudlak syndrome, Multisystem proteinopathy.

Targeting cellular senescence

Ablation of senescent cells has been postulated as a promising therapeutic approach to target the ageing phenotype and, thus, to prevent, delay or mitigate ageing-related diseases. The aim with senolytic compounds is to rejuvenate organisms by selectively killing senescent cells and their efficacy is based on the ability of senescent cells to resist apoptosis. In another aspect the present invention demonstrate that NMDA receptor antagonists proteasome activity and cause a decrease in p21, p53, pAkt1 and ppRb which are the major target molecules of the senolytic drugs (FIGS. 1, 2A and 34)

Therefore, the present invention provides, in one aspect, a pharmaceutical composition comprising at least one NMDA receptor antagonist or a pharmaceutically acceptable salt, hydrate or pharmaceutically active enantiomer thereof, for use in ablation of senescent cells which has been postulated as a promising therapeutic approach to target the ageing phenotype and, thus, to prevent, delay or mitigate ageing-related diseases. Therefore, According to an aspect of some embodiments of the present invention there is provided an NMDA receptor antagonist for use in treating ageing which is associated with increasing risk for developing multiple chronic diseases, the geriatric syndromes, impaired physical resilience and mortality (Saez-Atienzar and Masliah 2020.)

Amongst the diseases with emerging evidence for a causal contribution of cellular senescence or benefits of senolytics include, but are not limited to, Diabetes/Obesity, metabolic diseases, Cardiac dysfunction, congestive heart failure, myocardial infarction, Vascular hyporeactivity/calcification, AV fistulae, Frailty, Age-related muscle loss (Sarcopenia), arthritis, osteoporosis, falls, Chemotherapy complications, Radiation complications, Cancers, Bone marrow transplant complications, Organ transplantation complications, Myeloma/MGUS (monoclonal gammopathy of undetermined significance), Age-related cognitive dysfunction, other dementias, Alzheimer's disease, Parkinson's disease, Amyotrophic lateral sclerosis, Ataxia, Obesity-related neuropsychiatric dysfunction, Renal dysfunction, Urinary incontinence, Osteoporosis, Osteoarthritis, Age-related intervertebral disc disease, Idiopathic pulmonary fibrosis, Hyperoxic lung damage, Chronic obstructive pulmonary disease, Tobacco, Hepatic steatosis, Cirrhosis, Primary biliary cirrhosis, Progerias, Pre-eclampsia, Macular degeneration, Glaucoma, Cataracts, blindness, Prostatic hypertrophy, incontinence, Psoriasis, Healthspan, Lifespan and many others.

Importantly, in any case, attempts to improve proteostasis pharmacologically have to be at an early stage of disease before the manifestation of severe cellular dysfunction. Indeed present invention demonstrated that starting treatment early is more effective in increasing life expectancy than starting late (FIG. 25B).

### NMDA Receptor Blockers

In certain embodiments, the NMDA receptor antagonist is selected from the group below consisting of NMDA receptor antagonists are compounds that antagonize, or inhibit, the action of the NMDA receptor. An NMDA receptor antagonist may be a competitive antagonist, an uncompetitive antagonist, a noncompetitive antagonist, and/or a glycine antagonist (Liu et al. 2020; Vieira et al. 2020).

According to some embodiments of the invention, the NMDA receptor is an extra-synaptic NMDA receptor.

The class of uncompetitive ion channel blockers are ketamine, dizocilpine (MK-801), and memantine (Siala et al. 2020; Das 2020).

According to some embodiments of the invention, the NMDA receptor comprises a NR2B subunit.

In certain embodiments, the NMDA receptor antagonist preferentially binds to extra-synaptic NMDA receptors. In certain embodiments, the extra-synaptic NMDA receptors comprise a NR2B subunit.

Non-limiting examples of NMDA receptor antagonists is selected from the group consisting of memantine, nitromemantine, neramexane, ketamine, amantadine, dextromethorphan, L-687,384, amitriptyline, 1-benzyl-6methoxy-6,7′-dihydrospiro [piperidine-4,4′-thieno [3.2-c]pyran], ifenprodil, orphenadrine, kynurenic acid, felbamate, D(−)-AP-5, (+)-CPP, EAA-090, TCN-201, AP-5, AZD6765, SDZ 220-581, (+)-norketamine, eliprodil, dextrorphan, 5,7 dichlorokynurenic acid monohydrate, [Glu3,4,7,10,14]-Conantokin G, D-AP-7, MD-Ada, AP-7, Ro 8-4304, spermine, Ro25-6981, DCOX, traxoprodil, MDL105,519, fanapanel, metaphit, Ro25-6981, NAAG, 5-fluoroindole-2-carboxylic acid, (S)-(−)-4-oxo-2-azetidinecarboxylic acid, benzyl (S)(−)-4-oxo-2-azetidinecarboxylate and (+)-a-amino-3-carbomethoxy-5 methylisoxazole-4-propanoic acid, and pharmaceutically acceptable salts, hydrates and pharmaceutically active enantiomers thereof (Ahmed et al. 2020). Each possibility represents a separate embodiment of the invention.

In certain embodiments, the NMDA receptor antagonist is selected from the group consisting of memantine, nitromemantine, neramexane, ketamine, amantadine, dextromethorphan, L-687,384, amitriptyline, 1-benzyl-6 methoxy-6′,7′-dihydrospiro-[piperidine-4,4-thieno[3.2-c] pyran), eliprodil, ifenprodil, orphenadrine, kynurenic acid, felbamate, and pharmaceutically acceptable salts, hydrates and pharmaceutically active enantiomers thereof. Each possibility represents a separate embodiment of the invention.

In certain embodiments, the NMDA receptor antagonist is selected from the group consisting of memantine, nitromemantine, neramexane, ketamine and pharmaceutically acceptable salts, hydrates and pharmaceutically active enantiomers thereof. Each possibility represents a separate embodiment of the invention.

According to some embodiments of the invention, the NMDA receptor antagonist is selected from the group consisting of memantine, eliprodil and ifenprodil or a pharmaceutically acceptable salt, hydrate or pharmaceutically active enantiomer thereof.

In certain embodiments, the NMDA receptor antagonist is memantine (3,5-dimethyl-1-adamantanamine hydrochloride) or a pharmaceutically acceptable salt, hydrate or pharmaceutically active enantiomer thereof.

According to some embodiments of the invention, the NMDA receptor antagonist is formulated for oral delivery.

In certain embodiments the composition for oral delivery is formulated for sustained release.

In another embodiments of the invention, since the effect of NMDAR blockers (memantin and ketamin) on proteasome activity lasts for 4-8 hours, it should be clinically evaluated whether the NMDAR blocker is givin at 6-8 hours intervals or in the form of a sustained release.

According to some embodiments, the NMDA antagonist is a specific inhibitor of glutamatergic neurotransmission. According to some embodiments, the NMDA antagonist may be a less specific antagonist that acts on other neurotransmission pathways. According to some embodiments, the NMDA antagonist may have additional activities such as serotonergic, cholinergic or dopaminergic antagonist or agonist activities (Paoletti et al. 2013; Petit-Pedrol and Groc, 2021).

In certain embodiments, the NMDA receptor antagonist having nACHR antagonist activity is selected from the group consisting of amantadine and dextromethorphan, and pharmaceutically acceptable salts, hydrates and pharmaceutically active enantiomers thereof. Each possibility represents a separate embodiment of the invention.

In certain embodiments, the NMDA receptor antagonist also has dopaminergic activity. In certain embodiments, the NMDA receptor antagonist is also a dopamine D2 receptor agonist.

In certain embodiments, the NMDA receptor antagonist is also a sigma-1 receptor agonist.

In certain embodiments, the NMDA receptor antagonist having sigma-1 receptor agonist activity is selected from the group consisting of L-687, 384, amitriptyline and 1-benzyl-6-methoxy-6′,7′-dihydrospiro[piperidine-4,4′-thieno[3.2-c]pyran), and pharmaceutically acceptable salts, hydrates and pharmaceutically active enantiomers thereof.

Each possibility represents a separate embodiment of the invention.

A subject in need of such treatment or prevention a therapeutically effective amount of lower than 10 mg/kg of an NMDA receptor antagonist, memantine.

In certain embodiments, the effective amount of the present agent is a dose of about 0.01 to about 10 mg per kilogram of body weight of the subject (mg/kg), i.e., from about 0.01 mg/kg to about 10 mg/kg body weight.

In certain embodiments, an NMDA receptor antagonist is administered to a subject prior to stressor. Wherein it is the accumulation of potentially toxic un/misfolded proteins as well as protein aggregates referred to as stressor. In addition, the proteasome impairment also referred to as stressor dependent on the accumulation of potentially toxic un/misfolded proteins as well as protein aggregates or independent. Therefore, in certain embodiments, the present agent/composition is administered to the subject which is a person 25-year-old or older.

In certain embodiments, the present agent/composition is administered to the subject throughout life.

In certain embodiments, the present agent/composition is administered to the subject which is a person younger than 25-year-old.

In general, an NMDAR blocking agent may be or comprise a compound of any chemical class (e.g., a small molecule, metal, nucleic acid, polypeptide, lipid and/or carbohydrate). In some embodiments, an NMDAR blocking agent is or comprises an antibody or antibody mimic. In some embodiments, an NMDAR blocking agent is or comprises a nucleic acid agent (e.g., an antisense oligonucleotide, a siRNA, a shRNA, etc) or mimic thereof. In some embodiments, an NMDAR blocking agent is or comprises a small molecule. In some embodiments, an activating agent is or comprises a naturally-occurring compound (e.g., small molecule). In some embodiments, an NMDAR blocking agent has a chemical structure that is generated and/or modified by the hand of man. In general, an NMDAR blocking agent increases level or activity of one or more target entities present in and/or produced by a cell or organism, in here, the targets are trypsin, chemotrypin and caspase like actvities of ptotesome. In some embodiments, a target entity is or comprises a polypeptide. In some embodiments, a target entity is or comprises a nucleic acid (e.g., a nucleic acid that encodes or regulates [e.g., by altering expression and/or activity of] a polypeptide). In some embodiments, a target entity is or comprises a carbohydrate. In some embodiments, a target entity is or comprises a lipid. In some embodiments, a target entity is or comprises an enzyme. In some embodiments, a target entity is or comprises a polypeptide involved in cellular trafficking.

Preventing and/or inhibiting the biological function of an NMDA receptor can be effected at the protein level but may also be effected at the genomic and/or the transcript level using a variety of molecules which interfere with transcription and/or translation of a receptor.

Therefore, non-limiting examples of antagonists that can be used according to some embodiments of the invention include small molecules, antibodies, inhibitory peptides, enzymes that cleave the polypeptide, aptamers homologous recombination agents, site specific endonucleases and RNA silencing agents.

The NMDA antagonists and/or the agents of some embodiments of the invention can be administered to an organism per se, or in a pharmaceutical composition where it is mixed with suitable carriers or excipients.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

In certain embodiments, the NMDA receptor antagonist and/or the agents described above is formulated for injection. In certain embodiments the NMDA receptor antagonist and/or the agents are formulated for sustained release.

The scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

Protein synthesis and turnover must be carefully balanced and terminally misfolded proteins effectively removed by proteolysis to ensure proteostasis. Clearance of oxidatively damaged and misfolded proteins is mainly performed by the ubiquitin-proteasome system (UPS), and increasing evidence has clearly demonstrated that the 20S core has a major a role in overall protein degradation, independent of ubiquitin and ATP (Raynes et al. 2016; Hipp et al 2014; Jung et al 2914).

The present invention is based on findings that N-methyl-D-aspartate receptor (NMDAR) complex antagonists increase the proteasome activity (FIGS. 6,7 and 8). The present invention further demonstrates that NMDAR antagonists mainly activate the proteasome 20S subunit independently of UPS (Ubiquitin-ATP-Dependent Proteolysis) (FIGS. 12 to 22).

The present invention further provides that NMDAR antagonists activate the effects of proteasome 20S subunits including chemotrypsin, trypsin and caspase-like activities (FIGS. 6, 7). Impairment of proteostasis is now being recognized as a basic mechanism by which chronic protein misfolding and toxic aggregation cause cellular dysfunction, facilitating the manifestation and progression of numerous neurodegenerative, other aggregate-deposition diseases, and aging and associated diseases (Dobson et al 2015; Chiti and Dobson 2017).

Protein aggregates impair the activity of cellular proteolytic systems (proteasomes), resulting in further accumulation of oxidized proteins. In addition, the accumulation of highly crosslinked protein aggregates leads to further oxidant formation, damage to macromolecules, and, finally, to apoptotic cell death.

Protein aggregates are observed in a variety of different types of disorders, diseases, and/or conditions, including cognitive impairment disorders, proliferative diseases, inflammatory diseases, cardiovascular diseases, immunologic diseases, ocular diseases, mitochondrial diseases, neurodegenerative diseases, lysosomal storage diseases, and aging and associated diseases.

The term “protein aggregate” as used herein refers is to any association of two or more protein molecules in a non-native conformation. Aggregates cover a range of structures, from amorphous assemblies to highly ordered fibrils (amyloid) with cross-b structure. The propensity of a specific protein to aggregate is governed primarily by the chemical properties of its amino acid sequence, the conformational stability of its folded state, and its cellular concentration. The extremely high total protein concentration in cells results in excluded-volume effects (macromolecular crowding) and substantially increases the tendency of non-native protein molecules to aggregate compared with dilute solutions.

A range of proteins involved in these processes (e.g., transthyretin, insulin, β-2-microglobulin, superoxide dismutase) are natively folded. However the vast majority of them involved in protein-aggregate disorders are particularly those that are relatively small, are intrinsically disordered protein (IDPs). Disorder is mostly found in intrinsically disordered regions (IDRs) within an otherwise well-structured protein. The term IDP therefore includes proteins that contain IDRs as well as fully disordered proteins. Intrinsically disordered systems can also be generated following proteolysis from larger proteins that are otherwise folded, such as the amyloid-β peptide and the amyloidogenic fragment of gelsolin. Because of their lack of stable 3D structure, IDPs are extremely sensitive to their environment, much more so than ordered proteins. Also, this lack of fixed structure is often associated with extreme binding promiscuity of IDPs. The best known examples of amyloidogenic IDPs include amyloid β (Aβ) peptides, tau (AD), α-synuclein (Parkinson's disease (PD) and other synucleinopathies), TAR DNA-binding protein 43 (TDP-43), fused in sarcoma (FUS) protein (amyotrophic lateral sclerosis (ALS)), and huntingtin (Huntington's disease). A number of human functional amyloids (cytoplasmic polyadenylation element-binding protein (CPEB), T-cell-restricted intracellular antigen-1 (TIA-1), premelanosome protein 17 (Pmel17), secretory peptide hormones), and cell cycle related proteins including p53, p27, p21 (cell cycle, apoptosis). Morever, twenty-thirty percent of cellular proteins are classified as IDPs and as many as 41% of the eukaryotic proteome is predicted to contain IDRs. Table 1 provides a list of several intrinsically disordered or unknown structured peptides or proteins forming intracellular or extracellular amyloid, nonamyloid, and amyloid-like fibrils deposits in human diseases.

As used herein, the term “amyloidopathy” or “amyloidopathic” refers to diseases, disorders, and/or conditions that are associated with or characterized by pathological accumulation of the any disease-linked protein exhibiting amyloid conformation (i.e., 3-pleated sheet), including but not limited to Alzheimer's disease, vascular dementia, and cognitive impairment.

The term “proteostasis”, or “protein homeostasis”, refers to the concentration, conformation, binding interactions, e.g., quaternary structure, and location of proteins making up the proteome. Proteostasis is influenced by the chemistry of protein folding/misfolding and by numerous regulated networks of interacting and competing biological pathways that influence protein synthesis, folding, conformation, binding interactions, trafficking, disaggregation and degradation.

Increasing proteolytic capacity provides an alternative approach to maintaining proteostasis.

Therefore, the present invention encompasses the finding that activation of proteasome by NMDAR blockers, through increased levels and/or increased activity, can provide effective treatment for, and even prophylaxis of, certain proteinopathies.

For example, the present invention provides novel insights into proteasome activity and its effects on protein degredation, and demonstrates that NMDAR pathways linked to proteasome function regulate levels of protein amount in various contexts, specifically including various cell cultures, C. elegans, mammalian organisms (e.g., mouse), and mouse brain. The present invention specifically encompasses that increased chemotrypsin, trypsin and caspase-like activities of proteasome by NMDAR blocker can provide effective treatment (and/or prophylaxis) of certain proteinopathies which cause cellular dysfunction, facilitating the manifestation and progression of numerous neurodegenerative, other aggregate-deposition diseases, even aging and associated diseases. Some embodiments of the present invention are applicable to all proteinopathies.

The present invention provides an enhancement of proteolytic capacity by increasing proteasome activity by using of at least one NMDA receptor antagonist or a pharmaceutically acceptable salt, hydrate or pharmaceutically active enantiomer. The present invention, therefore, provides a medicament for treating the diseases (Table 1) caused by chronic protein misfolding and toxic aggregation.

In addition to the natural formation of misfolded proteins, the oxidative damage is known to be one of the most important factor on the protein misfolding (Dobson et al 2015; Chiti and Dobson 2017).

Oxidative stress can lead to the non-specific post-translational modifications of proteins and contributes to protein aggregation.

Proteins are one of the major targets of oxygen free radicals and other reactive species. A constant accumulation of oxidized proteins takes place during aging.

Upon oxidative damage, proteins unfold and expose hydrophobic regions which makes them prone to aggregation (Reichmann et al. 20181).

Oxidative modification of proteins by oxygen free radicals and other reactive species such as hydroxynonenal occurs in physiologic and pathologic processes. As a consequence of the modification, carbonyl groups are introduced into protein side chains by a site-specific mechanism and cause proteinopathies (Jung et al 2014). Therefore, protein oxidation play major role in the development of various age-related diseases.

The present invention further provides compositions and methods that prevents, reduces or even treats oxidatively damaged protein in the tissues by showing a reduction in carbonylation of proteins (FIG. 28).

Therefore, the present invention provides compositions and methods not only treat diseases, but more importantly, prevent the causes of diseases which is associated with malfunctioning of the protein homeostasis network including accumulation of oxidatively damaged and/or misfolded proteins.

The failure of cells to maintain proteostasis contributes to the toxic effects of protein aggregates in numerous diseases and is also a major driver of the aging process. Beyond counteracting the toxic effects of aggregating disease proteins, enhancing proteostasis capacity also extends lifespan. Conversely, the chronic presence of aggregates can suppress the ability of cells to respond adequately to stress, supporting the view that protein aggregation is a major driver of the aging process. A decline in proteasome activity during aging would explain why aging is a key risk factor for protein aggregation. Conversely, there is evidence that aggregation is not the result of a malfunctioning UPS but is actually its cause. This view is supported by findings that the expression of structurally unrelated aggregation-prone proteins prevents other proteins from being proteasomally degraded (Chiti and Dobson 2017).

Modulation of protein concentration via regulation of the proteolytic machineries has long been validated as promising milieu for the development of treatments for ageing and ageing related different human diseases such as neurodegeneration, cancer, autoimmunity and others.

Therefore, clearance of misfolded proteins is critical for cell survival because misfolding alters a protein's three-dimensional structure, impairing its biological activities and increasing its propensity to form toxic aggregates.

Therefore, it has become important to investigate chemical structures and/or methods that will maintain and/or increase proteosis and thus ameliorate onset and/or progression and even treatment of the ageing and aging associated diseases.

The method of the invention comprises administering to the subject a therapeutically effective amount of an NMDA receptor antagonist effective in increasing the activity of proteasome. The invention is directed to means and methods effective in protecting the onset and/or progression and even treatment of the ageing.

The terms “treating” and “treatment” as used herein refer to abrogating, substantially inhibiting, slowing or reversing the progression of a condition, substantially delaying the appearance of clinical symptoms of a condition, substantially ameliorating clinical symptoms of a condition or substantially preventing the appearance of clinical symptoms. For example, in relation to aging, the term “treat” may mean to relieve or alleviate susceptibility to infection, risk of heat stroke or hypothermia, thinning of the bones of spines, bones breaks, joint changes (ranging from minor stiffness to severe arthritis), loss of muscle mass and strength (sarcopenia), stooping posture, slowed and limited movement, hand strength and mobility decrease include, “hand and finger strength and ability to control submaximal pinch force and maintain a steady precision pinch posture, manual speed, and hand sensation, Frailty, a syndrome of decreased strength, physical activity, physical performance and energy, VO2 max and maximum heart rate decline, constipation, urinary incontinence, slowing of thought, memory, and thinking, reduced reflexes and coordination and difficulty with balance, decrease in visual acuity, diminished peripheral vision, hearing loss, wrinkling and sagging skin, whitening or graying of hair, weight loss, in part due to loss of muscle tissue, losing strength in the ciliary muscle of the eyes which leads to difficulty focusing on close objects, or presbyopia, menopause typically occurs between 44 and 58 years of age, loss of arterial elasticity and as a result causes the stiffness of the vasculature, atherosclerosis which leads to cardiovascular disease (for example stroke and heart attack) which globally is the most common cause of death.

In relation to aging, the term “protect” is used herein to mean prevent delay or treat, or all, as appropriate, development or continuance or aggravation of conditions related to aging of human body systems. Following are examples of how aging affects some of our major body systems;

Cells, organs and tissues: Cells become less able to divide, The telomere shortening, oxidasied products accumulation, connective tissue stiffness between the cells, the decrease in maximum functional capacity of many organs.

Heart and blood vessels: The increase in the thickness wall of the heart gets thicker. Heart muscle become less efficient (working harder to pump the same amount of blood). The aorta (the body's main artery) becomes thicker, stiffer, and less flexible. Many of the body's arteries, including arteries supplying blood to the heart and brain, slowly develop atherosclerosis, although the condition never becomes severe in some people

Vital signs: It is harder for the body to control its temperature. Heart rate takes longer to return to normal after exercise.

Bones, muscles, joints: Bones become thinner and less strong. Joints become stiffer and less flexible. The cartilage and bone in joints starts to weaken.

Muscle tissue becomes less bulky and less strong

Digestive system: The movement of food through the digestive system becomes slower. The stomach, liver, pancreas, and small intestine make smaller amounts of digestive juices

Brain and nervous system: The number of nerve cells in the brain and spinal cord decreases. The number of connections between nerve cells decreases. Abnormal structures, known as amyloids, plaques and tangles, may form in the brain. Cognitive impairment (such as impairment of memory and/or orientation) or impairment of global functioning (overall functioning, including activities of daily living) and/or slow down or reverse the progressive deterioration in global or cognitive impairment.

Eyes and Ears: The retinas get thinner, the irises get stiffer, The lenses become less clear. The walls of the ear canal get thinner. The eardrums get thicker.

Skin, nails, and hair: Skin gets thinner and becomes less elastic. Sweat glands produce less sweat. Nails grow more slowly. Hairs get gray and some no longer grow

According to the present invention, all of the molecular and phenotypic changes that occur after the use of NMDAR blockers, noted in the following results of the studies, were the result of NMDAR blocker's increasing protesome activity, unless otherwise stated. Protesome activation basically comprises activation of 20S proteasome and its functions including trypsin, chemotrypsin and caspase-like activities.

The present invention provides compositions and methods effective in ameliorating onset and/or progression of the ageing by preventing the malfunctioning of the protein homeostasis network including accumulation of oxidatively damaged and/or misfolded proteins.

The contribution of oxidative stress in age-related organ changes seems generally agreed that increases in ROS accompany aging, leading to functional alterations, increased incidence of disease, and a reduction in life span (Hipp et all 2014).

The proteasome is the cell's first defense mechanism against accumulating proteotoxic stresses induced by oxidative damage (Korovila et al. 2017; Shang et al. 1997; Grune, 2000; McNaought et al. 2001, Njoman and Tepe 2019).

Therefore, the present invention provides compositions and methods effective in ameliorating onset, progression, and even prevention of the ageing. In particular the compositions of the present invention diminish or prevent symptoms of the ageing through maintaining proteosis by increasing the proteasome activity. In addition present invention demonstrate that that usage of NMDA receptor complex antagonists not only decrease the ageing and aging associated diseases but also increase life span. In another words, the present invention provides an increase lifespan together with healthspan.

In simple terms, life span is the number of years in life, whereas healthspan is the quality of life. Healthspan covers the amelioration, preventation, and/or correction of the changes in the body systems that occur with age and the clinical findings related to these, as specified in the above paragraphs.

The present invention showed that NMDA receptor inhibitors both ketamin and memantin prevented cell senesence as a result of the experiment with β-galactosidase staining of primary fibroblast cells (FIG. 24A). In addition The present invention also showed that NMDA receptor inhibitors increase the proliferation abilities and fat metabolism of primary fibroblast cells (FIGS. 24C and D).

The present invention also demonstrated that memantine increased life span of C. elegans worms. The present invention demonstrated that at the day 21, 4 times more worms survived in the group that received the memantin compared to group with the no-treatment (FIGS. 25A and B). The present invention further demonstrate that the worm's locomotor activities increase dramatically in the presence of memantine (FIG. 25C).

Therefore, present invention provide a mediciment increase both the healthspan and lifespan.

The present invention further demonstrate that NMDAR blocker increases the amount of NADP+ and increases the ratio of NADP+/NADPH.

Coenzyme I (nicotinamide adenine dinucleotide, NAD+/NADH) and coenzyme II (nicotinamide adenine dinucleotide phosphate, NADP+/NADPH) are involved in various biological processes in mammalian cells. Although NAD+ and NADP+ are synthesised in sufficient amounts under normal conditions, shortage in their supply due to over consumption and their decreased synthesis has been observed with increasing age and under certain disease conditions. Several studies have proved that in a wide range of tissues, such as liver, skin, muscle, pancreas, and fat, the level of NAD+, NADP+ decreases with age. The ratio of NAD+/NADH and NADP+/NADPH indicates the cellular redox state. A decrease in this ratio affects the cellular anaerobic glycolysis and oxidative phosphorylation functions, which reduces the ability of cells to produce ATP (She et al. 2021).

The molecule exists in cells in reduced (NADPH) and oxidized (NADP+) forms reflecting the redox state of the cell. An important cellular role for NADPH is to provide the reducing power for reductive biosynthesis of macromolecules including fatty acids and complex lipids, proteins, and nucleotides. NADPH also plays an important role in the control of cellular redox state. In this latter function, NADPH provides reducing equivalents for reduction of oxidized glutathione (GSSG) to the reduced form (GSH), a reaction catalyzed by glutathione reductase. GSH is an important intracellular reductant and plays an important role in cellular anti-oxidant defense.

Therefore the amount of NADP+, NADPH+ and their ratio were investigated in C. elagans treated with memantine. It was found that 2 times more NADP+detected in the C. elagans treated with memantine compared to the C. elegans without memantine on the 19th day (FIGS. 25F and G).

The age-related reduction in fat oxidation, therefore, may promote the accumulation of total and central body fat. Adipogenesis and lipid accumulation during aging have a great impact on the aging process and the pathogenesis of chronic, age-related diseases. Therefore, in the presence of Memantine and control C. elegans groups on 20th days, the amount of fat in the organism was investigated by Nile Red staining. The results of the invention show that, on the 20th day, lipid accumulation in the control C. elagans group was more than 2-fold higher than the C. elegans grown in the presence of memantine (FIGS. 25D and E).

The simplest and most easly determined findings of clinical signs of deterioration in aging mice are the evaluation of changes in mouse skin. Indeed, the present invention further demonstrate that the color change and condition of mouse fur in 22 months of age with and without memantine was very different in favor of those who received memantine (FIG. 26).

Many biomarkers related to aging have been identified in human. These biomarkers have been similarly identified in mice. Mice are used to model of human aging and many motor disorders, both somatic and central nervous system in orgin. For all of these models, a full assessment of their motor deficients must include specific test of strength.

The present invention demonstrated a statistically significant dramatic increase in clinical fraility tests performed including, Kondziela's inverted screen tests (Latency of falling time, mobility time, four fingers grip), weight test (forelimb grip-holding time), and Open Field Maze (OFM) tests (total distance travel, total Rearing number tests, Supporting Rearing Number, Unsupporting Rearing Number) in groups of 2-year-old mice receiving memantine compared to not receiving group of mice (FIGS. 27A-K).

As mentioned above, many biomarkers related to aging have been identified in human. Importantly, the grip tests performed on humans has been defined as a highly predicitive indicator that can show disabilities, diseases and deaths that may occur due to aging (Iconaru et al 2018; Sousa-Santos and Amaral 2017).

Open Field Maze (OFM). The OFM is one of the most widely used platforms which allow the researcher to measure behaviors ranging from overall locomotor activity to anxiety-related emotional behaviors.

In addition to locomotor activities, the OFM remains one of the most widely applied techniques in rodent behavioral research.

As mentioned above, total ambulatory distance test shows the anxiety state of the mouse in addition to showing locomotor activity. Another test that shows anxiety in mice and is included in OFM tests is Thigmotaxis. This test consist of different measurements such as Central zone time, Peripheral zone time, Central escape latency, Central latency time, and Central zone entrance in the OFM.

In next experiment, when observing the 5-minute movements of the mice in the regions within the OFM, it was determined that mice receiving memantine remained in the central region 4 times more than mice not receiving memantine (P=0.01) (FIG. 27G).

In addition it was determined that mice not receiving memantine remained in the peripheral regions more than mice receiving memantine (p=0.01) (FIG. 27H). The Central escape latency, Central latency time, and Central zone entrance studies, which are other studies of OFM, were also conducted. Although the means of all them differed at least twice, the p value was determined to be greater than 0.05 in all three studies (P values are 0.4, 0.07, and 0.08, respectively).

Many biomarkers related to aging have been identified in human. But, perhaps more importantly than that, locomotor activity is intricately intertwined with complex human activities, such as: Learning, and Motivation. Indeed findings of this invention also shows less anxiety determined by OFM tests in mice receiving memantine than mice not receiving memantine.

Therefore, the present invention encompasses the finding that activation of proteasome by NMDAR blocker, through increased levels and/or increased activity, can provide effective treatment for, and even prophylaxis of, age related emotional anxiety in addition to providing improvement in locomotor activities.

In above studies, we have shown that memantine increase the life span of C. elegans, as well as increase their muscle strength. In mouse studies, we have shown that memantine phenotypically increases muscle strength, decreases amyloid formation (FIG. 29A-C), decreases melanin and lipofuscin deposition (FIGS. 30A and B), fat deposition (FIG. 31A-D), and decreases oxidation of proteins in tissues (FIG. 28), which are markers of the aging, dramatically, we did not investigate the direct effect of memantine on mouse lifespan.

The most important study supporting our invention is the study which is published and patented by Anthony FUTERMAN, Andres David KLEIN showing that memantine increase life span of 4 different strains of mice. The study shows that the increasing of life occurs between 2 and 5 times, depending on the mouse strain (FIGS. 32A-C). In their study, they created Gaucher disease (GD) on 15 inbred mouse strains from diverse phylogenetic origins by inhibition of acid beta-glucosidase (GCase) using the conduritol B-epoxide (CBE). They also demonstrated a correlation between the amount of CBE injected into mice and levels of accumulation of the Gcase substrates, glucosylceramide (GlcCer) and the glycosphingolipids (GSLs), and show that disease pathology, indicated by altered levels of pathological markers, depends on both the levels of accumulated lipids and the time at which their accumulation begins.

They also showed that locomotor activities were improved significantly in mice receiving memantine (FIG. 32D) similar to the findings of this invention.

GD, one of the most common lysosomal storage disorders (LSDs), is caused by mutations in GBA1, the gene encoding the lysosomal hydrolase, GCase. The resulting GCase deficiency causes accumulation of the GSLs, GlcCer and its deacylated forms, within the lysosomes of cells.

Accumulating evidence indicates that GlcCer and Glc-Sph accumulation is directly related to aging. The recently discovered that haploinsufficiency of the GBA gene, leading to a reduction in GCase activity, is one of the most common genetic risk factors for PD. Furthermore, GCase activity is also reduced in brain regions of sporadic PD patients, with a corresponding accumulation of its glycosphingolipid (GSL) substrates.

It is demonstrated that aging in the mouse brain results in a similar lipid profile to that seen in the aging human brain and in PD, with elevated levels of GlcCer, Glc-Sph, lactosylceramide (LacCer). Parallel biochemical analyses revealed a change in lipid metabolism in the aging brain may precede or may be part of abnormal protein handling and may accelerate PD pathophysiological processes in vulnerable neurons in PD and other age-related neurodegenerative disorders.

In addition, the fact that ceramide causes an increase in ROS and increases oxidative stress in many mammalian cells and animal models, directly related to aging, are other important evidences showing the relationship of GCase substrate elevation with aging.

Hereof, findings in mice with GCase inhibition, such as accumulation of insoluble α-synuclein aggregates in the substantia nigra; altered levels of proteins involved in the autophagy/lysosomal system, and widespread neuroinflammation, up-regulation of complement C1q, abnormalities in synaptic, axonal transport and cytoskeletal proteins, and neurodegeneration, can be shown as more direct evidences indicating the relationship of of Gcase substrates elevation with aging.

In addition, significant reductions of mitochondrial adenosine triphosphate production and oxygen consumption (28-40%) were detected in Neuronopathic Gaucher disease (nGD) brains and in CBE-treated neural cells. These studies implicate defective GCase function and GlcCer/Glc-Sph accumulation as risk factors for mitochondrial dysfunction and the multi-proteinopathies (a-synuclein-, APP- and Ab-aggregates) in nGD.

nGD has histopathological features that include CNS pro-inflammatory responses and are associated with astrogliosis, microgliosis and neuronal degeneration in affected humans and mice. Neurodegenerative lesions are often complex with progressive accumulation of protein aggregates (proteinopathies), e.g. a-synuclein, ubiquitin, amyloid precursor protein (APP), b-amyloid (Ab), tau and others in Parkinson disease, Alzheimer disease and Huntington disease. In addition to Gaucher disease, other LSDs also exhibit some accumulations of these proteins, e.g. Tay-Sachs and Sandhoff diseases and Niemann-Pick disease Type C. Recently, clinical and genetic studies demonstrated a-synuclein and ubiquitin protein aggregates in the brains of Gaucher disease patients and mouse models, which typically are present in many non-LSD neurodegenerative diseases. Therefore, Depletion of GCase polypeptide compromises protein degradation capacity and increases a-synuclein levels in neurons

Taken together, these studies suggest that multiple proteinopathies are pathophysiologic features of nGD.

These data are important for the popular view that proteinopathy is the only trigger for aging related disease including neuronal demise in PD because altered GSL homeostasis may parallel or even precede changes in protein aggregation.

Supporting this it was shown that C2-ceramide and C6-ceramide increase senescence as revealed by increased the amount of p53, and p21 which are important members of the the MDM2-p53-p21Cip1 pro-survival pathway of mammalian cells.

Depending on the context, autophagy lysosome pathway and the proteasome share common substrates as well as regulatory factors. Both systems intersect and communicate at multiple points to coordinate and balance their actions in proteostasis and homeostasis of organelles.

The most important concern that may occur as consequence of decreased protesome activity is the accumulation of potentially toxic un/misfolded proteins as well as protein aggregates. The proteasome impairment will eventually affect the vital function of cytosolic processes including autophagy and all other organelles due to the accumulation of unfolded and damaged proteins.

Futerman et. al. showed that by using memantine in all mice with GD symptoms, their lifespan extended and their motor coordination improved significantly. On the other hand, they showed that memantine had no effect on correction of Gcase inhibition, which led to the emergence of symptoms of GD.

Therefore, in fact, Futerman et al. disrupted protein homeostasis network by inhibiting protein degredation capacity by inhibiting GCase actually provided premature aging associated with interferes with crucial signaling pathways including autophagy-lysosome pathway and proteasome.

Unitary Theory of Fundamental Aging Processes hypothesizes that fundamental ageing processes include: (1) chronic low grade ‘sterile’ (absence of bacteria, fungi, etc.) inflammation often accompanied by fibrosis, 2) macromolecular dysfunction (e.g. protein misfolding and aggregation, decreased proteasome activity, DNA damage, telomere uncapping, increased advanced glycation end-products [AGEs], lipotoxicity and accumulation of bioactive lipids) and organelle dysfunction (altered nuclear membranes related to deficient lamin B, mitochondrial dysfunction leading to reduced fatty acid metabolism, higher glucose utilization, depletion of NAD+ and increased ROS generation, etc.), 3) stem, progenitor and immune cell dysfunction (including altered proliferative capacity and dysdifferentiation with failure to develop into functional mature cells, declines in ‘geroprotective’ factors [e.g. a-Klotho], contributing to stem and progenitor cell dysfunction) and 4) cellular senescence may be interlinked. It was hypothesed that targeting any one fundamental ageing process (indicated above) genetically or with drugs should affect many or perhaps all of the rest (Austad 2016).

Therefore, as stated in this invention, the homeostasis of proteins provided by memantine by activating the proteasome plays a major role in the preventation of many or perhaps all of the fundamental ageing process. In this way, premature aging and even premature death of mice in the clinical picture caused by inhibiting GCase by Futerman et al. was prevented (see, United States, Patent, Futerman et al, Application Pub. No.: US 2017/0273917 A1 and date Sep. 28, 2017 and Klein et al. 2016)

Similarly to the results of Futerman et al, present invention demonstrated a statistically significant dramatic increase in clinical fraility tests performed including, Falling time, mobility time, four fingers grip, forelimb grip, total distance travel, Total Rearing number tests, in groups of 2-year-old mice receiving memantine compared to not receiving group of mice.

Continuous turnover of intracellular proteins is essential for the maintenance of cellular homeostasis and for the regulation of multiple cellular functions. All intracellular proteins undergo continuous synthesis and degradation. This constant protein turnover, among other functions, helps reduce, to a minimum, the time a particular protein is exposed to the hazardous cellular environment, and consequently, the probability of being damaged or altered. At a first sight, this constant renewal of cellular components before they lose functionality may appear a tremendous waste of cellular resources. However, it is well justified considering the detrimental consequences that the accumulation of damaged intracellular components has on cell function and survival. Furthermore, protein degradation rather than mere destruction is indeed a recycling process, as the constituent amino acids of the degraded protein are reutilized for the synthesis of new proteins. The rates at which different proteins are synthesized and degraded inside cells are different and can change in response to different stimuli or under different conditions. This balance between protein synthesis and degradation also allows cells to rapidly modify intracellular levels of proteins to adapt to changes in the extracellular environment. Proper protein degradation is also essential for cell survival under conditions resulting in extensive cellular damage. In fact, activation of the intracellular proteolytic systems occurs frequently as part of the cellular response to stress. In this role as ‘quality control’ systems, the proteolytic systems are assisted by 20S proteasome, which ultimately determine the fate of the oxidatively damaged/unfolded protein.

It has been shown that maintaining proteostasis is the defense mechanism against accumulating proteotoxic stresses induced by oxidative damage and thus prevents the ageing and aging associated diseases and increase life span.

The ubiquitin-proteasome system is now accepted as the main intracellular proteolytic system and malfunctioning of these this system can contribute to different aspects of the phenotype of aging and to the pathogenesis of some age-related diseases.

This age-related decline in proteolytic activity has been observed in almost all organisms analyzed, and specific defects in the different proteolytic systems with age have been reported. Keeping in mind the myriad of intracellular functions in which protein degradation participates, it is not surprising that the consequences of the age-related alterations in the proteolytic systems are widespread and contribute to a broad variety of pathologies.

Many literature exists that on increased amounts of oxidized proteins in skeletal muscle, heart, skin, liver, lymphocytes and other tissues of various animal species.

Cardiovascular diseases, disorders, and/or conditions are a leading cause of deaths worldwide. By the time that cardiovascular heart problems are usually detected, the disease is usually quite advanced, having progressed for decades, and often too advanced to allow successful prevention of major permanent disability.

In general, cardiovascular disease may be a disease which involves the heart and/or blood vessels, arteries, and occasionally veins. In some embodiments, the disease is a vascular disease. Exemplary particular proteinopathic cardiovascular diseases, disorders, and/or conditions may include myocardial ischemia, myocardial infarction, coronary heart disease, an acute coronary symptom, unstable angina pectoris or stable angina pectoris, stroke, ischemic stroke, vascular hyperplasia, cardiac hypertrophy, congestive heart failure, restenosis, arteriosclerosis, atherosclerosis, vasculitis, polyarteritis nodosa, myocarditis, hypertension, inflammation or autoimmune disease associated atherosclerosis or restenosis. In addition the irreversible loss of cardiomyocytes due to oxidative stress is the main cause of heart dysfunction following ischemia/repeifusion injury and ageing-induced cardiomyopathy (Jian et al. 2005; Giordano 2005; Eltzschig and Eckle 2011).

Therefore, the present invention provides compositions and methods which prevents heart diseases mentioned above by maintaining and/or improving the performance of the heart muscle, and is effective in ameliorating onset and/or progression of the heart dysfunction following ischemia/repeifusion injury and ageing-induced cardiomyopathy.

Aging is a major determinant for the high prevalence of metabolic syndrome. One major threat with the ever-rising aging population with metabolic syndrome is the prevalence of aging-associated cardiovascular morbidity and mortality such as atherosclerotic vascular dysfunction. Ample of clinical evidence has suggested much higher mortality of myocardial infarction or stroke in patients premature aging.

Although a number of theories have been articulated for the relationship between premature aging and metabolic syndrome, nowadays, the accepted view is the impairment of protein homeostasis and protein quality control. The accumulating proteotoxic stresses induced by oxidative damage and related decrease in proteasome activities are the main cause protein homeostasis and protein quality control.

Therefore, in another embodiment the present invention provides a pharmaceutical composition comprising at least one NMDA receptor antagonist or a pharmaceutically acceptable salt, hydrate or pharmaceutically active enantiomer thereof, for use in preventing the metabolic syndrome by regulating protein homeostasis and protein quality control.

The changes in lipid metabolism with age that contribute to increased plasma free fatty acid concentrations or increased nonoxidative disposal cause may contribute to various metabolic derangements including, obesity, accumulation of total and central body fat, insulin resistance, inflammation, loss of muscle mass and strength. Collectively, changes in lipid metabolism with age that contribute to increased plasma free fatty acid concentrations or increased nonoxidative disposal contribute to increased risk for the development of diabetes and cardiovascular disease. A reduction in the size and/or oxidative capacity of the metabolically-active muscle mass is probably a more likely determinant of reduced fat oxidation. Therefore, the preservation of muscle mass and activity during aging prevents the metabolic syndrome by preventing changes in lipid metabolism.

In addition, muscle mass is the processing center of glucose metabolism in the whole body, participating in 80% of glucose uptake and treatment in the whole body. Imbalance of muscle mass metabolism strongly influences glucose homeostasis and insulin sensitivity throughout the body, which can cause SkM inflammation, induce oxidative stress muscle mass, and damage muscle mass health. In addition to participating in insulin-related glucose metabolism, muscle mass also participates in multiple metabolic pathways of the body, including lipid metabolism.

As indicated above present invention demonstrated that locomotor activities of both C. elegans and mice treated with NMDAR inhibitor were significantly increased compared to the controls (FIGS. 25C, 27A-K). In addition, present invention demonstrated that amount of lipid droplets in muscle tissues of both C. elegans and mice treated with NMDAR inhibitor was significantly less than in the non-NMDAR inhibitor-treated controls (FIGS. 25D, E; 32A-E).

Thus, the present invention provides, in one aspect, a pharmaceutical composition comprising at least one NMDA receptor antagonist or a pharmaceutically acceptable salt, hydrate or pharmaceutically active enantiomer thereof, for use in preventing the metabolic syndrome by increasing muscle mass and activity and accordingly regulates lipid metabolism.

In addition, the present invention provides, in one aspect, a pharmaceutical composition comprising at least one NMDA receptor antagonist or a pharmaceutically acceptable salt, hydrate or pharmaceutically active enantiomer thereof, for use in preventing the obesity and related diseases which occur in conjunction with metabolic syndrome caused by the changes in lipid metabolism with age.

Therefore, the present invention encompasses the finding that activation of proteasome by NMDAR blocker, through increased levels and/or increased activity, is useful in the prevention, amelioration and/or treatment of disorders of the metabolism influencing body weight, in particular in the treatment of obese subjects, in particular in the treatment of human subjects.

In accordance with this invention it is also envisaged that NMDA receptor antagonists, in particular of memantine, a pharmaceutically acceptable salt or a prodrug of NMDA receptor antagonists is employed in the medical intervention of secondary disorders related to a (pathological) increase of body weight. These ‘secondary disorders’ may comprise, but are not limited to diabetes type 2, high blood pressure (hypertension), cardiovascular diseases, cancer, problems with sexual function and disorder of the muscular or bone system.

With an increase in age, the muscle mass declines at a rate of about 1-2% per year greatly increasing the risk of muscle disease including (i) mitochondrial dysfunction, which causes various muscle pathologies such as sarcopenia and muscular dystrophy, (ii): inflammatory myopathy caused by inflammation and oxidative stress, such as dermatomyositis, polymyositis, necrotizing autoimmune myositis, and sporadic inclusion body myositis, and (iii) chronic diseases that cause SkM damage, such as type 2 diabetes, obesity, chronic kidney disease, and chronic obstructive pulmonary disease. These syndromes often lead to a decline in the quality of life of patients over time and increase patient mortality.

Thus, the present invention provides a method for use in preventing and/or treating muscle diseases mentioned above.

The present invention, in another aspect, provides compositions and methods which maintain and/or decrease the aging of skin and hair by decreasing accumulation of oxidized protein, lipids and inflammation. Therefore, present invention according to an aspect of some embodiments of the present invention there is provided an NMDA receptor antagonist for use in detoxifying the cells with oxidatively damaged proteins and lipids by inducing the activity of the 20S proteasome complex which is usable in cosmetics including hair whitening, skin wrinkles and age spots on the skin.

Furthermore, the present invention provides compositions and methods which Maintain and increase the stemness characteristics. The term “stemness” is a property of a cell-namely, the property of having a certain gene or genes whose expression makes possible both the potential for self-renewal and the potential for multilineage differentiation. The Stemness, accepted, is ‘the highest degree of plasticity of a cell, within the repertoire of cell types present in the organism’.

It was shown by Kapetanou et al. that the proteasome activation enhances stemness and lifespan of human mesenchymal stem cells (Kapetanou etal, 2017).

An indicator of the stem cell differentiation is the determination of the amount of stemnnes markers including, cd44, cd73, and cd90 in advanced cell passages (Ali et al. 2015; Gonzilez-Garza et al 2018, Yoo et al. 2020). Therefore, in this invention, these markers in memantine treated and non-treated stem cells were investigated. The amount of cd44, cd73, and cd90 have increased on the 14th day according to the control cells (non-passaged) and this increase in markers in memantine treated cell is statistically significantly less than the cell not memantine treated, on day 14 (FIG. 36A-C). Another indicator of the stem cell differentiation is the determination of the amounts of lipid droplet in the cell. Fourteen-day cell passage is caused to increase lipid in the cell. It was observed that memantine treatment has prevented the amount of lipid droplet (FIG. 36D). In addition, present invention increase the lifespan of human mesenchymal stem cells. FIG. 36E shows the MTT test of the cells on the day 21 (P<0,034).

Therefore, in another embodiment, the present invention provides compositions and methods which maintain and increase the stemness characteristics which is the potential for self-renewal and for multilineage differentiation and lineage reprogramming of stem cells.

With an increase in age, the physiological activity of muscle stem cells decreases and the regeneration capacity of SkM declines and the efficiency of muscle repair decreases, and this is accompanied by a decrease quality and strength of muscles, and irreversible muscle loss which seriously affect the quality of life of the elderly.

Therefore, the present invention provides compositions and methods which maintain and increase the stemness characteristics and prevent a decrease quality and strength of muscles, irreversible muscle loss, and muscle diseases which seriously affect the quality of life of the elderly by increasing proteasome activity.

The effects of the present invention is also not limited to muscle tissue, but also the invention provides compositions and methods which maintain and increase the stemness characteristics and prevent aging and associated diseases by ensuring the continuation of the regenerative character of all tissues including neurons.

Ocular pathologies of high incidence, such as age-related macular degeneration, cataracts, glaucoma, and diabetic retinopathy which are of multifactorial origin and are associated with genetic, environmental factors, age, and oxidative stress, among others. oxidative stress is one of the most influential in ocular diseases. The most important concern that may occur as consequence of decreased protesome activity is the accumulation of potentially toxic un/misfolded proteins as well as protein aggregates. The proteasome impairment will eventually affect the vital function of cytosolic processes including autophagy and all other organelles due to the accumulation of unfolded and damaged proteins. Alteration of the normal functioning of autophagy processes can interrupt organelle turnover, leading to the accumulation of cellular debris and causing physiological dysfunction of the eye.

Ocular pathologies of high incidence, such as age-related macular degeneration (AMD) is characterized by accumulation of extracellular deposits, namely drusen, along with progressive degeneration of photoreceptors and adjacent tissues. The protein composition of drusen includes apolipoproteins and oxidized proteins. Chronic inflammation, lipid deposition, oxidative stress and impaired extracellular matrix maintenance are strongly implicated in AMD pathogenesis.

According to an aspect of some embodiments of the present invention there is provided an NMDA receptor antagonist for use in treating or preventing ocular pathologies related with impaired protein organel turnover by increasing the proteasome activity and, accordingly, it acts by regulating autophagy-lysosome pathway (Fleckenstein et al. 2021; Fernandez-Albarral et al. 2021; Blasiak et al. 2021).

According to an aspect of some embodiments of the present invention there is provided an NMDA receptor antagonist for use in treating viral enfections by degredading proteins of viruses, such as HepC core protein. Another subject of this invention is HIV because proteasome inhibitors are known to activate the latent HIV (Cary and Peterlin, 2020; Timilsina et al. 2019).

As mentioned present invention also showed that NMDAR inhibitors decrease in carbonylated and carbomylated proteins which are responsible for the structural changes lead to the exposure of abdominally destructured areas on the protein surface generating neoantigens, which are responsible for autoimmune reactions (FIG. 28) (Jaisson et al. 2018; Shi et al. 2011).

The accumulating evidences show that proteasome dysfunction is connected with (auto)inflammation and autoimmunity (Brehm and Krüger, 2015). The autoimmune responses arised against protein aggregations caused by oxidatively damaged and/or misfolded proteins often accompanied by altered cytokine patterns, such as diabetes or Sjögren syndrome.

Importantly one of the crucial problem that arise in the functional deficiencies of proteasome activity is the increase in the functional susceptibility to infections, especially viral, late recovery, and the emergence of autoimmunity after infections. For example; LMP7 (PSMB8) knockout mice are physically undistinguishable from wild-type mice, but they are more susceptible to some infections, and pathogen clearance is prolonged or inadequate. In particular, an example for the CNS is that the immunoproteasome, a special isoform of the 20S proteasome deficiency was found to result in significantly increased clinical scores by studying autoimmune encephalomyelitis (EAE) in a mouse model. The immunoproteasome (IP) deficiency was further associated with severe heart muscle injury with large inflammatory lesions and severe myocardial tissue damage in a mouse model for Coxsackie virus B3-induced myocarditis. This protective role is also evidenced by studies showing that IP-deficient mice severely suffer from impaired stress responses, survival, and/or clearance rates upon infection or inflammation or develop severe LPS-induced hepatitis.

Moreover, IP dysfunction is connected with (auto)inflammation and autoimmunity, often accompanied by altered cytokine patterns, such as diabetes or Sjögren syndrome ((Brehm and Krüger, 2015).

Several previously described autoinflammatory syndromes, such as Nakajo-Nishimura syndrome (NNS), joint contractures, muscle atrophy, microcytic anemia and panniculitis-induced lipodystrophy (JMP) syndrome, Japanese autoinflammatory syndrome with lipodystrophy (JASL), and chronic atypical neutrophilic dermatosis with lipodystrophy and elevated temperature (CANDLE), were are now classified as a spectrum of diseases named proteasome-associated autoinflammatory syndrome (PRAAS). The multiple inflammatory developments include nonspecific lymphadenopathy, hepatosplenomegaly, autoimmune hemolytic anemia, hypertriglyceridemia, and lipodystrophy.

The most common known cause of the syndrome are mutations in the Proteasome Subunits, including PSMB8 (encodes β1i), PSMA3 (encodes α7), PSMB4 (encodes β7), PSMB9 (encodes β1i), genes that codes for proteasomes that in turn break down other proteins. The reported mutations have different impacts on the proteasome by modulation of gene expression, subunit folding and maturation, assembly of the core complex, and/or structural alterations of the proteolytic pocket, but all result in reduced proteasome activity which is insufficient to cope with a higher load of damaged proteins, and results in proteins not being degraded and oxidative proteins building up in cellular tissues, such as intra mitochondrial paracrystalline inclusions and cytoplasmatic and myeloid bodies, indicating accumulation of damaged/aggregated proteins eventually leading to apoptosis, especially in muscle and fat cells. These findings strongly suggest that the disturbance of the overall proteasome activity and capacity in response to environmental stress is crucial (Brehm and Krüger, 2015).

In certain embodiments, proteinopathic inflammatory diseases, disorders, and/or conditions may include one or more of inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, irritable bowel syndrome, ulcerative colitis, glomerulonephritis, dermatomyositis, scleroderma, vasculitis, allergic disorders including asthma such as bronchial, allergic, intrinsic, extrinsic and dust asthma, particularly chronic or inveterate asthma (e.g. late asthma airways hyper-responsiveness) and bronchitis, chronic obstructive pulmonary disease, multiple sclerosis, rheumatoid arthritis, disorders of the gastrointestinal tract, including, without limitation, Coeliac disease, proctitis, eosinophilic gastro-enteritis, mastocytosis, pancreatitis, Crohn's disease, ulcerative colitis, food-related allergies which have effects remote from the gut, e.g. migraine, rhinitis and eczema. Conditions characterised by inflammation of the nasal mucus membrane, including acute rhinitis, allergic, atrophic thinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung and idiopathic interstitial pneumonia, acute pancreatitis, chronic pancreatitis, and adult respiratory distress syndrome, and/or acute inflammatory responses (such as acute respiratory distress syndrome and ischemia/reperfusion injury).

The other examples of immune-mediated responses and diseases include, rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xenotransplants, etc.; treatment of graft-versus-host disease, autoimmune diseases, such as rheumatoid arthritis, systemic lupus erythematosus, thyroiditis, Hashimoto's thyroiditis, multiple sclerosis, myasthenia gravis, type I diabetes uveitis, juvenile-onset or recent-onset diabetes mellitus, uveitis, Graves' disease, psoriasis, atopic dermatitis, Crohn's disease, ulcerative colitis, vasculitis, auto-antibody mediated diseases, aplastic anemia, Evan's syndrome, autoimmune hemolytic anemia, and the like; and further to treatment of infectious diseases causing aberrant immune response and/or activation, such as traumatic or pathogen induced immune dysregulation, including for example, that which are caused by hepatitis B and C infections, HIV, Staphylococcus aureus infection, viral encephalitis, sepsis, parasitic diseases wherein damage is induced by an inflammatory response (e.g., leprosy). Other immune-mediated responses and diseases relate to graft vs host disease (especially with allogenic cells), rheumatoid arthritis, systemic lupus erythematosus, psoriasis, atopic dermatitis, Crohn's disease, ulcerative colitis and/or multiple sclerosis.

Examples also include, diseases caused or worsened by the host's own immune response. For example, autoimmune diseases such as multiple sclerosis, lupus erythematosus, psoriasis, pulmonary fibrosis, and rheumatoid arthritis and diseases in which the immune response contributes to pathogenesis such as atherosclerosis, inflammatory diseases, osteomyelitis, ulcerative colitis, Crohn's disease, and graft versus host disease (GVHD) often resulting in organ transplant rejection. Additional exemplary inflammatory disease states include fibromyalgia, osteoarthritis, sarcoidosis, systemic sclerosis, Sjogren's syndrome, inflammations of the skin (e.g.., psoriasis), glomerulonephritis, proliferative retinopathy, restenosis, and chronic inflammations.

The present invention provides an enhancement of proteolytic capacity by increasing proteasome activity by using of at least one NMDA receptor antagonist or a pharmaceutically acceptable salt, hydrate or pharmaceutically active enantiomer that ameliorate onset and/or progression and even treatment of the autoimmune responses arised against protein aggregations caused by oxidatively damaged and/or misfolded proteins.

The present invention provides methods and compositions related to diverse group of neurodegenerative proteinopathies that are linked to D-serin increase.

D-serine (DS), an endogenous D-amino acid, is produced by serine racemase (SR) in glial cells and neuronsl. SR converts the free form of L-serine into DS. The free DS functions as a co-agonist of NMDA receptor in the brain, retina, keratinocytes and in chondrocytes. Incorporation of D-serine causes significant changes in the functional properties of many proteins that are detrimental to humans. Racemization of serine residues is known to occur in beta-amyloid senile plaques of Alzheimer's disease (AD), human lens protein α-crystallin, human myelin basic protein, osteoarthritic articular and meniscal cartilages, the funnel-web spider toxin, egg ovalbumin and in skin. In addition to serine isomerization, SR also performs α, β-elimination reaction with both L-serine and D-serine to produce pyruvate and ammonia (Rani et al. 2020).

One of the main features of the NMDA receptor is that its activation requires simultaneous binding of its agonist glutamate and co-agonist glycine. DS binds to NMDA receptor at the glycine site with three-fold higher affinity than glycine and the efficacy of DS to activate the NMDA receptor is 10 times higher in comparison to glycine. Hyperactivation of the NMDA receptor takes place in excess of DS.

The hyperactivity of the NMDA receptor is involved in many neurological diseases such as Alzheimer's disease, amyotrophic lateral sclerosis, epilepsy, neuropathic pain, and cerebral ischemia.

Therefore, the inhibition of SR is another way to regulate the NMDA receptor transmission and is the only way to prevent serine isomerization occurring in proteins in certain pathological conditions including the AD. SR inhibition is proven to be neuro-protective in many situations.

Therefore, in another aspect, present invention demonstrates that increased protesome activity in the presence of NMDAR blocker decrease serin racemesa (FIG. 4B) which decrease L-to-D-serin conversion, which will be effective in preventing or treating diseases of cerebral ischemia, traumatic brain injury, peripheral neuropathy, cerebral ischemia, choroidal neovascularization, Hyperactivity brain syndrome, Long-term Sequelaes of concussion, Seizure, retinal ganglion cell loss in diabetics, chronic social defeat stress, and other neurodegeneratif diseases.

And importantly, in another aspect, present invention provides compositions and methods which act as neuro-protective in neurological diseases.

The term “neuro-protective activity” as used herein refers to the effects of reducing or ameliorating nervous insult, and protecting or reviving neuronal cells that have suffered nervous insult. As used herein, the term “nervous insult” refers to any damage to neuronal cell or tissue resulting from various causes such as metabolic, toxic, neurotoxic and chemical causes. Also it refers to aging, which is the process of accumulating all of these causes (Darric et al. 2019).

Recent evidences show that astrocytic d-serine promote neuronal degeneration in Alzheimer's disease. The present invention provides compositions and methods which reduce the toxic effects of astrocytes by decreasing serin racemesa. Therefore, present inventions further provide a decrease in microvascular damage in diabetic retinopathy, synaptic damage after traumatic brain injury, neuronal over-activation, choroidal neovascularization, the pathologic symptoms of Alzheimer's disease, and neurodegeneration (Balu et al 2019).

In another aspect, present invention demonstrates that increased protesome activity in the presence of NMDAR blocker decrease serin racemesa, which decrease L-to-D-serin conversion, which will be effective in preventing or treating schizophrenia and other psychological diseases (Kishi and Iwata 2013).

According to an aspect of some embodiments of the present invention demonstrated that an NMDA receptor antagonist decrease phosphorylated tau (pTau) by increasing the proteasome activation (FIG. 4A). Tau is one of the major components of microtubule (MTN) networks in neurons, and its abnormal phosphorylation and aggregation are closely related to the impairment of axonal transport (Weng and He 2021).

MTNs are crucial for neurons, as the neuronal signaling and protein turnover between cell bodies and axons highly rely on the axonal transport along MTNs. The function of MTNs can be regulated by tau protein. Through binding to the microtubules, tau modulates the stability and dynamics of microtubules. Abnormal phosphorylation of tau interrupts its binding to microtubules and leads to disruption of MTNs. In this sense, the accumulation of p-tau, a salient feature in AD, may impede the normal functioning of MTNs and cause a series of catastrophic cascades (Weng and He 2021).

Tauopathies are neurodegenerative disorders characterized by the presence of filamentous deposits, consisting of hyperphosphorylated tau protein, in neurons and glia. Abnormal tau phosphorylation and deposition in neurons and glial cells is one of the major features in tauopathies.

This term is now used to identify a group of diseases with widespread tau pathology in which tau accumulation appears to be directly associated with pathogenesis. Major neurodegenerative tauopathies includes sporadic and hereditary diseases characterized by filamentous tau deposits in brain and spinal cord. In the majority of tauopathies, glial, and neuronal tau inclusions are the sole or predominant CNS lesions. Exemplary such tauopathies include amytrophic lateral sclerosis (ALS), parkinsonism, argyrophilic grain dementia, diffuse neurofibrillary tangles with calcification, frontotemporal dementia linked to chromosome 17, corticobasal degeneration, Pick's disease, progressive supranuclear palsy, progressive subcortical gliosis, and tangle only dementia.

Additionally, tauopathies characterize a large group of diseases, disorders and conditions in which significant filaments and aggregates of tau protein are found. Exemplary such diseases, disorders, and conditions include sporadic and/or familial Alzheimer's Disease, amyotrophic lateral sclerosis/parkinsonism-dementia complex (ALS-FTDP), argyrophilic grain dementia, dementia pugilistica, diffuse neurofibrillary tangles with calcification, Down syndrome, frontotemporal dementia, parkinsonism linked to chromosome 17 (FTDP-17), Gerstmann-Straussler-Scheinker disease, Hallervorden-Spatz disease, inclusion body myositis, Creutzfeld-Jakob disease (CJD), multiple system atrophy, NiemannPick disease (NPC), Pick's disease, prion protein cerebral amyloid angiopathy, progressive supranuclear palsy (PSP), subacute sclerosing panencephalitis, tangle-predominant Alzheimer's disease, coiticobasal degeneration, (CBD), myotonic dystrophy, non-guanamian motor neuron disease with neurofibrillary tangles, postencephalitic parkinsonism, prion protein cerebral amyloid angiopathy, progressive subcortical gliosis, subacute sclerosing panencephalitis, and tangle-only dementia.

Neurodegenerative diseases where Tau pathology is found in conjunction with other abnormal protein lesions may be considered secondary tauopathies. Examples include AD and certain diseases where prion protein, Bri, or a-synuclein are aggregated. Although tau is probably not the initial pathological factor, tau aggregates contribute to the final degeneration.

Alzheimer's disease (AD) is a neurodegenerative disorder affecting numerous people around the world. It is the most common cause of dementia (Alzheimer's Association, 2020) and the number of its victims continues to expand as the population ages. It has now become the sixth leading cause of death in the United States (Alzheimer's Association, 2020). Although the pathogenesis and treatment of AD are under extensive scrutiny, the field has not achieved great gains yet. Intracellular aggregates primarily consisting of phosphorylated tau (p-tau) are one of the hallmarks in AD (Alzheimer's Association, 2020; Weng and He, 2021). Pathological proteinopathy is also recognized as a subset of the proteinacious lesions detected in neurodegeneration with brain iron accumulate type I, amyotrophic lateral sclerosis/Parkinson's dementia complex of Guam, multiple systems atrophy, and familial AD.

The proteasome system maintains intracellular proteostasis by degrading abnormal or redundant proteins. Ever-mounting evidence suggests that proteasome activity deficits contribute to p-tau accumulation. And increasing proteasome activity attenuates tau pathology (Boselli 2017). Indeed, the present invention demonstrates that increased protesome activity in the presence of NMDAR blocker decrease pTau amount.

Therefore, in one aspect present invention provides compositions and methods which prevent and/or treat diseases mentioned above caused by the tauopathy.

pTau transgenic mice show disrupted mitochondrial dynamics and impaired mitophagy. The studies reveal that aggregation of APP and its cleavage product, Aβ, and pTau leads to deficiencies in autophagy and mitophagy (Bussian et al. 2018). Therefore, in the presence of NMDAR blocker, as demonstrated by this invention, establishment of protein homeostosis should be ensured before the emergence of pathologies such as deficiencies in autophagy and mitophagy by the emergency of the pTau and other cleavage products.

Cognitive impairment and dementia are commonly defined as a progressive decline in cognitive function due to damage or disease in the body beyond what is expected from normal aging. Dementia is described as a loss of mental function, involving problems with memory, reasoning, attention, language, and problem solving. Higher level functions are typically affected first. Dementia interferes with a person's ability to function in normal daily life.

The cognitive impairment or dementia may stem from any etiology. Particular diseases that are associated with cognitive impairment or dementia include, but are not limited to, neurodegenerative diseases, neurological diseases, psychiatric disorders, genetic diseases, infectious diseases, metabolic diseases, cardiovascular diseases, vascular diseases, aging, trauma, malnutrition, childhood diseases, chemotherapy, autoimmune diseases, ocular diseases, and inflammatory diseases.

The present invention provides methods that prevents, reduces or even treats the cognitive impairment and dementia in normal aging and cognitive impairment and dementia stem from any etiology.

The present invention further provides, in another aspect, a finding that NMDAR blocker causes actin turnover by increasing the proteasome activity (FIG. 35). Previously described that actin turnover promotes axon regeneration in the adult CNS (Tedeschi and Dupraz 2019). In another aspect, present invention may provide an increase in axon regeneration. Therefore, the present invention provides compositions and methods which prevent and/or treat neurodegeneration caused by pathological insults including anticancer treatments, cerebral ischemia, traumatic brain injury, peripheral neuropathy, cerebral ischemia, choroidal neovascularization, retinal ganglion cell loss, long-term sequelaes of concussion, decreasing Neurotoxic effects of astrocytes in Alzheimer's disease.

In one aspect present invention provides compositions and methods which prevent and/or treat renal diseases caused by accumulation of oxidatively damaged and/or misfolded proteins.

In another aspect present invention provides compositions and methods which prevent and/or treat renal insufficiencies caused by aging, hyperlipidemia, hypertension, smoking, diabetes, obesity and other factors which cause oxidative damage to proteins (Cauli et al. 2014). Therefore, the present invention ameliorate and/or treat acute and chronic kidney disease.

The unfolded protein response (UPR) is an adaptive cellular program used by eukaryotic cells to cope with protein misfolding stress (Madden et al. 2019; Hsu et al. 2019). The UPR is a pro-survival mechanism triggered by accumulation of unfolded or misfolded proteins in the endoplasmic reticulum (ER), a condition referred to as ER stress. Over the past decade, evidence has emerged supporting a role for the UPR in the establishment and progression of several cancers. In addition several literatures connects cancer chemotherapy resistance mechanisms and activation of the UPR (Madden et al. 2019; Hsu et al. 2019).

The present invention provides methods that prevents, reduces or even treats the cell proliferative diseases by reducing or preventing the unfolded protein accumulation and subsequently UPR.

In general, cell proliferative disorders, diseases, and/or conditions encompass a variety of conditions characterized by aberrant cell growth, preferably abnormally increased cellular proliferation. For example, proteionopathic cell proliferative diseases, disorders, and/or conditions include, but are not limited to, cancer, immune-mediated responses and diseases (e.g., transplant rejection, graft vs host disease, immune reaction to gene therapy, autoimmune diseases, pathogen-induced immune dysregulation, etc.), certain circulatory diseases, and certain neurodegenerative diseases.

According to the invention, the term “cell proliferative disorders” also refers to cancer diseases. The terms “cancer disease” or “cancer” (medical term: malignant neoplasm) refer to a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor, i.e. a swelling or lesion formed by an abnormal growth of cells (called neoplastic cells or tumor cells), but some, like leukemia, do not. Examples of cancers include, but are not limited to, carcinoma, leukemias and lymphomas such as cutaneous T-cell lymphomas (CTCL), peripheral T-cell lymphomas, lymphomas associated with human T-cell lymphotropic virus (HTLV) such as adult T-cell leukemia/lymphoma (ATLL), B-cell lymphoma, acute lymphocytic leukemia, acute nonlymphocytic leukemias, chronic lymphocytic leukemia, chronic myelogenous leukemia, acute myelogenous leukemia, Hodgkin's disease, non-Hodgkin's lymphomas, multiple myeloma, myelodysplastic syndrome, mesothelioma, common solid tumors of adults such as head and neck cancers (e.g., oral, laryngeal and esophageal), genitourinary cancers (e.g., prostate, bladder, renal, uterine, ovarian, testicular, rectal and colon), lung cancer, breast cancer, liver cancer, colon cancer, cancer of the small intestine, pancreatic cancer, melanoma and other skin cancers, stomach cancer, neoplasms of the central nervous system (CNS), neuroectodermal cancer, spinal axis tumors, glioma, meningioma, and pituitary adenoma liver cancer and thyroid cancer, and/or childhood solid tumors such as brain tumors, neuroblastoma, retinoblastoma, Wilms' tumor, bone tumors, and soft-tissue sarcomas. The term “cancer” according to the invention also comprises cancer metastases.

Present invention, in another aspect, provides a method for preventing chemotherapy resistance mechanisms caused by UPR.

The present invention further provides, in another aspect, a method for treating different forms of cancer, in which protein homeostasis network is disturbed including increased protein synthesis, and misfolded protein production, accumulation of both misfolded protein aggregates, as well as apoptosis signaling proteins in cancer cells. This can change the sensitivity of cancer cells to antineoplastic drugs (Madden et al. 2019; Hsu et al. 2019).

Serine racemase (SR) converts the free form of L-serine into D-serine (DS) in the cells. The DS functions as a co-agonist of NMDAR. The over-activation of the NMDA receptor leads to many neurological disorders like stroke, amyotrophic lateral sclerosis, and AD. NMDAR inhibitors (ketamin) degraded Serine racemase (SR) by activating proteasome (FIG. 4A, B).

Therefore, in another aspect, present invention provides a method for treating colorectal cancer by decreasing Serine racemase which enhances growth of colorectal cancer by producing pyruvate from serine (Ohshima etal., 2020).

Previous studies have shown that inhibition of proteasome activity inhibits telomerase activity (Shalem-Cohavi etal, 2019). The shortening of telomere length is one of metabolic derangements compromise the normal aging process (such as loss of muscle mass and strength).

Therefore, in another aspect, the present invention provides that NMDA receptor antagonists increase telomerase activity by increasing the activity of proteasome. Consequently, present invention provides a method that ameliorate/correct metabolic derangements and this compromise the normal aging process. Present invention further provides that NMDA receptor antagonists delays cell senescence by increasing telomerase activity which provides prolongation of life (Shalem-Cohavi et al. 2019).

### NMDR Blockers as a Senolytic Drugs

Mammalian cells have the exceptional ability to adapt to perturbations in the extracellular and intracellular environments. Perturbations in a cell's microenvironment promote the activation of metabolic and molecular changes to ensure cell survival. Despite these adaptive mechanisms, chronic or severe, irreparable damage will terminate the damaged cell to preserve an organism's life. The termination of a damaged cell refers to the end of the normal physiological status of the cell. This can happen through cellular division inactivation, a state known as senescence, or by the elimination of the damaged cells, a process known as regulated cell death. Even if the mechanism is not completely understood, the cell cycle regulator p53 has a key role in controlling the decision to activate pro-apoptotic regulators in response to severe damage or to regulate p21Cip1 transcription to induce senescence in response to a milder but still damaging insult. Therefore, when the damage is severe enough but is not lethal, cells switch to a permanent, non-proliferating state. Intra and extracellular signals that can contribute to cells' entering the senescent cell fate mainly include signals related to tissue or cellular damage and/or cancer development. These include protein aggregates, misfolded proteins, failed protein removal through decreased proteasome activity, presence of AGEs due to the reaction of reducing sugars with amino groups in proteins (e.g. Haemoglobin Alc is an AGE), saturated lipids and other bioactive lipids (bradykines, certain prostaglandins, etc.), reactive metabolites (e.g. ROS, hypoxia or hyperoxia), DNA damage, telomeric uncapping or dysfunction, exposure to extracellular DNA, oncogene activation, replicative stress or inducers of proliferation (such as growth hormone/IGF-1), inflammatory cytokines (e.g. TNFa). Therefore, cellular senescence is a complex stress response that causes an essentially irreversible arrest of cell proliferation and development of a multicomponent senescence-associated secretory phenotype (SASP).

The SASP consists of a myriad of cytokines, chemokines, growth factors, and proteases that initiate inflammation, wound healing, and growth responses in nearby cells. In young healthy tissues, the SASP is typically transient and tends to contribute to the preservation or restoration of tissue homeostasis. However, senescent cells increase with age, and a chronic SASP is known or suspected to be a key driver of many pathological hallmarks of aging, including chronic inflammation, tumorigenesis, and impaired stem cell renewal (Saez-Atienzar and Masliah 2020).

These inducers activate one or more senescence-promoting transcription factor cascades, in some cases involving p16INK4a-retinoblastoma protein (Rb), in others, p53 and p21CIP1, both of these pathways, or other pathways.

Data from several laboratories, strongly support the idea that senescent cells and the SASP drive multiple age-related phenotypes and pathologies, including atherosclerosis, osteoarthritis, cancer metastasis, cardiac dysfunction, myeloid skewing, kidney dysfunction, and overall decrements in health span.

SASP is one of the three main features of senescent cells, the other two features being arrested cell growth, and resistance to apoptosis. Senescent cells are highly metabolically active, producing large amounts of SASP, which is why senescent cells consisting of only 2% or 3% of tissue cells can be a major cause of aging-associated diseases. SASP factors cause non-senescent cells to become senescent. SASP factors induce insulin resistance. SASP disrupts normal tissue function by producing chronic inflammation, induction of fibrosis and inhibition of stem cells. Chronic inflammation associated with aging has been termed inflammaging, although SASP may be only one of the possible causes of this condition. Chronic inflammation due to SASP can suppress immune system function, which is one reason elderly persons are more vulnerable to COVID-19.

SASP induces an UPR in the cell because of an accumulation of unfolded proteins, resulting in proteotoxic impairment of cell function (Saez-Atienzar and Masliah 2020.

Ablation of senescent cells (senolytic therapy) has been postulated as a promising therapeutic approach to target the ageing phenotype and, thus, to prevent, delay or mitigate ageing-related, and many other diseases. The aim with senolytic compounds is to rejuvenate organisms by selectively killing senescent cells and their efficacy is based on the ability of senescent cells to resist apoptosis. These cells exhibit upregulation of pro-survival pathways to protect themselves from the damaging (and pro-apoptotic) effect of the SASP. To date, six pro-survival pathways have been detected in senescent cells: the BCL-2-BCL-xL pathway, the MDM2-p53-p21Cip1-serpine elements pathway, ephrins-dependence receptors-tyrosine kinases, the PI3K-AKT-ceramide metabolic pathway, the hypoxia inducible factor 1α (HIF-1α) pathway and the SP-90-dependent pathway. Senolytic compounds interfere with these pro-survival pathways to let senescent cells die by apoptosis (Saez-Atienzar and Masliah 2020.

Clearing already formed senescent cells, many of which harbour oncogenic mutations or cause cancer-promoting inflammation due to their SASP, prevents or delays cancer development. Indeed, senolytic drugs, agents that selectively eliminate senescent cells, delay development of multiple forms of cancer in old mice as well as cancer-prone, DNA repair-deficient Ercc1-/D mice.

Furthermore, although capacity for cells to become senescent is a cancer defence mechanism, already formed, chronically persisting senescent cells can accelerate cancer growth because of suppression of immune responses, growth factors in the SASP, production of proteases that can interfere with encapsulation of tumours and therefore can enhance spread of cancer, and promotion of mesenchymal transition of cells. Thus, targeting transcription factor cascades that allow cells to become senescent can exacerbate cancer development, whilst clearing already formed senescent cells with senolytics can prevent cancer development, growth and spread (Saez-Atienzar and Masliah 2020; Thoppil and Riabowol 2020).

Therefore p53, p21, pRb, BCL-2, AKT, SP-90 and others involved in these pathways are target of the Senolytic compounds (Saez-Atienzar and Masliah 2020; Thoppil and Riabowol 2020).

The present invention demonstrate that NMDA receptor antagonists increase the proteasome activity and cause a decrease in p21, p53, pAkt1 and ppRb which are the major target molecules of the senolytic drugs (FIGS. 1, 2A and 34).

According to some embodiments, the present invention provides, in one aspect, a pharmaceutical composition comprising at least one NMDA receptor antagonist or a pharmaceutically acceptable salt, hydrate or pharmaceutically active enantiomer thereof, for use in ablation of senescent cells which has been postulated as a promising therapeutic approach to target the ageing phenotype and, thus, to prevent, delay or mitigate ageing-related diseases

Therefore, According to an aspect of some embodiments of the present invention there is provided an NMDA receptor antagonist for use in treating ageing which is caused by cellular senescence and associated with increasing risk for developing multiple chronic diseases, the geriatric syndromes, impaired physical resilience and mortality.

Amongst the diseases with emerging evidence for a causal contribution of cellular senescence or benefits of senolytics include, but are not limited to, Diabetes/Obesity, metabolic diseases, Cardiac dysfunction, congestive heart failure, myocardial infarction, Vascular hyporeactivity/calcification, AV fistulae, Frailty, Age-related muscle loss (Sarcopenia), arthritis, osteoporosis, falls, Chemotherapy complications, Radiation complications, Cancers, Bone marrow transplant complications, Organ transplantation complications, Myeloma/MGUS (monoclonal gammopathy of undetermined significance), Age-related cognitive dysfunction, other dementias, Alzheimer's disease, Parkinson's disease, Amyotrophic lateral sclerosis, Ataxia, Obesity-related neuropsychiatric dysfunction, Renal dysfunction, Urinary incontinence, Osteoporosis, Osteoarthritis, Age-related intervertebral disc disease, Idiopathic pulmonary fibrosis, Hyperoxic lung damage, Chronic obstructive pulmonary disease, Tobacco, Hepatic steatosis, Cirrhosis, Primary biliary cirrhosis, Progerias, Pre-eclampsia, Macular degeneration, Glaucoma, Cataracts, blindness, Prostatic hypertrophy, incontinence, Psoriasis, Healthspan, Lifespan and many others. (Kirkland and Tchkonia, 2020)

The 20S proteasome plays a pivotal role in the selective recognition and degradation of oxidized proteins, being responsible for the degradation of approximately 90% of all intracellular oxidation-damaged proteins. Notably, degradation by the 20S proteasome occurs in a Ub-independent manner. Studies indicate that the cell is able to reversibly disassemble 26S proteasomes to elevate the levels of free 20S particles under oxidative conditions or in response to mitochondrial dysfunction (Ishiie et al. 2005).

The accumulation of unfolded, misfolded, or damaged proteins severely impairs the function of organelles. Several reports indicate that one of the first detectable consequences of proteasome dysfunction concerns mitochondria, which develop deleterious alterations in their proteome. Mitochondria are the cellular power plants that produce energy through oxidative phosphorylation. They also directly contact most (if not all) other cytosolic organelles (e.g., ER, plasma membrane, and peroxisomes) to generate specialized networks that control several cellular functions like Ca2+ homeostasis, lipid synthesis, and apoptosis. Importantly, dysfunctional mitochondria are also the major source for oxidative stress through aberrant production of ROS, which has profound implications for the pathogenesis of various diseases, in particular neurodegenerative disorders and cancer, as well as aging (Pan et al. 2021).

The mitochondria are involved in a range of other processes, such as signaling, cellular differentiation, cell death, as well as the control of the cell cycle and cell growth. Given their entral role in cell metabolism, damage and subsequent dysfunction in mitochondria is an important factor in a wide range of human diseases and may play role in the aging process.

The protesome pathway preserve mitochondrial plasticity and quality by controlling several levels of mitochondrial dynamics. These activities and processes are tightly interconnected. During moderate stress, as a first line of defense against mitochondrial dysfunction, the UPS is in charge of outer membrane PQC and the degradation of damaged proteins in a process called outer mitochondrial membrane-associated degradation (OMMAD). Moreover, the UPS directly controls mitochondrial dynamics by degrading proteins involved in mitochondrial fusion and fission processes. While the 20S proteasome has largely been considered a dormant protease that is only activated upon binding to regulatory complexes, significant and increasing evidence has clearly demonstrated that the 20S core has a major a role in overall protein degradation, independent of ubiquitin and ATP (Raynes et al. 2016).

Therefor the present invention can provide effective treatment for, reduction of symptoms and even prophylaxis of, certain mitochondrial diseases, directly or indirectly, including, Mitochondrial myopathy, Diabetes mellitus and deafness, Leber's hereditary optic neuropathy, Leigh syndrome, subacute sclerosing encephalopathy, Neuropathy, ataxia, retinitis pigmentosa, ptosis, progressive symptoms of dementia, Myoneurogenic gastrointestinal encephalopathy, MERRF syndrome (Myoclonus Epilepsy with Ragged Red Fibers), MELAS syndrome (Mitochondrial Encephalopathy, Lactic Acidosis and Stroke-like episodes), Mitochondrial DNA depletion syndrome, Huntington's disease, cancer, Alzheimer's disease, Parkinson's disease, bipolar disorder, schizophrenia, aging and senescence, anxiety disorders, Alper syndrome, Lowe syndrome, Luft syndrome, Menke's kinky hair syndrome, Zellweger syndrome, mitochondrial myopathy, and rhizomelic chondrodysplasia punctata.

Therefore, the present invention further provides, in another aspect, a method for correcting and/or treating the impaired autophagy and mitophagy and related diseases mentioned above.

Endoplasmic reticulum is an organelle that performs various functions such as lipid and protein synthesis, protein folding, calcium homeostasis. In this organelle, an increase and accumulation in unfolded or misfolded proteins disrupts functions of the organelle and causes endoplasmic reticulum stress. The complex signalling pathways triggered by ER stress constitute the UPR. Endoplasmic reticulum stress is seen in neurodegenerative diseases, metabolic diseases, arteriosclerosis, diabetes mellitus and obesity. Endoplasmic reticulum stress is also associated with cancer (Ding et al. 2007; Roussel 2013).

The present invention provides, in one aspect, a pharmaceutical composition comprising at least one NMDA receptor antagonist or a pharmaceutically acceptable salt, hydrate or pharmaceutically active enantiomer thereof, for use in treating a the endoplasmic reticulum (ER) related diseases, directly or indirectly, including by decreasing ER stress. Metabolic diseases including, Insulin resistance, Arteriosclerosis, Diabetes mellitus, Obezite, alcoholic and non-alcoholic fatty liver disease, hyperlipidemia; cancers including Leukemia, multiple myeloma, breast cancer, prostate tumor; immune system related diseases including, viral infections, Bacterial infections, Vitiligo, rheumatoid arthritis, and type 1 diabetes. Many neurological diseases are known to be associated with ER stress, including cerebral ischaemia, sleep apnea, Alzheimer's disease, multiple sclerosis, amyotrophic lateral sclerosis, the prion diseases, Parkinson's and Huntington's diseases and familial encephalopathy with neuroserpin inclusion bodies.

Autophagy lysosome pathway is another degradation pathway in which cytoplasmic components, such as protein aggregates and damaged organelles are degraded and recycled for maintaining normal cellular homeostasis. In this pathway cytoplasmic materials are delivered to lysosomes, sequestered into autophagosome, which subsequently fuse with lysosomes, where the cargo is degraded.

Depending on the context, autophagy and the proteasome share common substrates as well as regulatory factors. Both systems intersect and communicate at multiple points to coordinate and balance their actions in proteostasis and homeostasis of organelles (Liu et al. 2020; Ding et al. 2007; Feldman et al. 2019).

The most important concern that may occur as consequence of decreased protesome activity is the accumulation of potentially toxic un/misfolded proteins as well as protein aggregates. The proteasome impairment will eventually affect the vital function of autophagy and cytosolic processes and all other organelles due to the accumulation of unfolded and damaged proteins (Dikic 2017; Pohl and Dikic 2019, Kocaturk and Gozuacik, 2018).

The present invention encompasses the finding that activation of proteasome by NMDA inhibitors, through increased levels and/or increased activity, can provide effective treatment for, and even prophylaxis of, certain autophagy related human diseases, such as adult neurodegenerative disorders including Parkinson's disease, Amyotrophic lateral schlerosis, Frontotemporal dementia, Neuronal ceroid lipofuscinosis, Fulminant neurodegeneration, Dementia with Lewy bodies; Pediatric Neurodevelopmental disorders including Spinocerebellar ataxia, Cortical atrophy and epilepsy, Childhood-onset neurodegeneration, BPAN, Spastic quadriplegia and brain abnormalities, Primary microcephaly, Hereditary spastic paraplegia, Ataxia with spasticity, Rett syndrome, Joubert syndrome, Leukoencephalopathy, Adolescent-onset dystonia, CEDNIK syndrome, Pelizaeus-Merzbacher-like disorder, West syndrome; Hereditary neuropathies including Sensory and autonomic neuropathy type II, Charcot-Marie-Tooth disease, Sensory and autonomic neuropathy type IF, Distal hereditary motor neuronopathy; Ophthalmological diseases including Primary open-angle glaucoma, Cataracts; Cardiac and skeletal myopathies including Danon's cardiomyopathy, Distal myopathy with rimmed vacuole, Dilated cardiomyopathy, Sporadic inclusion body myositis, X-linked myopathy with excessive autophagy; Inflammatory disorders including Crohn's disease, Ulcerative colitis, Childhood asthma; Autoimmune diseases including Systemic lupus erythematous, Diabetes, Other autoimmune diseases; Infectious diseases including M. tuberculosis, M. leprae; Skeletal disorders including Osteopetrosis, Paget's disease of the bone, Kashin-Beck disease; Congenital multisystem disorders including Global developmental abnormalities, Vici's syndrome, Zellweger's syndrome, Glycosylation disorder with autophagy defects, Zimmerman-Laband syndrome, Hermansky-Pudlak syndrome, Multisystem proteinopathy.

The present invention encompasses the finding that activation of proteasome by NMDA inhibitors, prevents the malfunctioning of the protein homeostasis network including accumulation of oxidatively damaged and/or misfolded proteins which are associated with lysosome dysfunctions. The present invention encompasses the finding that activation of proteasome by NMDA inhibitors, through increased levels and/or increased activity, can provide effective treatment for, and even prophylaxis of, certain Lysosomal Storage Diseases. Lysosomal storage diseases include Gaucher disease, Fabry disease, Niemann-Pick disease, Hunter syndrome, Glycogen storage disease II (Pompe disease), Tay-Sachs disease.

Importantly, in any case, attempts to improve proteostasis pharmacologically have to be at an early stage of disease before the manifestation of severe cellular dysfunction. Indeed, present invention demonstrated that starting treatment early is more effective in increasing life expectancy than starting late (Salomon et al. 2012; Halminen et al. 2021).

Glutamate receptors key component of the glutamate pathway, exhibit their role as signal detectors and transmitters. Glutamate receptors are mainly subdivided into two groups; ionotropic glutamate receptors (iGluRs) that constitute ion channels, and metabotropic glutamate receptors (mGluRs) which are members of the G protein-coupled receptor (GPCR) superfamily.

NMDA receptors belong to the iGluRs family comprising Kainate receptors, (2-amino-3(3-hydroxy-5-methylisoxazol-4-yl)propanoic acid) AMPA and (N-methyl-D-aspartate receptor) NMDA receptors.

Unlike other iGluRs, NMDA receptors are well-known for their high Ca2+ ion conductance, voltage dependent-channel blockade, and the compulsory binding of two endogenous ligands for receptor activation. Notwithstanding the tight regulation of glutamate synaptic concentration, excessive glutamatergic signaling is a key feature of neurodegenerative pathologies resulting in drastically elevated intracellular Ca2+ ions levels and subsequent neuronal apoptosis.

Structurally, NMDA receptors are heterotetrameric ion channels composed of four residues derived from three related subunits: GluN1 subunit, GluN2 subunit and GluN3 subunit. The activation of NMDA receptors relies on the combination of two agonists, that are glutamate and glycine or D-serine. Usually, NMDA receptors consist of two glycine binding GluN1 subunits and two glutamate-binding GluN2 subunits. Specifically, eight isoforms of GluN1 exist (a-h, different splice variants of one single gene), four GluN2 (A-D, encoded by four different genes) and two GluN3 (A-B, encoded by two distinct genes) subunits have been identified. There are multiple ligand-binding sites on the NMDA receptors including glutamate binding sites, glycine binding sites, ion channels pore, and allosteric binding sites on the aminoterminal domain (ATD), which modulate receptor activity in a subtype-selective manner. More and more studies have shown that different subtypes of NMDA receptors generate different functional outputs. Most human-specific GluN splice variants occur within the amino terminal domain (ATD) and CTD. Corresponding sequences may easily be obtained in current databases, like the EMBL-EBI data base under www.ebi.ac.uk or the NCBI database under www.incbi.nln.nih.gov (Cull-Candy et al. 2001; Ishchenko et al. 2021; Paoletti et al. 2013).

The term “NMDA receptor antagonist or blocker or inhibitor” as employed herein relates to substances which can negatively modulate NMDA receptors/NMDA complexes. Said “negative modulation” comprises an inhibition and/or blockage. Said inhibition and/or blockage may be partial or complete. The antagonist to be employed in context of this invention, most preferably, inhibits/blocks (an) human NMDA receptor(s).

In certain embodiments, NMDA receptor antagonist’ relates to compounds which are in vivo and/or in vitro capable to block, either completely or partially, the action and/or function of the NMDA receptor or the NMDA receptor complex.

In certain embodiments, the NMDA receptor antagonist preferentially binds to extra-synaptic NMDA receptors. In certain embodiments, the extra-synaptic NMDA receptors comprise a NR2B subunit.

As of today, three major classes of NMDAR antagonists can be distinguished based on their mechanism of action: competitive antagonists, uncompetitive channel blockers, and non-competitive allosteric inhibitors. Although ultimately resulting in a similar outcome, each antagonist class has a distinct mechanism of action. Competitive NMDAR antagonists act by occupying the glycine or glutamate-binding site located on either the GluN1 or GluN2 subunit, respectively. This class of antagonist prevents endogenous ligand binding, thereby preventing activation of the receptor. Competitive and uncompetitive antagonists tend to be poorly selective for a specific GluN1/GluN2 subunit, whereas noncompetitive allosteric inhibitors display much higher selectivity. Uncompetitive channel blockers act by occluding the NMDAR ion channel pore.

The class of uncompetitive ion channel blockers are ketamine, dizocilpine (MK-801), and memantine (Petit-Pedrol et al. 2021; Das 2020; Siala et al 2020).

Uncompetitive channel blockers of NMDAR are typically positively charged and require depolarization, such that the Mg2+block is removed and the binding site exposed. Once bound, these channel blockers can prevent the flow of ions in/out of the channel thus producing inhibition. It is important to note that when channel blockers are in the pore, glutamate may still bound to the NMDAR potentially resulting in Ca2+-independent intracellular signaling; though the significance of this phenomena is not fully understood. Finally, non-competitive allosteric modulators inhibit activity of NMDARs by acting at the N-terminal or “agonist binding” domains of the GluN1/GluN2 subunits to reduce the activity of the receptor by decreased binding of endogenous ligands. non-competitive NMDAR allosteric inhibitors act at extracellular domains (e.g. Zn2+, NO), and NMDAR channel blockers block the open channel following activation by the agonists.

All competitive antagonists discriminate poorly between the different NMDAR subtypes NR1/NR2(A-D) and therefore cause generalized inhibition of NMDARs (Liu et al. 2020; Vieira et al 2020; Groc et al. 2009).

NMDA receptors (NMDARs) are essential for normal physiological processes in the central nervous system, e.g. development, induction of synaptic plasticity, learning and memory. It was demonstrated that specific glutamate receptors and glutamate transporters were also expressed in non-neurological tissues, such as the lung, liver, kidney, stomach and immune system, which suggested that NMDARs play an important role in the regulation of physiological function in various peripheral organs (Du etall. 2016).

According to some embodiments, the NMDA antagonist is a specific inhibitor of glutamatergic neurotransmission. According to some embodiments, the NMDA antagonist may be a less specific antagonist that acts on other neurotransmission pathways. According to some embodiments, the NMDA antagonist may have additional activities such as serotonergic, cholinergic or dopaminergic antagonist or agonist activities.

The channel blocker memantine was approved for the treatment of moderate-to-severe Alzheimer's disease. Memantine's unusual clinical tolerance may well reflect its low affinity binding to open channels and its relatively fast unblocking kinetics (Petit-Pedrol et al. 2021; Das 2020; Siala et al 2020).

Blockers remaining trapped in the pore during agonist unbinding, like ketamine or (−)MK-801, showed stronger dependence on extracellular pH than others, like (+)MK-801, memantine or dextromethorphan. Acidic extracellular pH increased the association rate of (−)MK-801 with the intrapore binding site of the NMDAR, which appears to be the underlying mechanism for pH-dependent potency boost.

Another salient observation is that the potency of channel block of a structurally diverse group of compounds varies for NMDARs with different NR2 subunits, even at physiological pH. The >10-fold higher potency of (−)MK-801 and (+)ketamine for NR1/NR2B versus NR1/NR2A receptors could be the basis for the development of new truly subunit-selective NMDAR channel blockers. Clinically promising subunit-selective NMDAR channel blockers should show in addition pH dependence and, similar to memantine, fast channel unblocking kinetics to prevent the drug from occupying the channels and interfering with normal synaptic transmission. Memantine is therefore very different from (+)MK-801, which binds with higher affinity and has relatively slower unblocking kinetics. Because of these properties (+)MK-801 has been used for the last 20 years as a pharmacological tool to irreversibly block NMDARs but has failed in clinical trials (Petit-Pedrol et al. 2021; Das 2020; Siala et al 2020).

Non-limiting examples of NMDA receptor antagonists include, memantine (3,5-dimethyladamantan-1-amine), nitromemantine (3-amino-5,7-diethyladamantan-1-yl nitrate), Fluoroethylnormemantine (FENM or FNM), ketamine (2-(2-chlorophenyl)-2-(methylamino)-cyclohexanone)), Esketamine ((2S) 2-(2-chlorophenyl)-2-(methylamino)-cyclohexanone)), (+)-norketamine ((+)-2-Amino-2-(2-chlorophenyl) cyclohexanone hydrochloride), (S)-ketamine, (R,S)-ketamine, dextromethorphan (DXM), Amitriptyline (N,N-dimethyl-3-(2-tricyclo [9.4.0.03,8]pentadeca-1(15),3,5,7,11,13-hexaenylidene)propan-1-amine), Ifenprodil (α-(4-Hydroxyphenyl)-3-methyl-4-benzyl-1 piperidineethanol(+)-tartrate salt), Orphenadrine (β-Dimethylaminoethyl2-methylbenzhydryl ether citrate salt), TCN-201 (3-Chloro-4-fluoro-N-[(4-{[2 (phenylcarbonyl) hydrazino]carbonyl}phenyl) methyl]benzenesulfonamide), SDZ 220-581 ((S)-a-Amino-2-chloro-5-(phosphonomethyl) [1,1?bipbenyl]-3-propanoic acid hydrochloride), 5,7 Dichlorokynurenic Acid (5,7-Dichloro-4-hydroxyquinoline-2-carboxylic acid monohydrate), MD-Ada (1-N-Methylamino-3,5-dimethyl adamantane hydrochloride), AP-7 (DL-2-Amino-7-phosphonoheptanoic acid), Ro8-4304 (4-{3-[4-(4-fluorophenyl)-3,6-dihydro-2H pyridin-1-yl)-2hydroxy-propoxy)-benzamide), L-687,384 (1′-benzylspiro[2,3-dihydro-1H-naphthalene-4,4′-piperidine]), Spermine (N,N′-Bis(3-aminopropyl)-1,4-diaminobutane), DCOX (6,7-Dichloro-2, 3 quinoxalinedione), Traxoprodil ((18,2S)-1-(4-hydroxy-phenyl)-2-(4-hydroxy-4phenylpiperidino)-1-propanol), Fanapanel ([[3,4-Dihydro-7-(4-morpholinyl)-2,3-dioxo-6 (trifluoromethyl)-1(2H)-quinoxalinyl]methyl]-phosphonic acid hydrate), Metaphit (1-[1-(3-Isothiocyanato) phenyl]cyclobexylpiperidine methanesulfonate salt), NAAG (N Acetylaspartyl glutamic acid, 5-Fluoroindole-2-carboxylic acid, (S)-(−)-4-Oxo-2-azetidinecarboxylic acid, Benzyl(S)-(−)-4-oxo-2-azetidinecarboxylate, (+)-a-Amino-3-carbomethoxy-5-methylisoxazole-4 propanoic acid), CP-101,606 (traxoprodil)(1S,2S)-1-(4-hydroxy-pbenyl)-2-(4-hydroxy-4-pbenylpiperidino)-1-propanol), Neu 2000 (5-Aminosalicylic acid), TCS 46b (1,3-Dihydro-5-[3-[4-(phenylmethyl)-1-2H-benzimidazol-2-one), O-Phospho-L-serine (L-SOP) (L-2-Amino-3-hydroxypropanoic acid 3-phosphate), (+/−)-Huperzine A ((2S)-2-Amino-4-phosphonobutanoic acid), Gavestinel (1H-Indole-2-carboxylic acid, 4,6-dichloro-3-[(1E)-3-oxo-3-(pbenylamino)-1-propen-1-yl]-3-(3-anilino-3-oxoprop-1-enyl)-4,6-dichloro-1H-indole-2-carboxylic acid), gamma-dgg (D-γ-Glu-Gly), N20C ydrochloride (N2-(3,3-Diphenylpropyl) glycinamide hydrochloride (1:1)), (1)-CPP ((1)-3-(2-Carboxypiperazin-4-yl) propyl-1-phosphonic acid), histogranin, meperidine, methadone, methoxetamine (MXE), phencyclidine (PCP) (1-(1-Phenylcyclohexyl)piperidine), nitrous oxide (N20), D(−)-AP-5 (D(−)-2-Amino-5-phosphonopentanoic acid), AP7 (2-amino-7-phosphonoheptanoic acid), CPPene ((3-[(R)-2-carboxypiperazin-4-yl]-prop-2-enyl-phosphonic acid), AP-5 (DL-2-Amino-5-phosphonovaleric acid), Selfotel, Amantadine (adamantan-1-amine), Atomoxetine, AZD6765 (as-a-Phenyl-2-pyridineethanamine dihydrochloride), Agmatine, chloroform, EAA-090 ((P-[2-(8,9-dioxo-2,6 diazabicyclo[5.2.0]non-1(7)-en-2yl) ethyl]-phosphonic acid), D-AP-7 (D(−)-2-Amino-7-phosphono heptanoic acid), dextrallorphan, dextromethorphan, dextrorphan ((+)-3-Hydroxy-N-methylmorphinan(+) -tartrate salt), diphenidine, dizocilpine (MK-801) ((5S,10R)-(+)-5-Methyl-10,11-dihydro-5H dibenzo [a,d]cyclobepten-5,10-imine maleate), ethanol, eticyclidine, gacyclidine, ibogaine, magnesium, rolicyclidine, Felbamate (2-Phenyl-1,3-propanediol dicarbamate),
tenocyclidine, methoxydine, tiletamine, neramexane (1,3,3,5,5-pentamethyl cyclohexan-1-amine), eliprodil (a-(4-Chlorophenyl)-4-[(4-fluorophenyl) methyl]-1 piperidineethanol), dexoxadrol, etoxadrol, Ro 25-6981 ([R-(R*,S*)]-a-(4-Hydroxyphenyl)-b-methyl-4(phenylmethyl)-1-piperidinepropanol hydrochloride hydrate), MDL 105,519 ((E)-4,6-Dichloro-3-(2-phenyl-2-carboxyethenyl)indole-2carboxylic acid), remacemide, delucemine, WMS-2539, NEFA, 8A-PDHQ, HU-211, Aptiganel (Cerestat, CNS-1102), rhynchophylline, kynurenic acid (4-Hydroxyquinoline-2-carboxylic acid), Rapastinel (GLYX-13), NRX-1074, 7-Chlorokynurenic acid, 4-Chlorokynurenine (AV-101), TK-40, 1-Aminocyclo propanecarboxylic acid (ACPC), L-Phenylalanine, Xenon, [Glu3,4,7,10,14]
Conantokin G (Gly-Glu-Glu-Glu-Leu-Gln-Glu-Asn-Gln-Glu-Leu-Ile-Arg
Glu-Lys-Ser-Asn), 1-amino-alkylcyclohexanes, licostinel (6,7-dichloro-5-nitro-1,4-dihydro-2,3-quiNoxalinedione), ACEA 1021, CP-101606, EAB-318 (R-alpha amino-5-chloro-1-(phosphonomethyl)-1H-benzimidazole 2-propanoic acid hydrochloride); CP-101606, Ro-25-6981, CoI01244, L-701,324, CGP401 16, LY235959, LY233053, MRZ2/576, LU73068,4-Cl-KYN and analogs or derivatives, pharmaceutically acceptable salts, hydrates and pharmaceutically active enantiomers thereof. Ketamine derivatives such as Rapastinel or Glyx-13 are also included.

Non-limiting examples of the NMDA receptor antagonists also include anti-receptor antibodies, anti-ligand antibodies, inhibitory nucleic acids, etc.

Several synthetic opioids function as NMDA receptor-antagonists, such as pethidine, methadone, meperidine, dextropropoxyphene, tramadol, levorphanol, and ketobemidone.

Each possibility represents a separate embodiment of the invention.

In certain embodiments, the NMDA receptor antagonist is selected from the group consisting of memantine, nitromemantine, FENM, neramexane, ketamine, amantadine, dextromethorphan, L-687,384, amitriptyline, 1-benzyl-6 methoxy-6′,7′-dihydrospiro-[piperidine-4,4-thieno [3.2-c]pyran), eliprodil, ifenprodil, orphenadrine, felbamate, and pharmaceutically acceptable salts, hydrates and pharmaceutically active enantiomers thereof. Each possibility represents a separate embodiment of the invention.

In certain embodiments, the NMDA receptor antagonist is selected from the group consisting of memantine, nitromemantine, neramexane, ketamine and pharmaceutically acceptable salts, hydrates and pharmaceutically active enantiomers thereof. Each possibility represents a separate embodiment of the invention.

According to some embodiments of the invention, the NMDA receptor antagonist is selected from the group consisting of memantine, eliprodil and ifenprodil or a pharmaceutically acceptable salt, hydrate or pharmaceutically active enantiomer thereof.

In certain embodiments, the NMDA receptor antagonist is memantine (3,5-dimethyl-1-adamantanamine hydrochloride) or a pharmaceutically acceptable salt, hydrate or pharmaceutically active enantiomer thereof.

Memantine is described in detail herein and further details may be found in U.S. Pat. No. US 2017/0273917 A1; U.S. Pat. No. 4,122,193; 4,273,774 or 5,061,703. Again, as pointed out herein above all the compounds used in accordance with this invention may also be employed in form of pharmaceutically acceptable salts, like memantine hydrochloride and the like.

Memantine is the first representative of a new class of Alzheimer drugs—a moderate affinity NMDA-receptor antagonist. Memantine has been developed by Merz Pharmaceuticals and was approved in Europe and the USA for the treatment of moderate to severe Alzheimer's disease.

According to specific embodiments, the NMDA receptor antagonist binds specifically the NMDA receptor with no cross reactivity with other receptors.

Several substances are selective for the NR2B NMDA receptor subtypes. The NMDAR2B subunit is the focus of increasing interest as a therapeutic target in a wide range of CNS pathologies, including acute and chronic pain, stroke and head trauma, drug-induced dyskinesias, and dementias. The apparent Superior preclinical and clinical data is likely to reflect Subtype selectivity, a unique mode of action and cellular location of the NR2B receptors in the CNS (Pender et al. 2020; Vieira et al. 2020). The NMDA NR2B subunit receptor specificant agonist, CP-101606, dose-dependently improved the rate of functional recovery and protected against the ischemic brain damage. It has been concluded that NMDA NR2B receptor Subunits represent potential targets to reduce not only the functional deficits, but also neuronal death in cortex and several midbrain regions produced by cerebral ischemia (Liu et al. 2020). Also eliprodil antagonises the NR2B subunit. Eliprodil has been shown to protect from NMDA receptor-mediated excitotoxicity during ethanol withdrawal. Col 01244, another novel potent and selective NR1/2B NMDA receptor antagonist, has been found promising in antiepileptic medication (Ahmed et al. 2020; U.S. Pat. Nos US 2017/0273917 A1).

Many compounds acting as NMDA receptor antagonists are known to possess further biological activities. Thus, according to other specific embodiments, the NMDA receptor antagonist can modulate the activity of other receptors.

As used herein, the term “modulate” refers to altering activity either by inhibiting (i.e. antagonist) or by activating (i.e. agonist) activity and/or expression of a receptor. According to specific embodiments, modulates activity and/or expression is inhibits activity and/or expression.

According to specific embodiments, modulates activity and/or expression is activates activity and/or expression. Thus, in certain embodiments, the NMDA receptor antagonist also has serotonergic activity. The term “serotonergic activity” as used herein refers to the ability of the NMDA receptor antagonist to also modulate the activity of a receptor of the 5-HT receptor family. As used herein, the term “5-HT receptor family” refers to a group of proteins that function as receptors for serotonin. The group contains subset of proteins which are encoded by genes which exhibit homology of greater than 72% or higher with each other in their deduced amino acid sequences within presumed transmembrane regions (linearly contiguous stretches of hydrophobic amino acids, bordered by charged or polar amino acids, that are long enough to form secondary protein structures that span a lipid bilayer). Four human 5-HT receptor subfamilies are known to date: 5-HT1, 5-HT2, 5-HT3, and 5-HT4.

In certain embodiments, the NMDA receptor antagonist is also a 5-HT2 receptor antagonist.

In certain embodiments, the NMDA receptor antagonist having 5-HT2 receptor antagonist activity is ketamine, or pharmaceutically acceptable salts, hydrates or pharmaceutically active enantiomers thereof. Each possibility represents a separate embodiment of the invention.

In certain embodiments, the NMDA receptor antagonist also has cholinergic activity. The term “cholinergic activity” as used herein refers to the ability of the NMDA receptor antagonist to also modulate the activity of a receptor of the nicotinic acetylcholine receptor (nACHR) family. In certain embodiments, the NMDA receptor antagonist is also a nicotinic acetylcholine receptor (nACHR) antagonist.

As used herein, the term “nicotinic acetylcholine receptor (nACHR) family” refers to a group of proteins that function as receptors for acetylcholine that signal for muscular contractions upon a ligand binding. In certain embodiments, the NMDA receptor antagonist having nACHR antagonist activity is selected from the group consisting of amantadine and dextromethorphan, and pharmaceutically acceptable salts, hydrates and pharmaceutically active enantiomers thereof. Each possibility represents a separate embodiment of the invention.

In certain embodiments, the NMDA receptor antagonist also has dopaminergic activity. The term “dopaminergic activity” as used herein refers to the ability of the NMDA receptor antagonist to also modulate the activity of a receptor of the dopamine receptor family.

As used herein, the term “dopamine receptor family” refers to a group of proteins that function as receptors for dopamine. The group contains subset of proteins which are encoded by genes which exhibit homology of greater than 65% with each other in their deduced amino acid sequences within presumed transmembrane regions. Three human dopamine receptor subfamilies are known to date: dopamine D receptor, dopamine D2 receptor and dopamine D3 receptor.

In certain embodiments, the NMDA receptor antagonist is also a dopamine D2 receptor agonist.

In certain embodiments, the NMDA receptor antagonist having dopamine D, receptor agonist activity is ketamine, or pharmaceutically acceptable salts, hydrates or pharmaceutically active enantiomers thereof. Each possibility represents a separate embodiment of the invention.

In certain embodiments, the NMDA receptor antagonist having sigma-1 receptor agonist activity is selected from the group consisting of L-687, 384, amitriptyline and 1-benzyl-6-methoxy-6,7′-dihydrospiro?piperidine-4,4′-thieno [3.2-c]pyran], and pharmaceutically acceptable salts, hydrates and pharmaceutically active enantiomers thereof. Each possibility represents a separate embodiment of the invention.

As used herein, the term “sigma-1 receptor” refers to an expression product of the SIGMAR1 gene which encodes a chaperone protein at the endoplasmic reticulum (ER) that modulates calcium signaling through the IP3 receptor.

A subject in need of such treatment or prevention a therapeutically effective amount of lower than 10 mg/kg of an NMDA receptor antagonist, memantine.

In certain embodiments, the effective amount of the present agent is a dose of about 0.01 to about 10 mg per kilogram of body weight of the subject (mg/kg), i.e., from about 0.01 mg/kg to about 10 mg/kg body weight. In certain embodiments, the effective amount of the present compound ranges 0.001 to approximately 9 mg/kg body weight, 0.001 to approximately 8 mg/kg body weight, from about 0.01 mg/kg to about 7 mg/kg body weight, from about 0.01 to about 2 mg/kg of body weight, about 0.01 to about 6 mg/kg of body weight, about 0.05 to about 5 mg/kg of body weight, about 0.05 to about 4 mg/kg of body weight, about 0.05 to about 3 mg/kg of body weight, about 0.05 to about 2 mg/kg of body weight, about 0.01 to about 1.5 mg/kg of body weight, about 0.01 to about 1.3 mg/kg of body weight, about 0.01 to about 1.2 mg/kg of body weight, about 0.01 to about 1.1 mg/kg of body weight, about 0.01 to about 1.0 mg/kg of body weight, about 0.01 to about 0.9 mg/kg of body weight, or about 0.05 to about 0.8 mg/kg of body weight. In some aspects, the dose is about 0.05 to about 0.5 mg/kg. In some aspects, the dose is less than about 0.5 mg/kg, less that about 0.4 mg/kg, or less than about 0.3 mg/kg body weight. In some aspects, the effective amount of the present compound is a dose in the range of from about 0.01 mg/kg to about 1.5 mg/kg body weight. In some aspects, the effective amount of the present compound is a dose in the range of from about 0.01 mg/kg to about 1 mg/kg body weight. In some aspects, the effective amount of the present compound is a dose in the range of from about 0.01 mg/kg to about 0.75 mg/kg body weight. In some aspects, the effective amount of the present compound is a dose in the range of from about 0.75 mg/kg to about 1.5 mg/kg body weight. In some aspects, the effective amount of the present compound is a dose in the range of from about 0.5 mg/kg to about 1.2 mg/kg body weight. In some aspects, the effective amount of the present compound is a dose in the range of from about 0.05 mg/kg to about 0.5 mg/kg. In some aspects, the effective amount of the present compound is a dose of about 0.2 mg/kg or about 0.4 mg/kg body weight. In some aspects, the dose of the present compound is, about 0.01 to about 1 mg/kg, about 0.1 to about 0.5 mg/kg, about 0.8 to about 1.2 mg/kg, about 0.7 to about 1.1 mg/kg, about 0.05 to about 0.7 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, or about 3 mg/kg body weight.

In certain embodiments, the dose of the present compound per administration is from about 1 to about 250 mg, from about 10 mg to about 300 mg, about 10 mg to about 250 mg, about 10 to about 200 mg, about 15 to about 175 mg, about 20 to about 175 mg, about 8 mg to about 32 mg, about 50 mg to about 75 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg, about 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, and 250 mg. In some aspects, the dose of the present compound is about 50 mg. In some aspects, the dose of the present compound is about 75 mg. In some aspects, the total dose of the present compound is about 100 mg.

In certain embodiments, a therapeutically effective dose of the NMDAR blockers may be adjusted depending on conditions of the disease/disorder to be treated or prophetically treated, the age, body weight, general health conditions, sex, and diet of the subject, dose intervals, administration routes, excretion rate, and combinations of drugs. An initial dose of the present agent may be larger, followed by one or more smaller maintenance doses. Other ranges are possible, depending on the subject's response to the treatment. An initial dose may be the same as, or lower or higher than subsequently administered doses.

The present agent/composition may be administered daily, weekly, biweekly, several times daily.

The duration and frequency of treatment may depend upon the subject's response to treatment.

In certain embodiments, a subject may be administered 1 dose, 2 doses, 3 doses, 4 doses, 5 doses, 6 doses or more of the present agent/composition.

The number and frequency of doses may be determined based on the subject's response to administration of the composition, e.g., if one or more of the patient's symptoms improve and/or if the subject tolerates administration of the composition without adverse reaction.

In certain embodiments, the present agent/composition is administered at least once a day, at least twice a day, at least three times per day, or more.

In certain embodiments, the present agent/composition is administered to a subject prior to stressor. Wherein it is the accumulation of potentially toxic un/misfolded proteins as well as protein aggregates referred to as stressor. In addition, the proteasome impairment also referred to as stressor dependent on the accumulation of potentially toxic un/misfolded proteins as well as protein aggregates or independent. Therefore in certain embodiments, the present agent/composition is administered to the subject which is a person 25-year-old, 26-year-old, 27-year-old, 28-year-old, 29-year-old, 30-year-old, 31-year-old, 32-year-old, 33-year-old, 34-year-old, 35-year-old, 36-year-old, 37-year-old, 38-year-old, 39-year-old, 40-year-old, 41-year-old, 42-year-old, 43-year-old, 44-year-old, 45-year-old, 46-year-old, 47-year-old, 48-year-old, 49-year-old, 50-year-old, 51-year-old, 52-year-old, 53-year-old, 54-year-old, 55-year-old, 56-year-old, 57-year-old, 58-year-old, 59-year-old, 60-year-old, 61-year-old, 62-year-old, 63-year-old, 64-year-old, 65-year-old, 66-year-old, 67-year-old, 68-year-old, 69-year-old, 70-year-old, 71-year-old, 72-year-old, 73-year-old, 74-year-old, 75-year-old, 76-year-old, 77-year-old, 78-year-old, 79-year-old, 80-year-old, 81-year-old, 82-year-old, 83-year-old, 84-year-old, 85-year-old, 86-year-old, 87-year-old, 88-year-old, 89-year-old, 90-year-old, 91-year-old, 92-year-old, 93-year-old, 94-year-old, 95-year-old, 96-year-old, 97-year-old, 98-year-old, 99-year-old, 100-year-old, or older.

In certain embodiments, the present agent/composition is administered to the subject which is a person younger than 25-year-old.

In certain embodiments, the present agent/composition is administered to the subject throughout life.

A subject of this invention can be a mammal and in particular embodiments is a human, which can be an infant, a child, an adult or an elderly adult. The terms “subject” and “individual” are used interchangeably and relate to mammals. For example, mammals in the context of the present invention are humans, non-human primates, domesticated animals such as dogs, cats, sheep, cattle, goats, pigs, horses etc.

The present agent or composition may be administered to a subject alone, or may be administered to a subject in combination with one or more other treatments/agents.

In certain embodiments, combination therapy means simultaneous administration of the agents in the same dosage form, simultaneous administration in separate dosage forms, or separate administration of the agents.

In certain embodiments, the second agent/treatment is used as adjunctive therapy to the present agent or composition. In certain embodiments, the treatment includes a phase wherein treatment with the second agent/treatment takes place after treatment with the present agent or composition has ceased. In certain embodiments, the treatment includes a phase where treatment with the present agent or composition and treatment with the second agent/treatment overlap. Combination therapy can be sequential or can be administered simultaneously. In either case, these drugs and/or therapies are said to be “co-administered.”

In certain embodiments, a subject is treated concurrently (or concomitantly) with the present agent or composition and a second agent. In certain embodiments, a subject is treated initially with the present agent or composition, followed by cessation of the present compound or composition treatment and initiation of treatment with a second agent. In certain embodiments, the present agent or composition is used as an initial treatment, e.g., by administration of one, two or three doses, and a second agent is administered to prolong the effect of the present compound or composition, or alternatively, to boost the effect of the present agent or composition. A person of ordinary skill in the art will recognize that other variations of the presented schemes are possible, e.g., initiating treatment of a subject with the present agent or composition, followed by a period wherein the subject is treated with a second agent as adjunct therapy to the present agent or composition treatment, followed by cessation of the present agent or composition treatment. The present agent and the other pharmaceutically active agent(s) may be administered together or separately and, when administered separately this may occur simultaneously or sequentially in any order. The amounts of the present agent and the other pharmaceutically active agent(s) and the relative timings of administration will be selected in order to achieve the desired combined therapeutic effect.

In certain embodiments, a composition provided herein and a second agent are administered to a subject in a sequence and within a time interval such that the composition provided herein can act together with the other agent to provide an increased benefit than if they were administered otherwise.

The present invention provides that NMDA receptor antagonists activate the effects of proteasome 20S subunits including chemotrypsin, trypsin and caspase-like activities.

The term “activating agent”, as used herein, refers to an agent that increases level and/or activity of a target entity as compared with its level and/or activity under comparable conditions absent the activating agent. For example, an activating agent can increase level and/or activity of a target entity by at least about 5%, including at least about 10%, at least about 15%, at least about 20%, at least about 30%, at least about 40%, at least about 50%, at least about 60%, at least about 70%, at least about 80%, at least about 90%, at least about 95% or more, as compared with its level and/or activity under comparable conditions absent the activating agent. In some embodiments, an activating agent increases level and/or activity of its target entity to a point within a predetermined range of a reference level and/or activity. In some embodiments, a reference level and/or activity is the level and/or activity observed with a wild type version of the target entity in its natural context. In some embodiments, an activating agent binds directly to its target. In some embodiments, an activating agent binds indirectly (i.e., by binding with a physically distinct entity that binds to the target). In some embodiments, an activating agent does not interact physically, either directly or indirectly, with its target, but increases level and/or activity of the target through other action (e.g., binding to a regulatory site in a nucleic acid that increases expression of the target; activation or inhibition of an enzyme that modifies the target and alters its activity, etc). In some embodiments, an activating agent stabilizes and/or increases half-life of its target entity. In some embodiments, an activating agent stabilizes its target entity in a particular three-dimensional conformation. In some embodiments, an activating agent competes with an inhibitor for binding to its target entity. In some embodiments, an activating agent prevents or reduces aggregation of the target entity. In some embodiments, an activating agent stabilizes interaction of its target entity with another entity (e.g., a substrate protein, RNA, or DNA, a small molecule, peptide, or carbohydrate). In some embodiments, an activating agent binds to a target entity and increases the interaction of that target entity with another entity as compared with its interaction under comparable conditions absent the activating agent. In some embodiments, an activating agent-mediated increase in interaction of a target entity with another entity increases level and/or activity of that target entity as compared with its level and/or activity under comparable conditions absent the activating agent. In some embodiments, an activating agent binds to a target entity and decreases interaction of that target entity with another entity as compared with its interaction under comparable conditions absent the activating agent. In some embodiments, an activating agent-mediated decrease in interaction of the target entity with another entity increases level and/or activity of that target entity as compared with its level and/or activity under comparable conditions absent the activating agent.

In general, an activating agent may be or comprise a compound of any chemical class (e.g., a small molecule, metal, nucleic acid, polypeptide, lipid and/or carbohydrate). In some embodiments, an activating agent is or comprises an antibody or antibody mimic. In some embodiments, an activating agent is or comprises a nucleic acid agent (e.g., an antisense oligonucleotide, a siRNA, a shRNA, etc) or mimic thereof. In some embodiments, an activating agent is or comprises a small molecule. In some embodiments, an activating agent is or comprises a naturally-occurring compound (e.g., small molecule). In some embodiments, an activating agent has a chemical structure that is generated and/or modified by the hand of man. In general, an activating agent increases level or activity of one or more target entities present in and/or produced by a cell or organism. In some embodiments, a target entity is or comprises a polypeptide. In some embodiments, a target entity is or comprises a nucleic acid (e.g., a nucleic acid that encodes or regulates [e.g., by altering expression and/or activity of] a polypeptide). In some embodiments, a target entity is or comprises a carbohydrate. In some embodiments, a target entity is or comprises a lipid. In some embodiments, a target entity is or comprises an enzyme. In some embodiments, a target entity is or comprises a polypeptide involved in cellular trafficking.

Preventing and/or inhibiting the biological function of an NMDA receptor can be effected at the protein level but may also be effected at the genomic and/or the transcript level using a variety of molecules which interfere with transcription and/or translation of a receptor.

Therefore, non-limiting examples of antagonists that can be used according to some embodiments of the invention include small molecules, antibodies, inhibitory peptides, enzymes that cleave the polypeptide, aptamers homologous recombination agents, site specific endonucleases and RNA silencing agents.

According to specific embodiments, the antagonistic agent is an antibody. According to specific embodiments, the antagonistic antibody specifically binds at least one epitope of an NMDA receptor. According to specific embodiments, the antibody can cross the Blood Brain Barrier.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen. The antibody may be mono-specific (capable of recognizing one epitope or protein), bi-specific (capable of binding two epitopes or proteins) or multi-specific (capable of recognizing multiple epitopes or proteins).

Another form of an antibody fragment is a peptide coding for a single complementarity-determining region (CDR). CDR peptides (“minimal recognition units”) can be obtained by constructing genes encoding the CDR of an antibody of interest.

It will be appreciated that for human therapy or diagnostics, humanized antibodies are preferably used. Humanized forms of non-human (e.g., murine) antibodies are chimeric molecules of immunoglobulins, immunoglobulin chains or fragments thereof which contain minimal sequence derived from non human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Another agent which can be used as antagonist with some embodiments of the invention is an aptamer. As used herein, the term “aptamer” refers to double stranded or single stranded RNA molecule that binds to specific molecular target, such as a protein. Various methods are known in the art which can be used to design protein specific aptamers.

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, the antagonist of some embodiments of the invention can be an RNA silencing agent. As used herein, the phrase “RNA silencing” refers to a group of regulatory mechanisms [e.g. RNA interference (RNAi), transcriptional gene silencing, post-transcriptional gene silencing, quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene (e.g., NMDAR genes). In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated.

Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g. NAMDR) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to some embodiments of the invention, dsRNA is provided in cells where the interferon pathway is not activated.

According to an embodiment of the invention, the long dsRNA are specifically designed not to induce the interferon and PKR pathways for down-regulating gene expression.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAS are chemically synthesized as 21 mers with a central 19 bp duplex region and symmetric 2-base 3′-overhangs on the termini or different positions.

The strands of a double-stranded interfering RNA (e.g., an siRNA) may be connected to form a hairpin or stem-loop structure (e.g., an shRNA. Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Effects of these molecules on target simply can be followed the protein expression of the target, herein NMDAR receptors.

It will be appreciated that, and as mentioned here in above, the RNA silencing agent of some embodiments of the invention need not be limited to those molecules containing only RNA, but further encompasses chemically modified nucleotides and non-nucleotides and preparation methods.

According to another embodiment the RNA silencing agent may be a miRNA.

The term “microRNA”, “miRNA”, and “miR” are synonymous and refer to a collection of non-coding single stranded RNA molecules of about 19-28 nucleotides in length, which regulate gene expression. miRNAs are found in a wide range of organisms and have been shown to play a role in development, homeostasis, and disease etiology.

Although initially present as a double-stranded species with miRNA, the miRNA eventually becomes incorporated as a single-stranded RNA into a ribonucleoprotein complex known as the RNA-induced silencing complex (RISC) while the miRNA is removed and degraded.

Antisense is a single stranded RNA designed to prevent or inhibit expression of a gene by specifically hybridizing to its mRNA. Downregulation of a receptor can be effected using an antisense polynucleotide encoding the receptor or the receptor subunit.

The prior art teaches of a number of delivery strategies which can be used to efficiently deliver oligonucleotides into a wide variety of cell types [see, Molecular Cloning: A Laboratory Manual, 3nd Edition, Sambrook and Russel eds., Cold Spring Harbor Laboratory Press, Cold Spring Harbor 2001; Brown TA (Genomes. 2nd edition. Oxford, Wiley-Liss: 2002; Auld et al.—The assay Guidance Manual; https://www.ncbi.nlm.nih.gov/books/)]

Nucleic acid agents can also operate at the DNA level as summarized below.

Suppressing the biological function of a NMDA receptor can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the effects of the protein; a nonsense mutation, i.e., a mutation which introduces a stop codon in a protein, e.g., an early stop codon which results in a shorter protein devoid of the enzymatic activity; a frame shift mutation, i.e., a mutation, usually, deletion or insertion of nucleic acid (s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons; an insertion mutation, i. e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

Methods of introducing nucleic acid alterations to a gene of interest are well known in the art including site specific recombinases (e.g. the Cre recombinase and the Flp recombinase), PB transposases (e.g. Sleeping Beauty, piggyBac, Tol2 or Frog Prince), genome editing by engineered nucleases (e.g. mega nucleases, Zinc finger nucleases (ZFNs), transcription-activator like effector nucleases (TALENs) and CRISPR/Cas system) and genome editing using recombinant adeno-associated virus (rAAV) platform.

The NMDA antagonists and/or the agents of some embodiments of the invention can be administered to an organism per se, or in a pharmaceutical composition where it is mixed with suitable carriers or excipients.

The term “pharmaceutical composition” as used herein refers to any composition comprising at least one pharmaceutically active ingredient and at least one pharmaceutically acceptable carrier. The purpose of a pharmaceutical composition is to facilitate administration of a compound to an organism. Herein the term “active ingredient” refers to the NMDA receptor antagonist accountable for the biological effect.

Herein, the terms “pharmaceutically acceptable carrier” and a “physiologically acceptable carrier” which may be interchangeably used refer to a non-toxic solid, semisolid or liquid filler, carrier diluent or excipient of any type that does not cause significant irritation to an organism and does not abrogate the biological activity and properties of the administered compound.

Suitable routes of administration may, for example, include oral, rectal, transmucosal, especially transnasal, intestinal or parenteral delivery, including intramuscular, and subcutaneous.

In certain embodiments, the NMDA receptor antagonist and/or the agents described above are formulated for oral delivery. In certain embodiments, the NMDA receptor antagonist and/or the agents described above is formulated for injection. In certain embodiments the NMDA receptor antagonist and/or the agents are formulated for sustained release.

Sustained release allows delivery of a specific drug at a programmed rate that leads to drug delivery for a prolonged period of time. Sustained release by adjusting the speed of drug release can keep the concentration of the drug at a constant level in the blood or target tissue. When the drug is dissolved in the aqueous body fluid, it can be easily transported with the fluid to the target receptors. Some studies have shown that one method to achieve sustained drug release is by preventing drug molecules from entering completely the aqueous environment for a manageable period of time. This inhibition can be recognized by adjusting the degradation speed of a carrier, or by adjusting the diffusion rate of drug molecules over an insoluble polymer matrix or shell.

Conventional approaches for drug delivery to the central nervous system (CNS) include, bot not limited to: neurosurgical strategies (e.g., intracerebral injection or intra cerebro ventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). Alternately, one may administer the pharmaceutical composition in a local rather than systemic manner, for example, via injection of the pharmaceutical composition directly into a tissue region of a patient.

Pharmaceutical compositions of NMDAR blocker may be manufactured by processes well known in the art, e.g., by means of conventional mixing, dissolving, granulating, drage-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-inked polyvinyl pyrrolidone, agar, or alginic acid or a salt thereof such as sodium alginat.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended insuitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. NMDA receptor antagonist) effective to prevent, alleviate or ameliorate symptoms of a disorder (e.g., ageing, e.g., ageing related diseases) or increase the life span of the subject.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized.

Dosage amount and interval may be adjusted individually to provide that levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

A pharmaceutically active prodrug of the NMDA receptor antagonist may be used. As used herein the term “prodrug” refers to (i) an inactive form of a drug that exerts its effects after metabolic processes within the body convert it to a usable or active form, or (ii) a substance that gives rise to a pharmacologically active metabolite, although not itself active (i.e. an inactive precursor).

Depending on the severity and responsiveness of the condition to be treated, dosing can be of a single or a plurality of administrations, with course of treatment lasting from several days to several weeks or life long.

The amount of a composition to be administered will, of course, be dependent on the subject being treated, the severity of the affliction, the manner of administration, the judgment of the prescribing physician, etc.

The terms “comprises”, “comprising”, “includes”, “including”, “having” and their conjugates mean “including but not limited to”.

The term “consisting of” means “including and limited to”.

The term “consisting essentially (mainly) of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein can be utilized according to such related embodiments of the present invention. Thus, the following claims are intended to encompass within their scope modifications, substitutions, and variations to processes, manufactures, compositions of matter, compounds, means, methods, and/or steps disclosed herein.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples. They should, in no way be construed, however, as limiting the broad scope of the invention.

The Geroscience Hypothesis posits that fundamental ageing mechanisms are ‘root cause’ contributors to the increasing burden of disorders and diseases with advancing age that are responsible for the bulk of morbidity, mortality and health costs in the developed and developing worlds. These fundamental ageing processes include: (1) macromolecular dysfunction (e.g. protein misfolding and aggregation, decreased proteasome activity, DNA damage, telomere uncapping, increased advanced glycation end-products [AGEs], lipotoxicity and accumulation of bioactive lipids) and organelle dysfunction (altered nuclear membranes related to deficient lamin B, mitochondrial dysfunction leading to reduced fatty acid metabolism, higher glucose utilization, depletion of NAD+ and increased reactive oxygen species [ROS] generation, etc.), 2) chronic low grade ‘sterile’ (absence of bacteria, fungi, etc.) inflammation often accompanied by fibrosis, 3) stem, progenitor and immune cell dysfunction (including altered proliferative capacity and dysdifferentiation with failure to develop into functional mature cells, declines in ‘geroprotective’ factors [e.g. a-Klotho], contributing to stem and progenitor cell dysfunction) and 4) cellular senescence.

Unitary Theory of Fundamental Aging Processes hypothesizes that these fundamental ageing processes may be interlinked. Therefore, targeting any one fundamental ageing process (e.g. decreased proteasome activity, protein misfolding-aggregation, depletion of NAD+, cellular senescence) genetically or with drugs should affect many or perhaps all of the rest. Indeed, consistent with the Unitary Theory, senescent cells contribute to inflammation, fibrosis, DNA damage, development of protein aggregates, failed autophagy, lipotoxicity, mitochondrial dysfunction, depletion of NAD+, ROS generation and stem, progenitor and immune cell dysfunction.

Therefore, in another embodiment the present invention provides a medicament for ameliorating onset and/or progression and even treatment of the ageing and aging associated diseases by targeting more than one fundamental ageing process, including protein misfolding and aggregation, decreased proteasome activity, increased oxidated proteins, AGEs, lipotoxicity and accumulation of bioactive lipids and organelle dysfunction (mitochondrial and autophagy-lysosome disfunction), stem progenitor dysfunction, depletion of NAD+, cellular senescence comprising the step of administering a therapeutically effective amount of an NMDAR antagonist or a pharmaceutically acceptable salt or prodrug thereof.

### EXAMPLES

NMDA Inhibitors Degrade the p21, p27, p53 of the Cell Proteins.

In the present invention, it has been found that NMDA receptor antagonists (ketamine and memantin) cause decrease of the p21, p27, p53 (FIG. 1A) proteins of the HepG2, T98G and SH-SY5Y cell lines. It is known that these cellular proteins including p21, p27, p53 are classified as amyloidogenic IDPs and as many as 41% of the eukaryotic proteome is predicted to contain IDRs. The degredation is concentration dependent and starts within minutes which can be demonstrated by western blot in cell incubated with NMDAR antagonist for 10 minutes (FIG. 1B). The decrease in p21 content in the presence of cycloheximide indicates that the p21 protein is degredated rather than the inhibition of protein synthesis (FIG. 1C-D).

It was known by our laboratory and described previously that some of the NMDR blockers including ketamin inhibit Akt1 Ser473 phosphorylation which cause inactivation of Akt1 (FIG. 2A). Therefore to understand whether or not the inhibition of Akt1 Ser473 phosphorylation affect p21 degredation, allosteric phospho-AKT S473 inhibitor MK-2206 and specific inhibitor of PI3K which is upstream activator of phospho-AKT S473 were tested. FIG. 2B shows that although both MK-2206 and Worthmannin decrease phosphorylation of pAkt1 at Ser 473 much more than the ketamin did, they do not affect the p21 protein amount. Therefore effects of ketamin on degredation of p21 is not related with the PI3K/Akt1 pathway.

Proteasome Inhibitors Prevent the Degredation of p21, p27, and p53 Proteins Caused by NMDAR Blocker

Further experiments showed that the presence of protesome inhibitors either Mg132, lactacystine or both prevent or decrease the degredation of p21, p27, and p53 caused by the NMDAR blockers (lactacystine data was not shown) (FIG. 3A-C). These results indicate that the degredation of p21, p27, and p53 is related to the proteosome system, However, there was no effects of protesome inhibitors on the reduction of Akt1 phosphorylation that occurs in the presence of NMDAR blocker (FIG. 3D). Therefore, the effects of NMDAR blockers on Akt1 Ser473 phosphorylation and the p21 protein amount take place with different mechanisms and occur independently from each other.

NMDAR Inhibitors Also Degrade Phosphorylated Tau (p-Tau) and Serine Racemase (SR) and their Functions are Inhibited by Proteasome Inhibitors.

Tau is a major axonal microtubule-associated protein. Abnormal phosphorylation of tau interrupts its binding to microtubules and leads to the disruption of MTNs. Intracellular aggregates primarily consisting of phosphorylated tau (p-tau) are one of the hallmarks in Alzheimer's disease (AD). the accumulation of p-tau, a salient feature in AD, may impede the normal functioning of MTNs and cause a series of catastrophic cascades.

Serine racemase (SR) converts the free form of L-serine into D-serine (DS) in the mammalian brain. The DS functions as a co-agonist of NMDAR. The over-activation of the NMDA receptor leads to many neurological disorders like stroke, amyotrophic lateral sclerosis, and AD. NMDAR inhibitors (ketamin and memantin) degraded phosphorylated tau (p-tau) and Serine racemase (SR) and their functions are inhibited by proteasome inhibitors (FIG. 4A, B). The FIGS. 4A and B show only the results of HepG cells and ketamin. Similar results were obtained in T98G and SH-SY5Y cell lines and using memantin.

### NMDAR Blockers Increase Proteasome Activity

Therefore next the NMDA receptor antagonists on the proteosome activity was tested.

A commonly used method to measure proteasome activity is to make use of fluorogenic substrates. In this type of experiment small peptides are linked to a 7-Amino-4-methylcoumarin (AMC) group. Upon cleavage of this group by proteases such as the proteasome the AMC group becomes fluorescent and this signal can be measured overtime. These type of experiments are almost always performed with cell lysates or purified proteasome. Data from these type of experiments may therefore not be relevant in more complex environments such as whole cells or the situation in vivo.

Indeed, we demonstrated by a series experiments using cell lysate that NMDA receptor antagonists have no effects on any of the 3 subtypes of protesome activity including trypsin, chemotrypsin and caspase-like activity (FIG. 5. A-C.)

We developed (established the most effective concentration by adjusting digoxin-tween ratio) cell permeable versions of fluorogenic substrates to overcome these limitations and named “In-Cell Fluorogenic Proteasome Assay”. Indeed, using cell permeable versions of fluorogenic substrates, we demonstrated that NMDAR blocker increase the proteasome activity of trypsin, chemotrypsin and caspase-like activities statistically significantly (FIGS. 6 A, B, and C) in HepG cell. In addition, it was determined that NMDAR blockers increased the activities of trypsin, chemotypsin, and caspase-like enzymes depending on the amount of the drugs (FIG. 6D) (the results of HepG-chemotrypsin-ketamin experiment was shown only)

Further experiments using the Cell-Based Proteasome luciferase systems (Promega) which is the method applied in a living cell also showed that NMDA receptor antagonists increase Chemotrypsin proteasome activity similar to the results of In-Cell Fluorogenic Proteasome Assay (FIG. 7A) in HepG2. In addition no effects were found when Promega's luciferase system used with cell lysate for the chemotrypsin activity, the same as the results of the lysate based fluorogenic assay (FIG. 7B). Further experiments using the Cell-Based Proteasome luciferase systems (Promega) which is the method applied in a living cell also showed that NMDA receptor antagonists increase trypsin and chemotrypsin proteasome activities similar to the results of In-Cell Fluorogenic Proteasome Assay (FIG. 7C, D)

In addition to HepG2, NMDAR blockers increased the activity of trypsin, chemotypsin, and caspase-like activities in T98G glioblastoma and Hep3B hepatocellular carcinoma cell lines (FIG. 8A-C) (Memantin and Hep3B results were not shown).

In further experiment in addition to the ketamin, IU1 which was previously defined as proteaseome activator and the other NMDAR blockers including memantine and MK801 were tested and compared for their effect on proteasome. Mg132 was used as protesome inhibitor control. Results show that NMDAR blockers increased all of the three form of the proteasome activities at different levels in both HepG2 (FIG. 9A-C), and T98G cell line (FIG. 10A-C). IU1 is used as proteasome activator and increased all of the three form of the proteasome activities at different levels in HepG2 similar to ketamine (FIG. 9A-C).

This experiment also showed that Mg132 inhibits only the chemotyrpsin activity, but not the trypsin and caspase like activities. Contrary to the chemotrypsin activity, Mg132 did not inhibit trypsin activity of proteasome in HepG2, T98G and Hep3B cell lines (FIG. 9A-C, FIG. 10A-C).

NMDAR Blockers' Effect is not Related with Autophagy

Autophagy is another intracellular degradation system that derives its degradative abilities from the lysosome. The most well-studied form of autophagy is macroautophagy, which delivers cytoplasmic material to lysosomes via the double-membraned autophagosome. Autophagy is a multi-step process which includes not just the formation of autophagosomes, but most importantly, the flux through the entire system, including the degradation upon fusion with lysosomes. Other forms of autophagy, namely chaperone-mediated autophagy and microautophagy, occur directly on the lysosome.

Therefore, it was aimed to investigate whether the degredation of protein including p21 and p53 is related to an effect of NMDAR blocker on the autophagy-lysosome pathway.

One of the most experimentally straightforward method to monitor autophagic activity is the detection of microtubule-associated protein light chain 3 (LC3) protein processing. LC3 is the only known protein that is specifically associated with all types of autophagic membranes, including phagophore, autophagosome and autolysosome (a hybrid organelle formed by fusion of the autophagosome and lysosome). LC3 is specifically associated with the autophagosome. Upon the autophagy induction, the cytosolic LC3 (LC3-I) is conjugated to phosphatidylethanolamine (PE) to form lipidated LC3 (LC3-II). LC3-II binds to the expanding isolation membrane and remains bounded to complete autophagosome. Therefore, the amount of LC3-II correlates well with the number of autophagosomes, which is widely used as a marker for the autophagosome. Autophagy is a multi-step process which includes not just the formation of autophagosomes, but most importantly, the flux through the entire system, including the degradation of LC3-II upon fusion with lysosomes. Therefore, the expected result is that the LC3-II protein is degraded for complete autophagy. FIG. 11A shows that there is an increase in the amount of LC3-II amount in the presence of ketamin in the HepG2 cell stably expressing LC3. This result may indicate an increase in the autophagosome formation due to autophagy induction. However, total amount of LC3 (LC3-I plus LC3II) was not changed. Therefore, NMDAR blockers do not cause a lysosomal degredation.

In addition to LC3, levels of p62 (also known as SQSTM1/sequestome 1) can also be used to monitor autophagic flux. Although the proteasome pathway is also involved in p62 degradation, the main degredation mechanism of p62 is considered as autophagy. Therefore, its amount is generally considered to inversely correlate with the autophagic activity. Accumulation of p62 has been used as a marker for autophagy suppression, and similarly, a decreased p62 level indicates autophagic activation.

One critical point is that autophagy is a highly dynamic, multiple-step process that can be modulated at several steps. The amount of autophagosomes detected at any specific time point is a function of the balance between the rate of their generation and the rate of degradation though fusion with lysosomes. Accordingly, changes in amount of p62 could reflect either increased autophagosome formation due to autophagy induction, or a blockage in the downstream steps in autophagy, such as inefficient fusion or decreased autophagosome degradation. Thus, the mere detection of levels of p62 at a specific time point is insufficient for an overall estimation of autophagic flux, which refers to the complete process of autophagy including the sequestration of cargo within the autophagosome, the delivery of cargo to lysosomes and the subsequent release of the breakdown products. Thus, the p62 turnover needs to be investigated by the immunoblotting in the presence and with the absence of lysosomal degradation. Therefore, the experiment was conducted using the combination of leupeptin and NH4Cl to block the lysosomal degradation. In the results, although lysosomal protease inhibitors were found to increase the amount of p62, it was determined that ketamin had no effect on p62 amount (FIG. 11B).

The other basic protocol for the outophagy detection is monitoring the number of autophagosomes by visualizin LC3 puncta using flourescence microscopy. Upon induction of autophagy, LC3 becomes associated to the autophagosomal membrane. This change in the subcellular distribution of LC3 can be observed through either indirect immunofluorescence or by examining the signal of fluorescent protein tagged to LC3 (e.g., green fluorescent protein GFP-LC3). For this purpose the LC3 gene construct was stably inserted into the cell and after its continuous expression is achieved, the changes in the punctata in the presence of NMDAR blocker were investigated using LC3 specific antibody. The results showed that there was no change in the punctata density in the presence of NMAR blocker compared to the control (FIG. 11C).

To understand more directly the possible relationship of p21 and p53 degredation in the presence of NMDAR blockers with the autophagy, NMDAR blockers were tested in the presence of lysosome inhibitors. Results showed that while protesome inhibitor prevented p21 and p53 degredation due to NMDAR blockers as shown previously, lysosome activity blocker did not inhibit the p21 and p53 degredation in the presence of ketamin (FIG. 11D).

All results related with outophagy above clearly indicate that outophagy pathway is not involved in NMDAR-induced p21 and p53 degredation.

### The Ubiquitin Pathway is not Involved in the Effect of NMDAR Blocker on the Proteasome Activity

In the present invention, it has been found that NMDAR blocker increase proteasome activity and p21, p27 and p53 protein degredation. The ubiquitin dependent 26S proteasomal degradation process mediates the degradation of damaged and short-lived regulatory proteins. This active, ATP-dependent pathway involves a cascade of three different types of enzymes. Ubiquitin is first activated in an ATP-dependent reaction by an E1 ubiquitin-activating enzyme, to which it becomes attached by a thioester bond. Subsequently, the activated ubiquitin is transferred to the active site cysteine of the E2 ubiquitin-conjugating enzyme. Ubiquitin-protein ligase (E3), together with E2 catalyze the transfer of ubiquitin onto the protein that is destined for degradation by the 26S proteasome.

Therefore, it was aimed to investigate whether effects of NMDAR blockers are related with these steps. Further in this invention it is demonstrated by using a series of experiments that the effects of NMDAR blockers are ubiquitin-ATP-pathway independent.

This finding was supported by the following experiments; First, a model was developed to find inhibitor or inhibitors for the ubiquitin dependent degredation in the Hep3B cell line. For this purpose Hep3B cell expressing Ub-YFP genome were prepared. Ub-YFP is a system which was used to detect the ubiquitin dependent proteasome activity (26S). It is also known that Ub-YFP is not affected by the ubiquitin-independent protesome system. In further experiments, we tested molecules which are involved in different parts of the ubiquitination processes including PYR41 (Ubiquitin-activating enzyme (E1) inhibitor), NCC697923 (UBE2N E2 ubiquitin-conjugating enzyme inhibitor), SMER3 (ubiquitin ligase E3 inhibitor), SZL-P1-411 (Skp2 E3 ligase inhibitor), SKPIN C1 (Skp2 E3 ligase inhibitor), P005091 (USP7 inhibitor), Vialin A (USP4 and USP5 inhibitor), Spautin A (USP10 and USP13 inhibitor), ML 323 (USP1-UAF1 inhibitor), IU1 (USP14 inhibitor), and Mg132 (protesome inhibitor) for the inhibition of Ub-YFP degredation. The experiments revealed that PYR41 inhibits the Ub-YFP degredation and YFP expression appeared in flouresans microscope. MG132 was used as the inhibitor of the protesome activity (FIG. 12A). In the next experiment YFP protein amount was investigated in the presence of ketamin, Pyr41, and ketamin and Pyr41 together by western blot in Hep3B cell. FIG. 12B show that although Pyr41 increase YFP protein amount in cell, ketemin did not decrease the YFP protein amount.

It is known that degredation of p21 protein occur in both ubiqutin-proteasome system (UPS) (26S) and ubiquitin-independent protesome system (UIPS) (20S). In further experiment, the effects of PYR-41 on endogenous p21 and also activity of NMDAR blocker in the presence of PYR-41 in the HepG2 hepatocellular carcinoma cell (we know that Hep3B does not contain p21) were tested.

The experiment showed that PYR-41 increased the p21 protein amount and NMDAR blockers decreased p21 protein amount in both control cells and the cells having PYR-41 in similar manner (FIG. 13A, B).

In the further study, we made a p21-GFP constract and provided the expression of p21-GFP fusion protein in Hep3B in the presence of doxocillin. Similar to the endogenous p21 result in HepG cell, this experiment also showed that PYR-41 increased the p21 and GFP proteins amount and NMDAR antagonists decreased p21 and GFP amounts in both control cells and the cells having PYR-41 in similar manner (FIG. 14A-C) in Hep3B cell.

Therefore, results up to this stage showed that NMDAR blockers degrade p21 protein without being involved with the ubiquitation dependent processes

### NMDA Receptor Antagonists Elicit Increased Activity Through Increasing Proteasome 20S Subunit Activity.

The human 26S proteasome consist of a barrel-shaped 20S core particle (CP) capped by one or two 19S regulatory particles (RP). The 19S RP is responsible for 20S gate opening, substrate recognition and binding, unfolding, and threading of ubiquitinated substrates into the 20S CP. The 19S RP consist of two subcomplexes, the base that interacts directly with the 20S and a peripheral lid. The base is comprised of hexameric AAA-ATPase subunits, Rpt1-Rpt6, and tetrameric non-ATPase subunits, Rpn1, Rpn2, Rpn10, and Rpn13. Rpn13 serves as ubiquitin receptors that direct polyubiquitinated proteins to the proteasome. The lid is made of nine non-ATPase subunits; Rpn3, Rpn5-Rpn9, Rpn11, Rpn12, and Sem1. The lid, specifically the Rpn11 subunit, functions as a deubiquitinase. The Rpn subunits also create docking site (s) for other proteins including the proteasome associated deubiquitinating enzymes, USP14, and UCH37.

Further in this invention, it is demonstrated by using a series experiments that NMDA receptor antagonist increase proteasome activity through 20S subunit and 19S subunit of proteaseome has no role.

The first study conducted for this purpose was to investigate the effect of NMDAR blockers on protein degradation and the chemotrypsin activity of proteasome when Rpn13, which is an essential ubiquitin receptor subunit of the 19S regulatory particle, is inhibited.

RA190 is recently described as a highly selective inhibitor of the Rpn13. Firstly, the effect of NMDAR blockers on p21 degradation in the presence and absence of RA190 was investigated by the western blot. The findings showed that p21 amount was increased in the presence of RA190 which is most likely due to the inhibition of the UPS dependent degredation (FIG. 15A). However ketamin degraded p21 in the presence of RA190 as effective as in the control cell without RA190.

In further experiment, it was determined that NMDAR blockers increased the chemotrypsin activity of proteasome in the presence of RA190 as effective as in the absence of RA190. The results are shown as a line graph with one hour's measurments and as a bar graph at 15th minute (FIG. 15B,C). Only ketamin results were shown in these axperiments. Memantine results were very similar to the ketamin results in both p21 degredation and chemotrypsin activity tests.

The UPS includes the large family of Deubiquitinases (DUBs). DUBs mediate the deubiquitination of the proteolytic process of the UPS. Although the precise roles of proteasomal DUBs are not complately understood, they regulate the degredation of ubiquinated proteins by removing the ubiquinitated chains from the substrate. DUBs belong to the superfamily of proteases. The human genome encodes at least 98 DUBs which belongs to 6 subfamilies based on sequence and structural similarity. including ubiquitin carboxy-terminal hydrolases (UCHs), ubiquitin-specific proteases (USPs), JAMM/MPN domain-associated metallopeptidases (JAMMs).

There are three important DUBs associated with the 19S regulatory particle of proteosome, the JAMM family member POH1 (also known as RPN11/pda1/S13/mpr1), the USP family member USP14 and the UCHs family member UCHL5 (also known as UCH37). POH1 is a Zn-dependent metalloprotease, whereas USP14 and UCHL5 are cysteine proteases.

In further experiment, Gliotoxin was used to clarify the effects of NMDAR blockers on proteasome. It was described previously that gliotoxin inhibits the 19S proteasome regulator in vitro and in cells by targeting its essential deubiquitinase subunit Rpn11 also called PSMD14. PSMD14 is component of 19S regulatory particle of 26S proteosome. In addition, gliotoxin is also known as a noncompetitive inhibitor of the chymotrypsin activity of the 20S proteasome in high doses. Finding showed that the p21 amount was increased dose and time dependent in the presence of Gliotoxin which indicates the inhibition of the UPS dependent degredation (FIG. 16A). However NMDAR blockers degraded p21 in the presence of Glotoxin at the doses of 5 and 10 uM efficient as the control without gliotoxin (FIG. 16A). As indicated above that gliotoxin is a noncompetitive inhibitor of the chymotrypsin activity of the 20S, increasing dose of Gliotoxin decreased the p21 degredation by inhibiting the effects of ketamine. These results may indicate that effect of NMDAR blockers on p21 degredation occur through the Chymotrypsin activity of the 20S proteasome subunit but not trypsin and caspase-like effects. In the next study, effects of Gliotoxin on chemotrypsin activity of proteasome in the presence or absence of NMDAR blockers in low (5 uM) and high (25 uM) concentrations were investigated. FIGS. 16B and C show that chemotrypsin activity of proteasome was not statistically changed in comparision to the control at low dose of Gliotoxin, although it shows a tendency to increase. Importantly, NMDR blocker increased the chemotrypsin activity of proteasome as much as, even more in the presence of gliotoxin than the absence of it (FIG. 16B,C). Parallel to inhibition of p21 degredation, a high dose of gliotoxin (25 uM) inhibited the chemotrypsin activity of proteasome in the presence of NMDR blockers (FIG. 16D,E) (ketamin results were shown only). Surprisingly, degredation of p53 also occurred at a high dose of gliotoxin as much as control, differently from p21 (FIG. 16F). This may indicate that degredation of p53 occurs other than chymotrypsin activity. Immunoblot is completed using p53 and p21 antibodies together on the same membrane to ensure the results.

Capzimin is another inhibitor of PSMD14. Capzimin was tested in similar way as Gliotoxin. As is shown in FIG. 17A., Capzimin increased the p21 amount and NMDAR blockers could couse to degrede p21 in the presence of capzimin (FIG. 17A). As indicated previously PSMD14 is the part of the 19S proteasome subunit and necessary for ubiqutination dependent protein degredation. In further experiments, it was determined that NMDAR blockers increased the chemotrypsin activity of proteasome in the presence of capzimin as effective as in the absence of capzimin (FIG. 17B,C).

To further confirm and to be more specific that the NMDAR blockers act independently from the PSMD14 and therefore from the 19S subunit of the proteasome system, PSMD14 RNA expression was silinced using the lentiviral siRNA system containing PSMD14 spesific DNA coding (FIG. 18A). The result showed that NMDAR blockers decreased p21 protein in cell with PSMD14 silenced the same as in the control cell (FIG. 18B). In further experiments, it was determined that NMDAR blockers increased the chemotrypsin activity of proteasome more in the cell with PSMD14 silenced than in cells whose PSMD14 protein is not silenced (FIG. 18C,D).

As indicated above one of the other important DUBs which associate with the 19S regulatory particle of proteosome is USP14.

IU1 has previously been described to inhibit USP14 and increase the proteasome activity. Although not fully determined, there is a possibility that these two functions of IU1 are independent from each other. In this invention, it was determined that the presence of IU1 in the cell dramatically increases the amount of p21, while NMDAR blockers effectively reduce the amount of p21 in the presence of IU1 (FIG. 19). Previously in this invention it was determined that IU1 increases tyrpsin, Chemotrypsin and caspase-like activity of the protesome (FIG. 9A-C).

To further confirm and to be more specific for proving that the NMDAR inhibitors are acting as ubiqutin and 19S subunit independent way, USP14 gene expression was silinced using the lentiviral siRNA system containing USP14 spesific DNA coding (FIG. 20A). In addition, USP14 expression was increased in the cell using doxocillin inducible retroviral system (FIG. 20B). Results showed that the silencing of USP14 cause a decrease in the amount fo p21 (FIG. 20B). On the other hand p53 amount was increased in the USP14-silenced cell (FIG. 20B). However, as a result of the study conducted to investigate the effect of NMDAR blockers on p53 protein in USP14-silenced cell, it was determined that p53 decreased similar to the effect of NMDAR blocker in the control cell (FIG. 20B).

Importantly, while chemotrypsin activity increases in USP14 silenced cells relative to the control cell, the addition of the NMDAR blockers resulted in a much greater increase in the chemotrypsin activity in the USP14 silenced cell compared to the control cell (FIG. 20C,D). These results are like the results above indicate that the NMDAR blockers act independently from the 19S regulatory particle.

N-Ethylmaleimide which is described as a general inhibitor of deubiqutination enzymes including USP14, UCHL15 was used to further demonstrate that NMDAR blocker activate proteaseome independently from the 19S regulatory particle and ubiquitination processes. FIG. 21A shows that inhibiting whole DUB increased the p21 amount which may indicate decrease in UPS dependent p21 degredation. In addition, NMDAR blocker decreased p21 as efficient as the control cell which indicate that NMDAR blockers work with a DUB independent manner (FIGS. 21C and D).

All above experiments indicate that activity of the NMDAR blockers are through neither the ubiquitinilation nor the deubiqutination processes of UPS.

More interestingly, when the 19S regulatory particles including Rpn13, PSMD14, UCHL15 were inhibited by chemicals or silencing methods, effects of the NMDAR blocker on chemotrypsin activity of the 20S proteasome increased more than the 19S regulatory particles in the normal function.

Until at this stage in this invention, it has been shown that NMDAR blockers increase the trypsin, chemotrypsin and caspase like activity of protesome, this increase prevented by Lactacyctein and Mg132 which are the proteasome inhibitors, and the activation of proteasome and degredation of proteins by the NMDAR blockers occurred independently from UPS.

The next experiment was design to show that the activity of NMDAR blockers are directly related with 20S proteasome activity.

As indicated previously, the 26S proteasome consist of a barrel-shaped 20S core particle (CP) capped by one or two 19S regulatory particles. The 20S CP is a threonine protease that consists of four stacked rings. The two inner β-rings contain three catalytic subunits (β5, β2, and β1) that display chymotrypsin-like (CT-L), trypsin-like (Tryp-L), and caspase-like (Casp-L) activity, respectively. Unlike the 26S proteasome which primarily degrades the polyubiquitinated proteins, the 20S directly degrades misfolded, oxidatively damaged, and IDPs and IDR-containing proteins and does not require the unfoldase activity of the 19S base.

A number of other non-ATPase particles such as the 11S complex (PA28 α, β, and γ) and PA200 reversibly associate and regulate 20S. PA28 γ (REGgamma), a member of the 11S proteasome activators, have been shown to bind and activate the 20S proteasome to promote proteasome-dependent degradation of important regulatory proteins, especially p21. It is known that p21 can be degredated by UPS and UIPS way and UIPS degredation of p21 is regulated by the PA28 γ. In the next experiment, PA28 γ is slinced in HepG cell using Lenti siRNA vector cloned PA28 γ (REGgamma) siRNA sequence. FIGS. 22A and B show that in cells in which PA28γ was silenced, p21 degredation due to NMDAR blocker occured much less than the control cells. In addition, while proteasome activity decreased in PA28 γ silenced cells, the effect of NMDAR blocker on proteasome activity was also decreased in PA28 γ silenced cells compared to control cells (FIG. 22C, D). This finding indicate that NMDAR blockers activity is over 20S and its regulatory particles.

NMDAR Blockers are More Effective on Degradation of p21 Protein in the Presence of H₂O₂

It is described previously by different researchers that oxidatively modified proteins are known to be more susceptible to proteolytic degradation by the proteasome than native proteins. In addition, it was described that the 20S proteasome is more resistant to oxidative stress than the 26S proteasome as the 20S complex can maintain activity even upon treatment with moderate to high concentrations of H₂O₂, whereas the 26S proteasome is much more vulnerable likely because of the observed separation of the 19S particle from the 20S core in the presence of H2O2 (Ishii et al. 2005). Therefore next study aimed to reveal the effect of NMDAR inhibitors on the oxidatively damaged proteins. For this purpose different concentration of H₂O₂ tested on the p21-GFP genome-inserted HepG2 cell to find out whether H₂O₂ has an effect on GFP expression hence p21. It was determined that H₂O₂ increased the GFP signal dramatically at a 12 hour period and a dose of 0.2 mM (FIG. 23A). Likewise, at the same time and dose, H₂O₂ increased the endogenous p21 of the cell dramatically (FIG. 23B). Figure also shows, crucially, that NMDAR blockers reduces endogenous p21 much more effectively in the presence of H₂O₂.

This result may indicates that NMDAR blocker are much more effective on p21, which oxidatively modified in the presence of H₂O₂ compared p21 of the control cells. However the result also may indicate that the separation of the 19S particle from the 26S proteasome complex in the presence of H₂O₂ may cause the NMDAR blocker increase 20S protesome activity, similar to the results of 19S particle inhibitors' effects described above.

### Effects of NMDAR Blockers on Aging and Aging Related Phenotypes

The contribution of oxidative stress in age-related organ changes seems generally agreed that increases in ROS accompany aging, leading to functional alterations, increased incidence of disease, and a reduction in life span. Upon oxidative damage, proteins unfold and expose hydrophobic regions which makes them prone to aggregation.

Therefore, clearance of misfolded proteins are critical for cell survival because misfolding alters a protein's three-dimensional structure, impairing its biological activities and increasing its propensity to form toxic aggregates.

Modulation of protein concentration via regulation of the proteolytic machineries have long been validated as promising milieu for the development of treatments for ageing and ageing related different human diseases such as neurodegeneration, cancer, and autoimmunity, ageing and ageing related diseases

The proteasome is the cell's first defense mechanism against accumulating proteotoxic stresses induced by oxidative damage.

Because the ability to mount homeostatic adaptive responses declines with age, resulting in the decline of overall protein homeostasis, measuring the adaptive response to oxidative stress and the elimination of damaged proteins by the 20S proteasome may potentially provide useful biomarkers and prognostic data to streamline therapeutics for aging patients.

Therefore, in the next for this invention, the effects of NMDA blockers, which activate the 20S proteasome, on aging of cells and organisms were investigated.

### Effects of NMDAR Blockers on Aging and Aging Related Phenotypes

### On Primary Fibroblast Cells

First, primary human fibroblast cells were divided into two as control and NMDAR added group at the 4th passage and followed under the same condution until the 10th passage.

Cellular senescence is defined as a complete and irreversible loss of replicative capacity in primary somatic cells. (hayflick 1961). Therefore, MTT assay is used to measure cellular metabolic activity as an indicator of cell viability and proliferation in cells set up at passage 8. It is found that the proliferation was higher in the cells treated with ketamin or memantine compared to the control (FIG. 24A).

The one of the other major feature of senescent cell is an increase in Senesence-associated β-galactosidase (SA β-gal) activity.

Therefore cellular senescence were investigated by SA β-gal staining in cells at passage 10. It is found that control cells have 2.6 folds more SA β-gal stained cells compared to the cells treated with ketamin and 1.8 fold more SA β-gal stained cells compared to the cells with memantine, p=0.001 and 0.02, respectively (FIG. 24B).

In another experiment, the amount of intracellular lipid droplets as indicator of fat metabolism were investigated in senescent cells with nile red staining in cells at the late passage. Nile red is a lipophilic stain which stain intracellular lipid droplets and proteins. Lipid droplets are highly dinamic cellular organelles involved in the storage and the metabolism of neutral lipids.

FIGS. 24C and D show that cells with either ketamine or memantine were found to give more flourescence than those without drugs (P=0.0001). These results may indicate that senescent cells not only lose their proliferation abilities but also their fat metabolism becomes insufficient.

Effects of NMDAR Blockers on Aging and Aging Related Phenotypes on C. elegans

C. elegans is a free-living multicellular organism. Clear age-dependent human like physiological changes at the tissue, cellular, and molecular levels make C. elegans a valuable model for research in the field of aging and life span studies (Ambros, 2006). Accordingly, it was investigated how NMDAR blockers affact the life span of C. elegans. The use of drugs in the C. elegans related studies has been applied in different ways. The drugs were either applied to the LB medium with bacteria OP50, before spreading onto Nematode Growth Medium (NGM) plates (LB medium method), or to the NGM with live (NGM live method) or dead bacteria (NGM dead method), or spotting the drug solution to the surface of plates directly (spot dead method), or growing the worms in liquid medium (liquid growing method) (Ambros, 2006). Liquid growing method achieved the best absorption efficiency in worms. The drug concentration within worms were comparable with that in mice, providing a bridge for dose translation from worms to mammals. Therefore in this invention all the experiements related with the C. elegans were completed in liquid medium. In order to understand the effectiveness of the drug according to the onset time, the memantin was added to the medium on the 1st, 7th and 14th days in a 24-day period and the number of viable worms were determined on the 20nd day.

The results show that on 20th day, almost 3 times more C. elegans survived in the presence of memantine than the control when memantin started to be added on day 7 or day 1 (FIG. 25A). When the memantin started on the 14th day, 67% more C. elegans survived in the presence of memantine than the control on 20th day(FIG. 25B). In subsequent molecular and phenotypic studies of C. elegans, the memantine was started on day 7.

In C. elegans, features associated with aging could result in less active, uncoordinated movements, torpor, and accumulation of auto-fluorescent deposits in cells. Therefore, the movements of the C. elegans grown at 20° C. on standard nematode growth medium(NGM) plates shaked at constant speed at 200 rpm were recorded for 5 minutes on the 18th day. All C. elegans in plate well were individually followed and counted by three different eyes for 5 minutes. In the results, at least 3 times more non-moving C. elegans were observed in controls than those receiving memantine (P=0.5 E-6) (FIG. 25 C). One of the basic movement of C. elegans in liquid is crawling while the other is swimming. Previously described that swimming is more energetically demanding for C. elegans than the crawling motion. In addition it was stated that having the ability to swim is related to locomotory fatigue, muscle mitochondrial oxidation, a transcriptional oxidative stress response, and changes in carbohydrate and fat metabolism. It was also known that the swimming ability of C. elegans is also related to pharyngeal, intestinal, neuronal functions and strong muscle into old age. The result shows that the number of C. elegans that can swim is 2,4 times higher in plate with memantine than in plate without memantine (P=0.0007) (FIG. 25C).

The age-related reduction in fat oxidation, therefore, may promote the accumulation of total and central body fat. Adipogenesis and lipid accumulation during aging have a great impact on the aging process and the pathogenesis of chronic, age-related diseases.

In the next study, the effects of NMDAR blocker on lipid accumulation and central body fat and thus the fat metabolism in C. elagans was investigated. As mentioned above Nile red is a lipophilic stain which stain intracellular lipid droplets in cell. Therefore, in the presence of memantine and control C. elegans groups on the 19th day, the amount of fat in the living organisms was investigated by Nile Red staining.

The results of the study show that, on the 19th day, lipid accumulation in the control C. elagans group was more than 2-fold higher than the C. elegans grown in the presence of memantine (p=0.003) (FIG. 25D, E).

Effects of NMDAR Blockers on NADP+ and NADPH+ in C. elegans

Coenzyme I (nicotinamide adenine dinucleotide, NAD+/NADH) and coenzyme II (nicotinamide adenine dinucleotide phosphate, NADP+/NADPH) are involved in various biological processes in mammalian cells. Although NAD+ and NADP+ are synthesised in sufficient amounts under normal conditions, shortage in their supply due to over consumption and their decreased synthesis has been observed with increasing age and under certain disease conditions. Several studies have proved that in a wide range of tissues, such as liver, skin, muscle, pancreas, and fat, the level of NAD+decreases with age. The ratio of NAD+/NADH and NADP+/NADPH indicates the cellular redox state. A decrease in this ratio affects the cellular anaerobic glycolysis and oxidative phosphorylation functions, which reduces the ability of cells to produce ATP (She et al. 2021).

The molecule exists in cells in reduced (NADPH) and oxidized (NADP+) forms reflecting the redox state of the cell. An important cellular role for NADPH is to provide the reducing power for reductive biosynthesis of macromolecules including fatty acids and complex lipids, proteins, and nucleotides. NADPH also plays an important role in the control of cellular redox state. In this latter function, NADPH provides reducing equivalents for reduction of oxidized glutathione (GSSG) to the reduced form (GSH), a reaction catalyzed by glutathione reductase. GSH is an important intracellular reductant and plays an important role in cellular anti-oxidant defense.

Therefore, the amount of NADP+ and NADPH+ were investigated in C. elagans. The results of the study show that, on the 19th day, 2-folds more NADP+ was detected in C. elagans grown in medium with memantine compared to the C. elegans grown in medium without memantine (FIG. 25F, G).

### Effects of NMDAR Blockers on Aging and Aging Related Phenotypes on Mice

### Clinical Frailty Tests

In subsequent studies, mice were used to investigate the effects of NMDAR blocker, since all tissues and activities are substantially the same or similar to those of human.

Ten mice, one year old, were supplied for the study. The mice were divided into two groups and one added memenatine to their drinking water, while the other group was designated as the control group. Mice were followed until they were 2 years old. Clinical frailty tests were investigated and compared in the two groups of mice at 22 months. The simplest and most easly determined findings of clinical signs of deterioration in aging mice are the evaluation of changes in mouse skin. Indeed, the color change and condition of mouse fur in 22 months of age with and without memantine was very different in favor of those who received memantine (FIG. 26).

Mice are used to model of human aging and many motor disorders, both somatic and central nervous system in orgin. For all of these models, a full assessment of their motor deficients must include spesific test of strength.

Kondziela's inverted screen test is one of tests which examine the muscle strength using all four limbs. Four limb-hanging test which is the Kondziella's inverted screen test represents a method to assess muscle strength using all four limbs grip and to determine the general condition during time. This test makes use of a wire grid system to noninvasively measure the ability of mice to exhibit sustained limb tension to oppose their weight. As a result, the test can provide important information about age-related neuromuscular decline.

The holding time should not be below a certain score to determine the mice to be tested in order to distinguish those have any motor disorders (Table 2). Since the duration of holding times of all mice were over the score 2, all mice qualified for the other applications of the Kondziela's inverted screen test. One application of the Kondziela's inverted screen test is the time between the mouse falling from the wire screen after the screen is inverted. This test mainly shows the muscle strength of the fore and hind feet and paws as a whole. The results showed that mice receiving memantine could stay in the inverted frame for twice as long (P=0.006) (FIG. 27A).

Another test that shows the muscle strength more precisely in the Kondziela's inverted screen test is the mobility time of the mouse in the specified time after inverting the screen. As shown in the FIG. 27B, mice receiving memantine were able to move more than twice as much as those who did not receive memantine (P=0.007).

Four fingers grip time test which is part of the Kondziella's inverted screen test represents a method to assess muscle strength using all four fingers grip and to determine the general condition during time. As a result of four fingers grip test, it was determined that the group of mice that received memantine constantly used fingers and stayed on the wire for more than two times than the group that did not received memantine (P=0.001) (FIG. 27C).

Another grip test that measures muscle strength more finely is forelimb grip strength test. Many biomarkers related to aging have been identified in human. Importantly, the grip tests performed on humans has been defined as a highly predicitive indicator that can show disabilities, diseases and deaths that may occur due to aging (Iconaru et al., 2018; Sousa-Santos and Amaral, 2017) In this test a mouse is held by the middle base of the tail and lower it to allow it to grasp the weight which is a ball of tangled fine gauge stainless steel wire. This test is carried out by increasing the weight, investigating the weight that the mouse can carry or keeping the weight constant and how long the mouse can carry the weight.

In the test by determining how long mice can bear a fixed weight, we determined that the mice that received memantine could hold the weight with their for limbs for four times longer than those who did not receive memantine (P=0.003) (FIG. 27D).

Another test showing locomotor activity in mice is Open Field Maze (OFM). The OFM is one of the most widely used platforms which allow the researcher to measure behaviors ranging from overall locomotor activity to anxiety-related emotional behaviors. The first and most important specific parameter to measure in the OFM is total ambulatory distance. It is considered that an increase in the total ambulatory distance in mouse not only shows good locomotor activity, but also shows the exploratory feature of the mouse.

When the five minute recordings of the movements of mice in the OFM apparatus, it was determined that mice receiving memantine walked nearly two times more than mice not receiving memantine (FIG. 27E) (P=0.007).

The rearing activity test is the part of the OFM tests. Rearing behavior consists of subject animals standing on both hind paws in a vertical upright position. An increase in these test scores indicates that the locomotor activity increases and anxiety decreases. In the on going experiment, total rearing activity test showed that mice receiving memantine had twice as much rearing activity than mice who did not receive memantine (P=0.048) (FIG. 27F).

In addition to locomotor activities, the OFM remains one of the most widely applied techniques in rodent behavioral research.

As mentioned above, total ambulatory distance test shows the anxiety state of the mouse in addition to showing locomotor activity. Another test that shows anxiety in mice and is included in OFM tests is Thigmotaxis. This test consist of different measurements such as Central zone time, Peripheral zone time, Central escape latency, Central latency time, and Central zone entrance in the OFM.

In the next experiment, when observing the 5-minute movements of the mice in the regions within the OFM, it was determined that mice receiving memantine remained in the central region 4 times more than mice not receiving memantine (P=0.01) (FIG. 30G).

In addition it was determined that mice not receiving memantine remained in the peripheral regions more than mice receiving memantine (p=0.01) (FIG. 27H). In addition, Central escape latency, Central latency time, and Central zone entrance studies, which are other studies of OFM, were also conducted. Although the means of all them differed at least twice, the p value was determined to be greater than 0.05 in all three studies (P values are 0.4, 0.07,and 0.08, respectively) (FIG. 27 I,J,K).

### Effects of NMDAR Blockers on Molecular Age-Related Changes on Mice

Proteins are one of the major targets of oxygen free radicals and other reactive species. A constant accumulation of oxidized proteins takes place during aging. Oxidation of proteins leads to a partial unfolding and, therefore, to aggregation. Protein aggregates impair the activity of cellular proteolytic systems (proteasomes), resulting in further accumulation of oxidized proteins. In addition, the accumulation of highly crosslinked protein aggregates leads to further oxidant formation, damage to macromolecules, and, finally, to apoptotic cell death. Furthermore, protein oxidation seems to play a role in the development of various age-related diseases, for example, neurodegenerative diseases.

Oxidative modification of proteins by oxygen free radicals and other reactive species such as hydroxynonenal occurs in physiologic and pathologic processes. As a consequence of the modification, carbonyl groups are introduced into protein side chains by a site-specific mechanism. The carbonyl groups in the protein side chains are derivatized to 2,4-dinitrophenylhydrazone (DNP-hydrazone) by reaction with 2,4-dinitrophenylhydrazine (DNPH). Therefore it is possible to determine the side chains of protein converted to DNP-hydrazone using anti-DNP Antibody by Western Blotting after the DNP-derivatized protein samples are separated by polyacrylamide gel electrophoresis.

In the next study, oxidation status of proteins were investigated in tissues of 24 month old mice receiving memantin for 12 months and control mice. Proteins were converted to DNP-hydrazone, and then western blot was made. Oxidized proteins were found to be reduced in different amounts in tissues studied, including liver, brain, kidney, skin and pancreas of mice who received memantine compared to those who did not (FIG. 28). No significant change was observed in oxidation of protein obtained from the lung tissue. Western blots were repeated with proteins from different members of the two groups and similar results were obtained.

As mentioned above, amyloid is insoluble misfolded/unfolded fibrillary proteins, which aggregate after the loss of a protein's native structure. Aging and aging associated diseases are strongly associated with accumulation of Amyloid deposits occur in the in organs. Although, amyloid fibrils are mainly determined in the brain, it has been shown that amyloid fibrils accumulation occurs in different organs such as liver and kidney (Su et al. 2012; Tasaki et al. 2021).

In the next study, the effects of NMDAR blocker on amyloid accumulation in brain, liver, and kidney of mice with the presence of memantine and control mice groups at 22 months were investigated by amyloid staining.

According to our results, although the most significant difference in the amount of amyloid occurred in the brain tissue, a statistically significant decrease in the amount of amyloid was also determined in the liver and kidney of the group of mice having memantine (FIG. 29A-C).

In addition, we investigated the effect of ketamin on beta-amyloid precursor protein in Hep3B and SH-SY5Y neuroblastome cells expressing APP695 (beta-amyloid precursor protein) with western blot. FIG. 30 shows that beta amyloid protein is decreased in the presence of ketamin in both cells.

Discoloration are symptoms of skin ageing. They are connected with the presence of melanin and lipofuscin. The skin is composed of epidermal units that are responsible for production and distribution of melanin. In physiological conditions melanin synthesis occurs in melanocytes. Its enzymatic and structural elements are organized separately in a process resembling lysosome formation. Melanocytes are responsible for the production of proopiomelanocortin peptides (POMC), cytokines, nitric oxide (NO), prostaglandins, leukotrienes, which behave in a paracrine or autocrine way in keratinocytes.

Lipofuscin is a waste material of intracellular structures that gets accumulated in lysosomes, between postmitotic cells. An amount of lipofuscin rise with age in postmitotic cells and that is why it is called an age pigment or hallmark of aging. A chemical analysis of lipofuscin granules revealed the presence of protein and lipid compounds (20-50% and 30-70%, respectively). Protein content consists of different amino acids, while lipid includes triglycerides, free fatty acids, cholesterol, and phospholipids. Carbohydrates account for 4-7% of lipofuscin content. In skin and liver, the tissues where melanin and lipofuscin are most frequently detected, the amounts of melanin and lipofuscin were statistically decreased in mice treated with memantin (FIGS. 31A and B).

The age-related reduction in fat oxidation, therefore, may promote the accumulation of total and central body fat. Levels of lipids in adipose tissue, liver, and skeletal muscle change significantly during aging (Trayssac et al. 2018).

In the next study, the effects of NMDAR blocker on lipid accumulation in liver, brain, muscle, lung, skin, pancreas, and kidney of mice with the presence of Memantine and control mice groups at 22 months were stained with Nile red and investigated.

It was observed that the amount of lipid in the tissues of mice group receiving memantine including liver, brain, muscle, lung, and skin was less than that of the control group. No clear difference was observed in pancreas and kidney (FIG. 32A-E).

In above studies, we have shown that memantine increase the life span of C. elegans, as well as increase their muscle strength. In mouse studies, although, we have shown that memantine phenotypically increases muscle strength, decreases fat deposition in skin and muscle, and decreases oxidation of proteins, which are markers of the aging, we did not investigate the direct effect of memantine on mouse lifespan.

However, robust results in detail have been published showing that memantine increases mouse survival.

### NMDAR Blockers Increase Mice Lifespan

In the study published and patented by Anthony FUTERMAN and Andres David KLEIN. they showed that the life span of mice strains including A/J (AJ), C3H/HeJ (C3H), DBA/2J (DBA), and C57BL6/JolaHsd (C57) with Gaucher diseases symptoms was dramatically extended by memantine (FIG. 33A) and MK801 (another NMDR blocker) (FIG. 33B). FIG. 33C shows that increasing the memantine dose by 10 fold increases the life span of mice even more. They also showed that muscle strength were much better in mice receiving memantine with Kondziela's inverted screen test (FIG. 33D).

FIGS. 33 A-D are figures adapted from the patent of futerman et al. US 2170273917 A1.

In their study, they created Gaucher disease (GD) on 15 inbred mouse strains from diverse phylogenetic origins by inhibition of acid beta-glucosidase (GCase) using the conduritol B-epoxide (CBE). GD, one of the most common lysosomal storage disorders (LSDs), is caused by mutations in GBA1, the gene encoding the lysosomal hydrolase, acid β-glucosidase (GCase). The resulting GCase deficiency causes accumulation of the glycosphingolipids (GSLs) glucosylceramide (GlcCer) and its deacylated forms, within the lysosomes of cells.

They also demonstrated a correlation between the amount of CBE injected into mice and levels of accumulation of the Gcase substrates, GlcCer and GSLs, and show that disease pathology, indicated by altered levels of pathological markers, depends on both the levels of accumulated lipids and the time at which their accumulation begins.

They showed that by using memantine in all mice with GD symptoms, their lifespan extended and their motor coordination improved significantly. On the other hand, they showed that memantine had no effect neither on Gcase inhibition or the level of the Gcase substrates, GlcCer and GSLs, which led to the emergence of symptoms of GD (see patent, United States, Patent FUTERMAN et al, Application Pub. No.: US 2017/0273917 A1 and date Sep. 28, 2017, and Klain et al 2016).

Accumulating evidence indicates that GlcCer and Glc-Sph accumulation is directly related to aging. Recent findings in PD patients and aging control cases have shown an age-dependent reduction in GCase activity and an elevation of GlcCer and Glc-Sph.

Parallel biochemical analyses revealed a change in lipid metabolism in the aging brain may precede or may be part of abnormal protein homeostasis and may accelerate pathophysiological processes in age-related neurodegenerative disorders.

These data are important for the popular view that proteinopathy is the only trigger for aging related disease including neuronal demise in PD because altered GSL homeostasis may parallel or even precede changes in protein aggregation.

Therefore, in fact, Futerman et al. disrupted protein homeostasis network by inhibiting protein degredation capacity by inhibiting GCase actually provided premature aging associated with interferes with crucial signaling pathways and is often associated with multiple human diseases including PD.

Supporting this it was shown that C2-ceramide and C6-ceramide increase senescence as revealed by increased the amount of p53, and p21 which are important members of the MDM2-p53-p21Cip1 pro-survival pathway of mammalian cells.

### NMDAR Blockers as a Senolytic Drug Group

In addition to functions mentioned above, another function of NMDAR blocker is that they can be a senolytic group of drug.

Ablation of senescent cells have been postulated as a promising therapeutic approach to target the ageing phenotype and, thus, to prevent, delay or mitigate ageing-related diseases. The aim with senolytic compounds is to rejuvenate organisms by selectively killing senescent cells and their efficacy is based on the ability of senescent cells to resist apoptosis. These cells exhibit upregulation of senescence-promoting transcription factor cascades, in some cases involving p16INK4a-retinoblastoma protein (Rb), in others, p53 and p21CIP1, or the PI3K-AKT-ceramide metabolic pathway, both of these pathways, or other pathways.

As shown in this invention above (FIG. 1), NMDAR blockers decrease the amount of p21, p53 and ppAkt1. Result of this invention also showed that although NMDAR blockers did not cause a change in the amount of pRb protein, they caused a dramatic decrease in the amount of phospho pRb (FIG. 34).

### NMDAR Blockers Increase Beta-Actin Degredation

Previously described by Tedeschi and Dupraz (Tedeschi and Dupraz 2019) that the actin turnover provide an increase in axon regeneration which prevent and/or treat neurodegeneration caused by pathological insults. Result of this invention also showed that NMDAR blockers increase the degredation of beta-actin protein dose dependent (FIG. 35).

### NMDAR Blockers Maintain the Sternness Characteristics.

The present invention provides compositions and methods which maintain the sternness characteristics.

We investigated the differentiation of stem cells in the presence of memantine after cell passages using the simple differentiation method using the DMEM/F12, 10% FBS without coculture of mouse fibroblast (Yoo et all. 2020).

Cell were harvested at day 14 for western blot and the protein expression amounts of the differentiation markers cd44, cd73, and cd90 were investigated. The amount of cd44, cd73, and cd90 have increased on the 14th day according to the control cells (non-passaged) and this increase in markers in memantine treated cell is statistically significantly less than the cell not memantine treated, on day 14 (FIG. 36A-C). Another indicator of the stem cell differentiation is the determination of the amounts of lipid droplet in the cell. Fourteen-day cell passage is caused to increase lipid in the cell. It was observed that memantine treatment has prevented the amount of lipid droplet (FIG. 36D). In addition, present invention increase the lifespan of human mesenchymal stem cells. FIG. 36E shows the MTT test of the cells on the day 21 (P<0,034).

### Materials and Methods

### Cells

HepG2 and Hep3B human hepatocellular carcinoma cell lines, T98G human glioblastoma cell line and SH-SY5Y human derived neuroblastoma cell line were used in these experiments. The cells were grown in DMEM supplemented with 10% FBS, 1% Pen/Strep, 1% L-glutamine. Human primary bone marrow mesenchymal stem/stromal cells (BM-MSCs) (Cat. No: PCS-500-012™) and Human primary dermal fibroblasts (DFs) (Cat. No: PCS-201-012™) (ATCC, Manassas, VA, USA) were gift by Dr. Ekim Taskiran; passage 3 cells were used. Culture medium (DMEM 10% FBS, 1% Pen/Strep, 1% L-glutamine). Human Umbilical Cord Mesenchymal Stromal Cell (HUCMSCs) was gift by Dr. Ozgur Cinar and Dr. Ferda T. Celikkan. Human HUCMSCs was isolated, propagated and banked in accordance with a cGMP protocol (Can et al. 2015). HUCMSCs were grown in DMEM/F12, 10% FBS, 1% Pen/Strep, 1% L-glutamine. Passage 3 cells were used in the experiments. All cells were incubated at 37° C., 5% CO2 conditions.

### Antibodies

p21 p27, Akt1, and pAkt1 (Cell signalling); p53, p62, Serine racemase, retinoblastoma protein (Rb, phospho pRb, GFP, APP695 (beta-amyloid precursor protein), GAPDH, B-actin (Santa cruz); tau, pSer199 tau (Thermo fisher scientific); LC3 (Sigma Aldrich); PA28 γ (REGgamma), PSMD14, USP14, cd44, cd73, cd90 (Solarbio), YFP (Biovision). All antibodies were used in accordance with the manufacturer's recommendations.

### Chemicals and Kits

Mg132, RA190, Gliotoxin, Capzimin (Santa cruz); Lactacystine, Ketamin hydrochloride, Suc-LLVY-AMC, P005091, Vialin A, Spautin A, ML 323 SKPIN Cl, SZL-P1-411, SMER3, C1, NCC697923, PYR41, IU1 (Cayman Cehimacal); Memantine hydrochloride, dizocilpine maleate (MK-801), Worthmannin, MK2206, cycloheximide, Cell Proliferation Kit (MTT) (Sigma-Aldrich); Z-LLE-AMC, and Ac-RLR-AMC Ac-RLR-AMC (AdipoGen Life science); Cell-Based Proteasome luciferase systems (Promega); Senescence β-Galactosidase Staining (Cell signalling); OxyBlot-Protein Oxidation Detection Kit (EMD Millipor); Leupeptin, N-Ethylmaleimide, Nile red, Melanin and lipofuscin stain (with Nile blue), Amyloid Staining Kit (Improved Stores Congo red), NADP(H) Content Assay Kit (Solarbio). All chemicals and kits were used in accordance with the manufacturer's recommendations and different uses were indicated. Memantine, if not differently specified, has been used in the range of 0.1-0.2 mM in vitro. Ketamin, if not differently specified, has been used in the range of 0.5-1 mM in vitro. MK801, if not differently specified, has been used in the range of 0.1-0.2 mM in vitro.

### Western Blot Analysis.

Tissues or cell pellets were lysed in 20 mM Tris (pH 7.5), 150 mM NaCl, 1 mM EDTA, 1 mM EGTA, and 1% Triton X-100 in the presence of a protease inhibitor cocktail (Cell Signalling Tech. Massachusetts, USA). Fifty micrograms of lysate was resolved on 8%-12% Bis-Tris PAGE gels (Invitrogen) and transferred to nitrocellulose membranes (Bio-Rad California, USA). The membranes were blocked at room temperature for 1,5 h in phosphate-buffered saline (PBS) supplemented with 5% skim milk and 0.1% Tween-20 and incubated overnight at 4C° with primary antibody. Antibodies described were used for immunoblotting.

### Proteasome Activity Assay.

The activity of the 26S proteasome was measured by following steps. Aliquots of 2×106 cells were lysed with glass beads (Sigma) at 4° C. in lysis buffer containing 50 mM Tris-HCl (pH 7.5), 5 mM MgCl2, 1 mM DTT and 250 mM sucrose, and the protein concentration of cleared supernatants was measured by BCA protein assay reagent (Pierce). Ten micrograms of total protein was diluted with 26S proteasome assay buffer in a 96-well microtiter plate (BD Falcon), and incubated with fluorogenic substrates including Suc-LLVY-AMC, Z-LLE-AMC, and Ac-RLR-AMC Ac-RLR-AMC (Cayman-U.S.A.) were used to measure chymotrypsin, caspase-, and trypsin-like proteasome activity, respectively. Fluorescence released by AMC fluorescence was monitored on a microplate fluorometer (Biotek instrument-synergy HT) every 5 min at 37° C. for 1 h.

Proteasome activity assay using the Cell-Based Proteasome luciferase systems (Promega) which is the method applied in a non-lysate living cell. For a 96-well plate format, 8×103 cells per well were seeded. 24 hours later medium was changed with NMDAR blocker containing medium. Two hours later the Proteasome-Glo™ Cell-Based Reagent was preapared according to the kit protocol and 100 μl of Proteasome-Glo Cell-Based Reagent was added to each 100 μl of sample and appropriate controls as needed. After incubation at room temperature for a minimum of 10 minutes the luminescence of each sample were measured in a plate-reading luminometer (Biotek instrument (Synergy HT)) as directed by the manufacturer.

### Proteasome Activity Assay Using In-Cell Fluorogenic Proteasome Assay:

We developed cell permeable versions of the fluorogenic substrates in a non-lysate living cell assay in which the most effective concentration of digoxin-tween ratio is established to measure the proteasome activity effectively. For a 96-well plate format, 8×103 cells per well were seeded. 24 hours later medium was changed with NMDAR blocker containing medium. Two hours later 100 μl of 2× flourescence cell buffer: (1 ml 1M tris hcl ph: 7.5, 500 μl 1M KCL, 200 μl 1M NaCl2, 200 μl 0.1M MgCL2, 50 μl % 0.2 tween 20, 75 μl digitonin 20 mg/ml in DMSO ChemCruz cat no: sc-280675, 10 μl flourescence substrates including Suc-LLVY-AMC, Z-LLE-AMC, and Ac-RLR-AMC Ac-RLR-AMC, 8.1 ml H2O). After incubation at room temperature for a 5 minutes the flourescence of each sample were measured in a flourescence spectrometer (Biotek instrument-Synergy HT).

### Preparation of Cells Expressing the GFP-p21 Reporter.

For this purpose Retro-XTet-On Advanced Inducible Expression System (Clontech Cat no: 632104) was used.

Preparation of GFP-p21 cDNA.

pBSK-clo2D vector construct, which was previously prepared the cloning sites of the pBSK (−) vector as KpnI, ApaI, XhoI, SalI, PmeI, BamHI EcoRV, EcoRI, XbaI, NotI, HindIII, MfeI, EcoRI, PstI, SmaI, BamHI, XbaI, BstXI, and SacI in order to facilitate general DNA cloning was used to fuse GFP and p21 cDNAs. The GFP open-reading frame was obtained from pEF-GFP (pEF-GFP was a gift from Connie Cepko (Addgene plasmid #11154) by PCR using primer sequences (GFP-F ATCCACCGGTCGCCACCA TG and CTTGTACAGCTCGTCCATGC) and cloned into pBSK-clo2D which is cut by EcoRV restriction enzyme (SibEnzyme) and blunt ended by Phusion High-Fiderlity DNA Polymerase (Thermo Scientific). pBSK-clo2D-GFP vector was linearezed by cutting with EcoRV than dephosphorylated with Alkaline Phosphatase (Fermentas). P21 cDNA preparated and cloned as described below. For this purpose, RNA was isolated from HepG cells using RNase minikit (Qiagen). cDNA was generated by reverse transcription of 1 mg RNA obtained from the HepG cell, using random primers (Invitrogen) and M-MLV reverse transcriptase (Invitrogen). The p21 transcript was PCR amplified from the cDNA using the sense primer 5′-CAT GTC AGA AAC CGGCTG GGG-3′ and the anti-sense primer 5′-TTA GGG CTT CCT CTT GGA GA-3′.

P21 cDNA was then cloned into the phosphorylated vector using T4 DNA Ligase (Thermo Scientific). The fusing of the GFP and p21 was designed to remain in the codon frame. P21-GFP fused DNA fragment was obtained from the pBSK-clo2D vector by cutting with BamHI. pRetroX-Tight-Pur Vector was cut by BamHI restriction enzyme (Thermo Scientific) and dephospharylated and ligated with p21-GFP DNA. After the orientation construct was determined, pRetroX-Tight-Pur-GFP-p21 was reproduced in large volumes using DH5α E. coli. The Retro-X Tet-On Advanced Inducible Expression System requires the simultaneous presence of two retroviral constructs in a cell, Tet dependent rtTA transactivator (pRetroX-Tet-On-Advanced) and the target construct driven by the transactivator (pRetroX-Tight-Pur plus target gene). Thus, we first established HepG2, Hep3B, T98G cells integrating rtTA transgene. For this purpose, Hek293T cells were transfected by the pRetroX-Tet-On-Advanced and helper vectors pCMV-VSV-G, pUMVC, using CalPhos Mammalian Transfection Kit (631312-Takara bio). After 48 h supernatants containing rtTA virus were collected and target cells were infected under the presence of 10 mg/ml polybrene (TR-1001, Sigma-Aldrich). After 24 hours, the media of the cells were replaced with media containing neomycin (G418-RO, Sigma-Aldrich) 0.2-1 mg/ml and followed 10 to 15 days for selection. Then living cells were combined in a pool. Subsequently, similarly as the first infection with viruses containing pRetroX-Tight-Pur-GFP-p21 cDNA or pRetroX-TightPur control were obtained. The previously established rtTA expressing HepG2, Hep3B and T98G cells were infected with these viruses, and infected clones were selected by 0.5-3 mg/ml puromycin (P9620-Sigma-Aldrich) for 6 days. Infected clones were maintained in the presence of 250 ng/ml puromycin. The expression of GFP-p21 was followed by western blot and fluorescence microscope (Bio-Rad ZOE, U.S.A.) 12-24 hours after the adding doxacilline.

### Overexpression of USP14 Using the Retro-X Tet-on Advanced Inducible Expression System

The USP14 transcript was PCR amplified from the cDNA obtained from the HepG cell using the sense primer 5′-GCC GCC ACC ATG CCG CTC TAC TCC-3′ and the anti-sense primer 5′-TTA CTG TTC ACT TTC CTC TTC-3′. Usp14 cDNA was cloned into pBSK-clo2D which is cut by EcoRV restriction enzyme (SibEnzyme, E059). Usp14 was obtained from the pBSK-clo2D vector by cutting with BamHI. pRetroX-Tight-Pur retroviral vector was cut by BamHI restriction enzyme and dephospharylated and ligated with Usp14 cDNA with compatible cohasive end. After the orientation construct was determined, pRetroX-Tight-Pur-Usp14 was reproduced in large volumes using DH5α E. Coli.

Subsequently, similarly as described above, viruses containing pRetroX-Tight-Pur-Usp14 cDNA or pRetroX-TightPur control were obtained. The previously established rtTA expressing HepG2, Hep3B and T98G cells were infected with these viruses, and infected clones were selected by 0.5-3 mg/ml puromycin (P9620-Sigma-Aldrich) for 6 days. Infected clones were maintained in the presence of 250 ng/ml puromycin. The expression of Usp14 was followed by western blot.

### Preparation of Cells Stably Expressing the Ub-R-YFP.

The Ub-R-YFP open-reading frame was obtained from Ub-R-YFP was a gift from Nico Dantuma (Addgene plasmid #11948). Ub-R-YFP sequence was obtained from the original vector by cutting the plasmid with NheI and NotI enzymes (Thermo Scientific), blunt ended and was cloned into pBSK-clo2D which is cut by EcoRV restriction enzyme (SibEnzyme, E059). Ub-R-YFP was obtained from the pBSK-clo2D vector by cutting with BamHI. pLV-EF1a-IRES-Puro (85132-Addgene) vector was cut by BamHI restriction enzyme and dephospharylated and ligated with Ub-R-YFP. After the orientation construct was determined, pLV-EF1a-IRES-Puro-Ub-R-YFP was reproduced in large volumes using DH5a E. Coli.

Hek293T cells were transfected with the pLV-EF1a-IRES-Puro-Ub-R-YFP and helper vectors psPAX2 and pMD2.G using Calcium Phosphate Mammalian Transfection Kit (631312-Takara bio). After 48 h supernatants containing Ub-R-YFP lentivirus were collected and target cells were infected under the presence of 10 mg/ml polybrene. After 24 hours, the media of the cells were replaced with media containing 0.5-3 mg/ml puromycin for 6 days. Then living cells were combined in a pool. Infected clones were maintained in the presence of 250 ng/ml puromycin.

### Preparation of Cells Stably Expressing the LC3B.

Construction of pLV-EF1a-IRES-Puro-LC3B Vector Plasmid.

The LC3B open-reading frame was obtained from pEF-GFP (22418 pBABE-puro mCherry-EGFP-LC3B 22418-Addgene) by PCR using the sense primer 5′ TCG CCA CCA TGG TGA GCA AG and the anti-sense primer 5′ GAC TTA CAC TGA CAA TTT CA and cloned into pBSK-clo2D which is cut by EcoRV restriction enzyme (SibEnzyme, E059). LC3B was obtained from the pBSK-clo2D vector by cutting with BamHI. pLV-EFla-IRES-Puro (85132-Addgene) vector was cut by BamHI restriction enzyme and dephospharylated and ligated with LC3B. After the orientation construct was determined, pLV-EF1a-IRES-Puro-LC3B was reproduced in large volumes using DH5a E. Coli.

Hek293T cells were transfected with the pLV-EF1a-IRES-Puro-LC3B and helper vectors psPAX2 and pMD2.G using Calcium Phosphate Mammalian Transfection Kit (631312-Takara bio). After 48 h supernatants containing LC3B lentivirus were collected and target cells were infected under the presence of 10 mg/ml polybrene. After 24 hours, the media of the cells were replaced with media containing 0.5-3 mg/ml puromycin for 6 days. Then living cells were combined in a pool. Infected clones were maintained in the presence of 250 ng/ml puromycin. The expression of LC3B was followed by flouresence microscope (Bio-Rad ZOE, U.S.A. and confocal microcope-Zeiss LSM-510,Jena, Germany).

Preparation of Cells Stably Expressing the APP695 Using pLV-EFla-IRES-Puro Vector

The APP695 open-reading frame was obtained from pc DNA FRT TO-APP695 was a gift from Aleksandra Radenovic (Addgene plasmid #114193) by cutting HindIII (Thermo Scientific) and EcoRV enzymes and cloned into pBSK-clo2D. Further cloning and cell expression protocols were followed as described above.

Preparation of Lentiviral Vectors of USP14, PSMD14, and Regγ shRNA

Oligonucleotides with the following sense and antisense sequences were used for the cloning of small hairpin RNA (shRNA)-encoding sequences in lentiviral vector.

USP14shRNA-F
GATCCCCGGCTCAGCTGTTTGCGTTGTTCAAGAGACAACGCAAACAGCTG
AGCCTTTTTGGAAA
USP14shRNA-R
AGCTTTTCCAAAAAGGCTCAGCTGTTTGCGTTGTCTCTTGAACAACGCAA
ACAGCTGAGCCGGG
REGshRNA-F
GATCCCCGTGAGGCAGAAGACTTGGTTTCAAGAGAACCAAGTCTTCTGCC
TCACTTTTTGGAAA
REGshRNA-R
AGCTTTTCCAAAAAGTGAGGCAGAAGACTTGGTTCTCTTGAAACCAAGTC
TTCTGCCTCACGGG
PSMD14shRNA-F
GATCCCCGTCTATATCTCTTCCCTGGTTCAAGAGACCAGGGAAGAGATAT
AGACTTTTTGGAAA
PSMD14shRNAR
AGCTTTTCCAAAAAGTCTATATCTCTTCCCTGGTCTCTTGAACCAGGGAA
GAGATATAGACGGG

The oligonucleotides above were annealed and subcloned into the BglII-HindIII site of pSUPER (VEC-PBS-0002, Oligoengine, U.S.A.). To construct pLVsiRNA against USP14, PSMD14 and Regγ the BamHI-SalI fragments of the corresponding pSUPER plasmid were subcloned into the BamHI-SalI site of pRDI292 which is backbone plasmid of pLV.PARP1 #5 (a gift from Didier Trono-Addgene plasmid #14548) and pLV.Usp14shRNA, pLV.PSMD14shRNA, and pLV.Regγ shRNA plasmid vectors were prepared.

Hek293T cells were transfected with the pLV.shUSP14, pLV.shPSMD14, and pLV.shRegγ and helper vectors psPAX2 and pMD2.G using Calcium Phosphate Mammalian Transfection Kit (631312-Takara bio). After 48 h supernatants containing lentivirus were collected and target cells were infected under the presence of 10 mg/ml polybrene. After 24 hours, the media of the cells were replaced with media containing 0.5-3 mg/ml puromycin for 6 days. Then living cells were combined in a pool. Infected clones were maintained in the presence of 250 ng/ml puromycin. Effects of shRNAs on each proteins were followed with western blot.

Preparation of Liquid Culture of C. elegans

C. elegans strains used in this study were gift from Dr. Deniz Aksoy, Faculty of Sciency-Trakya University-Turkey. Preparation of liquid culture of C. elegans was prepared according to the Stiernagle, T. Maintenance of C. elegans (Feb. 11, 2006), WormBook, http://www.wormbook.org. Animals were grown on nematode growth medium (NGM) (50 mM NaCl, 2% agar, 2.5% peptone, 5 mg/L cholesterol, 1.0 mM CaCl2), 1.0 mM MgSO4, 25 mM potassium phosphate, pH 6.0) seeded with Escherichia coli OP50 at 20° C. using standard methods.

### Synchronization and Sterilization of Worms

For this purpose, the method described by Sulston, J. & Hodgkin, J. was followed. (See, Methods. in: Wood, W. B. (ed.) The Nematode Caenorhabditis elegans. (1988) Cold Spring Harbor Laboratory, Cold Spring Harbor: 587-606.; Solis, G. M., Petrascheck, M. Measuring Caenorhabditis elegans Life Span in 96 Well Microtiter Plates. 2011, J. Vis. Exp. (49)) Two-3 days old worms with eggs growing in NGM were transferred to 50 ml flask using S-medium. After centrifugation, worms were treated with 20% Alkaline Hypochlorite Solution and the eggs were transferred to the S-medium after washing with S-medium.

Concentrated E. coli OP50 was used as a food source.

Animals were incubated for 2 days at 20° C. until the animals reach the L4 stage.

To sterilize the animals 300 μL of a 6 mM Fluorodeoxyuridine (FUDR) stock solution was added to 10 ml medium. During the monitoring of their life span, the worms were kept by shaking at a constant speed of 200 rpm. Locomotor activities of the worms were recorded and available.

### Mice

The experimental protocols were approved by the Ethics Committee of Ankara University, Faculty of Medicine, Medical Sciences Experimental Research and Application Center (Date: Dec. 11, 2019 and Decision number: 2019-22-189). Adult (12-mounts-old) C57BL/6 mice (5 for each group) were used. The mice were housed at the Experimental Animal Research Laboratory of Ankara University Faculty of Medicine. Food and water were given without restriction. The ambient temperature was kept at 21±2° C., and locomotor activity tests were performed at this temperature. The animals were housed in a room with a 12-hour light/dark cycle. The procedures in the study were performed in accordance with the Ankara University Animal Care Committee according to international guidelines. Memantine (Sigma-Aldrich) was administered orally at 3 mg/Kg/per day with drinking water of one-year-old mice. Tests related to locomotor activities of the mice were recorded and available.

### Immunostaining Analysis of Cells for LC3 Amount

HepG2 and SH-SY5Y cells stably expressing LC3 were treated with 2 hours with ketamin and then were fixed in 4% paraformaldehyde (Sigma-Aldrich) for 20 min, and then permeabilized with 1% Tween-20 (v/v) (Sigma-Aldrich). The cells were blocked in PBS containing 5% milk powder and then incubated in anti-LC3 antibody (Sigma Aldrich) for two hours at RT. After washing three times for 10 min with PBS including the cells were incubated with Texax Red conjugated anti-rabbit antibody (Invitrogen, USA) for 1 h at room temperature. After washing three times for 10 min with PBS-BSA, petri dishes in a 4-μL drop of PBS-based mounting medium containing 1 μg/mL Hoechst 33,342 (Invitrogen, USA) was added for DNA labelling. For negative control, PBS instead of primary antibody was used. All fluorescently tagged specimens were examined and imaged using a Zeiss LSM-880 Airyscan system (Zeiss, Germany). The amount of LC3 were compared across all sections using identical exposure conditions.

### Staining of Mice Tissues

Whole frozen tissues were sectioned with a microtome and fixed in 4% paraformaldehyde, Tissues were washed in PBS and the individual staining methods used in accordance with the manufacturer's recommendations. Total pixels from fluorescence in the images were quantified using Adobe Photoshop software CS2 (Adobe Systems), and normalized to a nuclear stain.

### Tests to Assess Motor Phenotype in Mice

### Kondziela's Inverted Screen Test (Inverted Screen Test)

Inverted screen test described firstly Kondizela for muscle strength (Kondizela, 1964). The mouse was put on a center of the rectangular mesh screen (52 cm×32 cm) and the screen was rotated 180° over 2 seconds. The screen was held steadily 40-50 cm above a sawdust surface. Holding time were recorded for a maximum of 5 minutes. The inverted screen was scored previously by Deacon (Falling between 1-10 sec=1 Falling between 11-25 seconds=2 Falling between 26-60 seconds=3 Falling between 61-90 seconds=4 Falling after 90 seconds=5) (Deacon, 2013). According to previous studies, parameters such as Total mobility time % (Gleitz et al, 2017), the latency of falling time, and four limbs griping time (Roemers et al, 2019) were analyzed.

### Weight Test

Weight test use to measure muscle strength (Deacon, 2013). The mouse was held by the middle of the tail gently. The mouse was allowed to hold the weight with its forelimbs. The experiment was recorded by camera for a maximum of 5 minutes. Total griping time was evaluated.

### Open Field Test

Open Field Test described firstly Hall for “emotionality” (Hall, 1934). Open Field test is used for anxiety-like behavior (ALB) and locomotor activity. We conducted the test in a hypethral box, which was made of 62×68 cm blueboard. The base of the box was divided into 20 equal rectangles. Animals had placed in the central region of Open Field Test. The first 5 min of the record was used for the analysis of behavioral parameters such as total distance traveled (horizontal locomotor activity), total rearing number (vertical locomotor activity), central zone time (ALB), Central zone Latency (the time of first return to the central region: ALB), central zone entrance (ALB), unsupported rearing (vertical locomotor activity without touch test wall and exploratory behavior, sensitive for ALB), central escape latency (first escape time from central zone: ALB) (Çalιşkan et al, 2019, Çalιşkan et al, 2020).

### Grip Strength Test

grip strength tests are highly specific, in that they attempt to measure a single, well-defined aspect of behaviour. Grip strength has traditionally been measured in one of three ways: by testing the ability of the mouse to remain clinging to an inverted or tilted surface such as a wire grid or a cage lid for a period of time, usually up to 1 minute; by testing the ability of the mouse to hang on a wire with its forepaws for a preset length of time or until its grip fails; or by making specific measurements of the force required to pull the mouse off a narrow bar that it is gripping.

### Statistical Analysis.

All of the data are expressed as the means f standard errors of the means. For all of the analyses, P values were obtained from Student's t-test (unpaired, two tailed) or Spearman rank-correlation tests. All of the graphs were generated using Microsoft Excel.

The data are based on the results of at least three independent experiments. The error bars show the standard deviations. P values less than 0.05 were considered significant.

### The Figures of the Drawing are for Illustration Purposes Only, not for Limitation.

FIG. 1 A-D shows that NMDAR blockers ketamin and memantine reduce the amount of cell cycle related, p21, p53, p27, and cdc25A proteins. FIG. 1A show the effects of ketamin and mementine on amount of p21, p53, p27 and cdc25A, with western blot in the HepG2 cell. FIG. 1B shows that the effect of ketamin on p21 reduction is time dependent. FIGS. 1C and D show that the effect of NMDAR blockers on p21 is not due to protein synthesis inhibition, with the result of the experiment using cycloheximide.

FIGS. 2 A and B shows that p21 protein decrease due to NMDAR blocker is not related to the Akt1 inactivation. FIG. 2A shows that NMDR blockers decrease both Akt1 Ser473 phosphorylation and p21 protein amount at the same time shown by western blot. FIG. 2B shows that the decrease of Akt1's phosphorylation has no clear effect on the reduction of p21 protein. In other words, the effects of NMDAR blockers on Akt1 Ser473 phosphorylation and p21 protein amount occur independently of each other.

FIG. 3A-D shows the effects of proteasome inhibitors on proteins including p21, p53 and pAkt1 which are reduced in the presence of NMDAR blockers. FIG. 3A shows that the p21 decrease caused by ketamin is inhibited by proteasome inhibitor, Mg132. FIG. 3B shows that the p21 decrease caused by memantin is inhibited by proteasome inhibitor, Mg132. FIG. 3C shows that the p53 decrease caused by ketamin is inhibited by proteasome inhibitor, Mg132. (Memantin results were not shown). FIG. 3D shows that the decrease in Akt1 phosphorylation caused by ketamin is not prevented by proteasome inhibitor, Mg132.

FIGS. 4A and B show that that the NMDAR blockers decrease the amount of pTau (A), and Serin Racemase (B) and this decrease is prevented by the addition of proteasome inhibitor, Mg132. (The results of the T98G, SH-SY5A cells and memantine were not shown)

FIG. 5A-C show that ketamin and memantine have no effects on any of the 3 subtypes of protesome activity including trypsin, chemotrypsin and caspase-like activity, when the cell lysate used. FIGS. 4A, B, and C shows the proteasome activity in cell lysates with ketamin and without ketamin after the incubation with fluorogenic substrates, trypsin, chymotrypsin and caspase-like activities, respectively.

FIG. 6A-D show the trypsin, chemotrypsin, and caspase-like activities of protesome in the whole HepG2 cell line in the presence of ketamin and control, respectively using “In-Cell Fluorogenic Proteasome Assay”. NMDAR blockers increased the activities of trypsin, chemotypsin, and caspase-like enzymes depending on the amount of the drugs (FIG. 6D) (the results of HepG-chemotrypsin-ketamin experiment was shown only).

FIG. 7A-D shows the protesome activity of whole cell using the Cell-Based Proteasome luciferase systems (Promega) in HepG2 cell treated with ketamin and control. FIG. 4A shows the chemotrypsin-proteasome activity in whole cell using the Cell-Based Proteasome luciferase systems (Promega). FIG. 4B shows the chemotrypsin-proteasome activity in cell lysates using the Cell-Based Proteasome luciferase systems (Promega). FIGS. 7C and D shows the Trypsin and caspase-like activities of protesome of whole cell using the Cell-Based Proteasome luciferase systems (Promega), respectively.

FIG. 8A-C show the trypsin, chemotrypsin, and caspase-like activities of protesome using “In-Cell Fluorogenic Proteasome Assay”, in the whole T98G cell line in the presence of ketamin and control, respectively.

FIG. 9A-C show the effects of drugs including ketamine, memantin, MK801, IU1, and Mg132 on the activities of trypsin, chemotrypsin, and caspase-like of protesome in HepG2 cell line. All protesome activity tests were completed with whole cell using the In-Cell Fluorogenic Proteasome Assay. FIG. A-C shows trypsin, chemotrypsin, and caspase-like activities of protesome in the HepG2 cell line in the presence of ketamine, memantin, MK801, IU1, and Mg132 respectively. Results are mean+SEM. * P<0.05 for ketamin and mementine results compared to controls.

FIG. 10A-C show the effects of drugs including ketamine, memantin, MK801, IU1, and Mg132 on the activities of trypsin, chemotrypsin, and caspase-like of protesome in T98G cell line. All protesome activity tests were completed with whole cell using the In-Cell Fluorogenic Proteasome Assay. FIG. A-C shows trypsin, chemotrypsin, and caspase-like activities of protesome in the HepG2 cell line in the presence of ketamine, memantin, MK801, IU1, and Mg132 respectively.

FIG. 11A-D shows the effect of ketamine on autophagic activity in HepG2 cell with different methods. FIG. 11A shows LC3 turnover from LC3-I to LC3-II by western blot. FIG. 11B shows SQSTM1/p62 amount in the presence of Lysosome inhibitors NL (NH4C1 and leupeptin). FIG. 11C the LC3 gene construct was stably inserted into the cell and after its continuous expression is achieved, the changes in the punctata in the presence of ketamin was shown by confocal microscopy. FIG. 11D shows the effects of ketamin on the degredation of p21 and p53 in the presence of proteasome inhibitor or lysosome inhibitors with western blot. It shows that there is no relationship between p21 and p53 degredation and autophagy.

FIG. 12. The effects of NMDAR blockers on the ubiqutination processes. FIG. 12A shows the effects of molecules, which are involved in different steps of the ubiqutination processes, on degredation of Ub-YFP expressed in Hep3B cell.

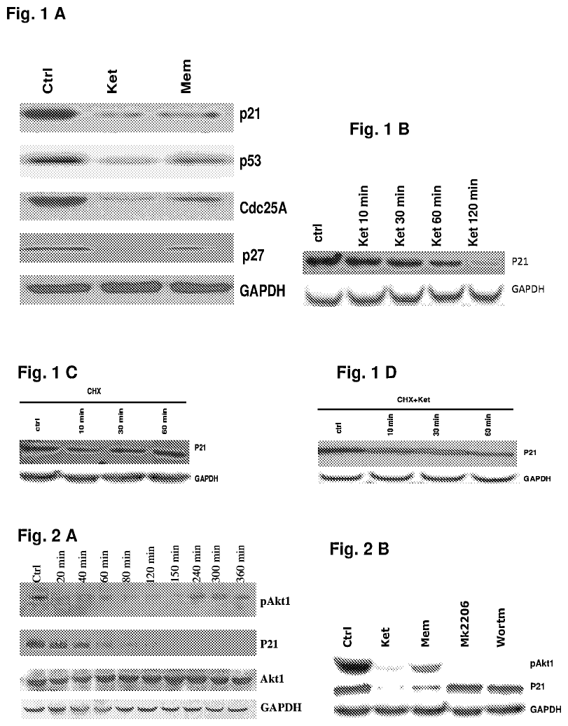

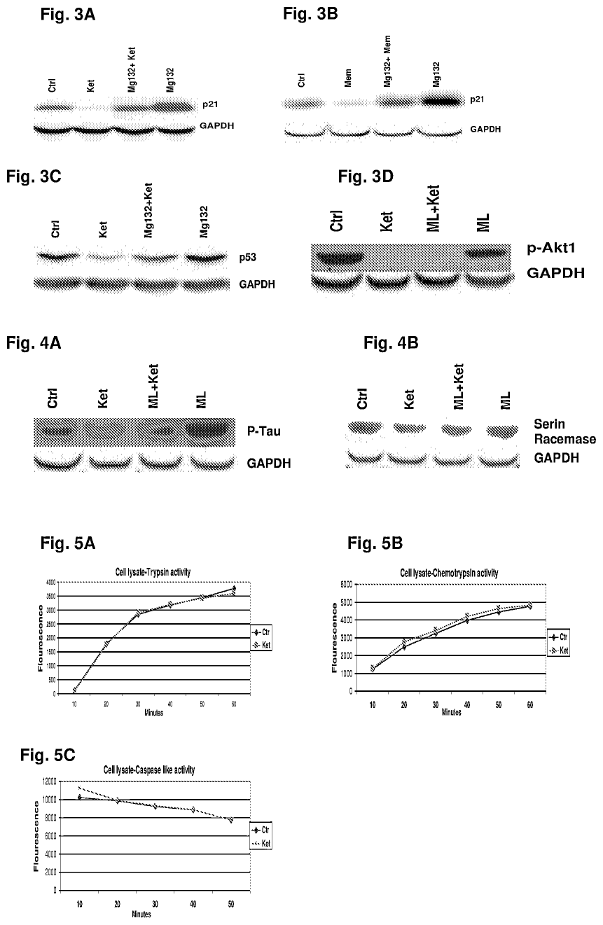

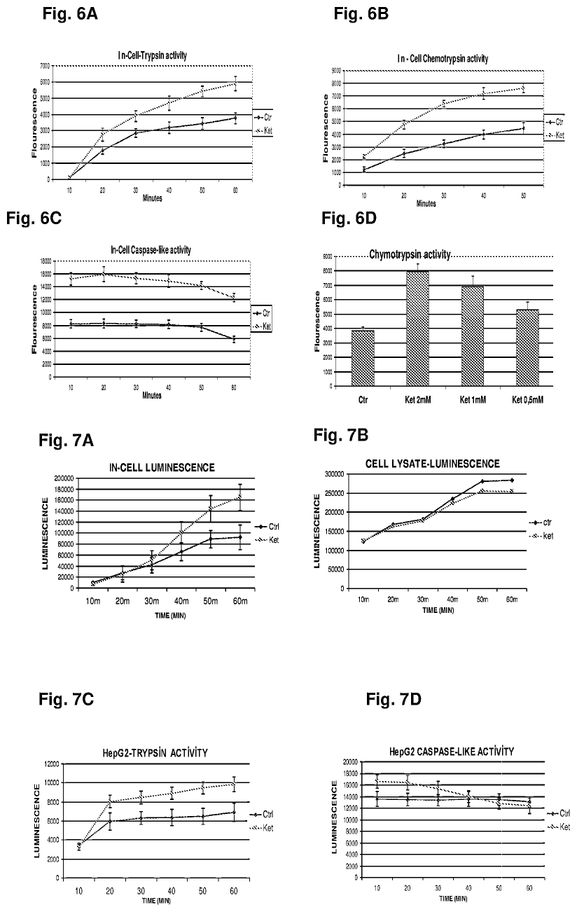

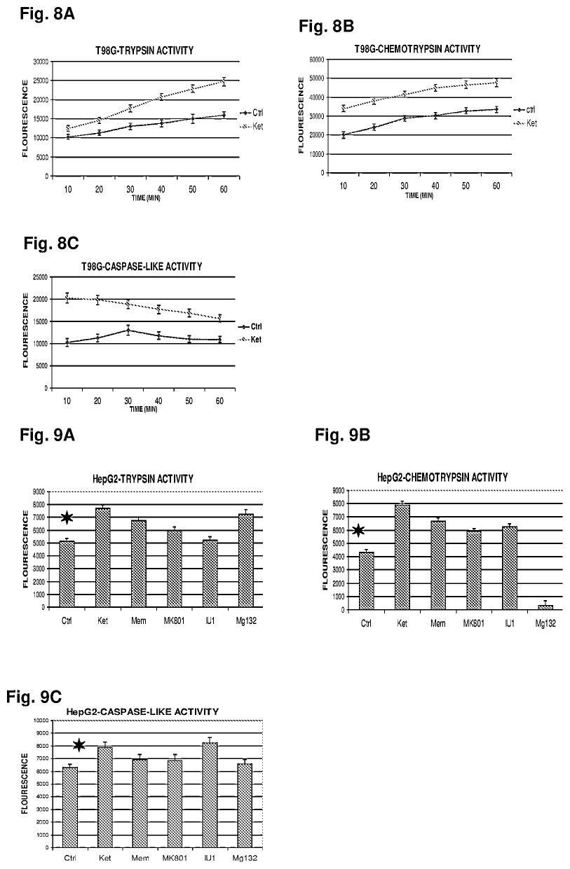

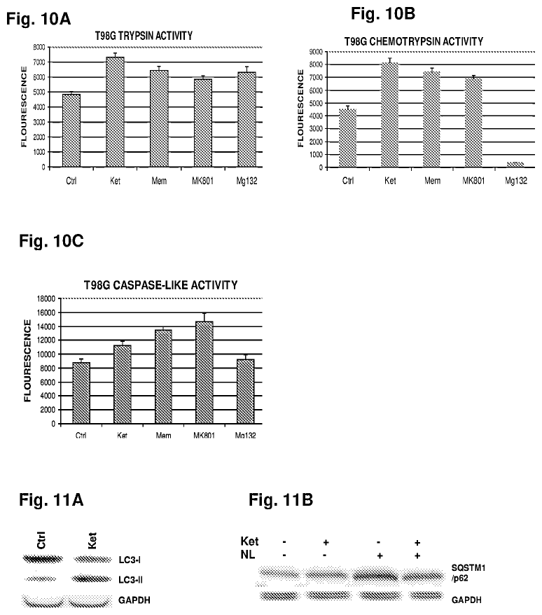

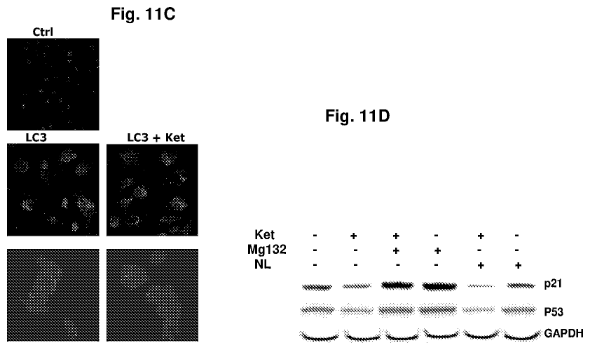

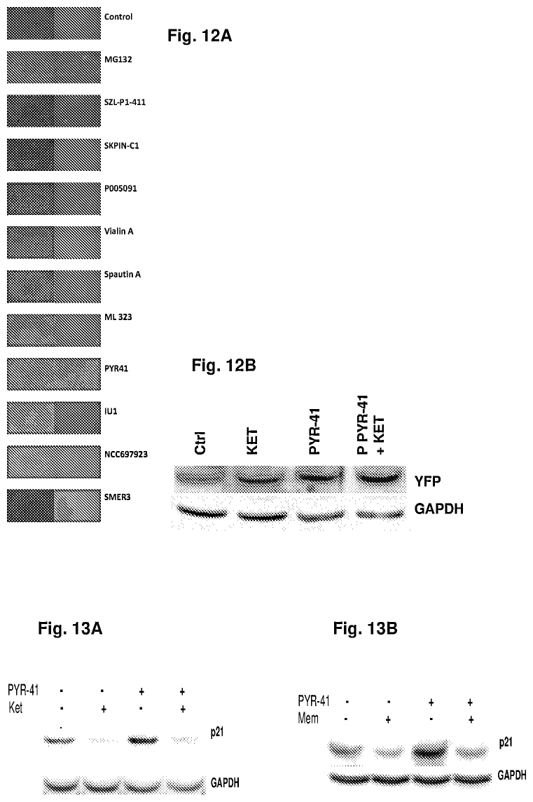

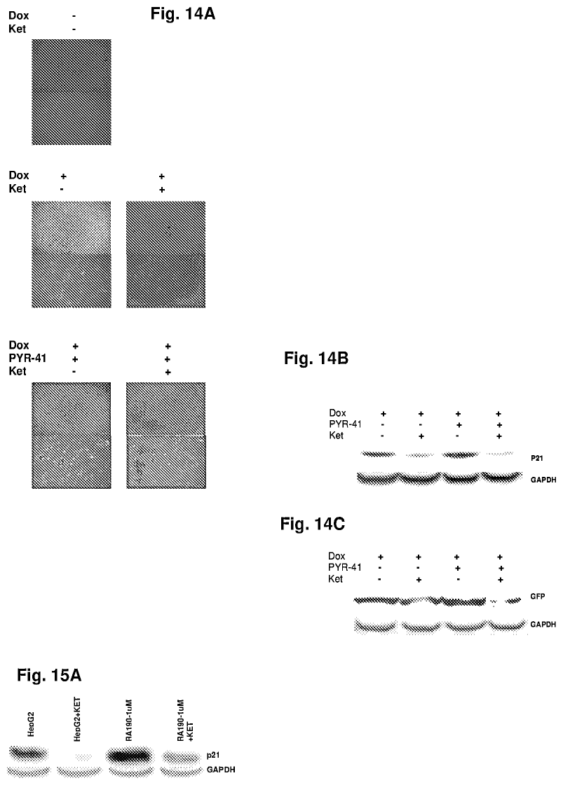

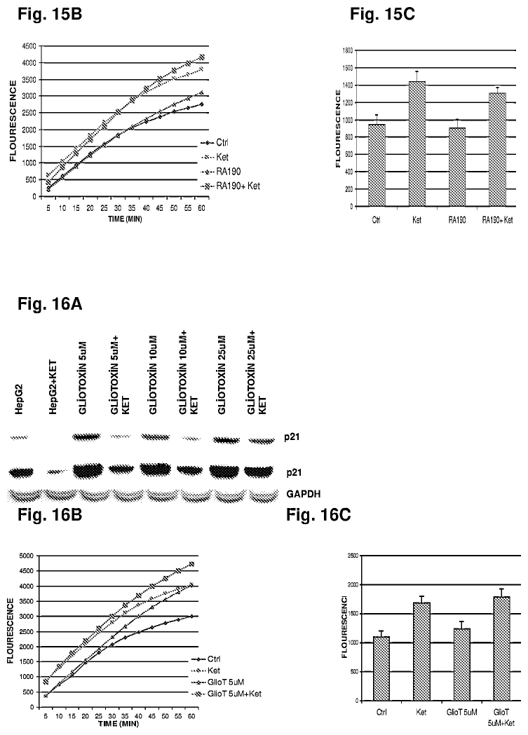

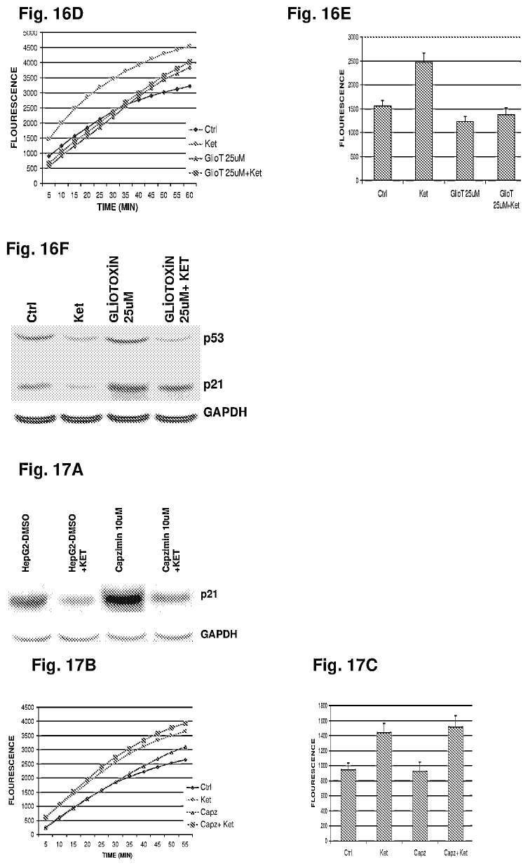

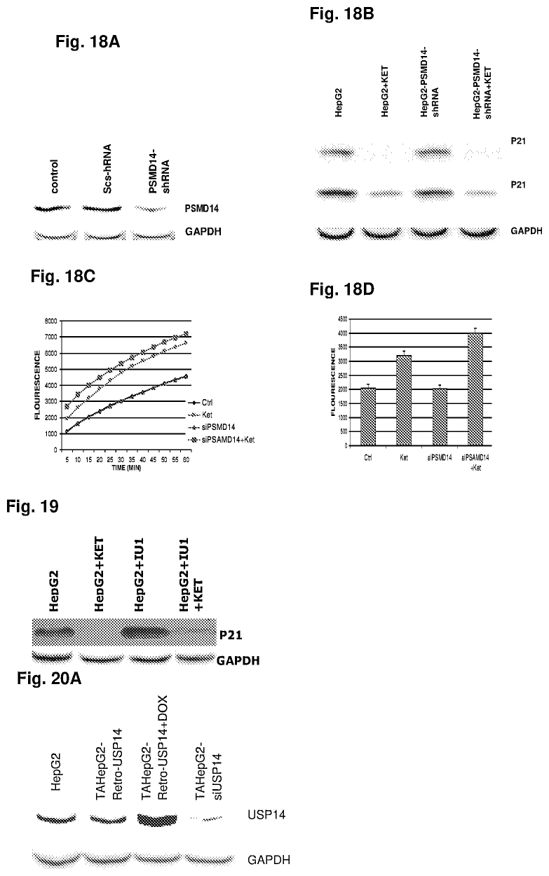

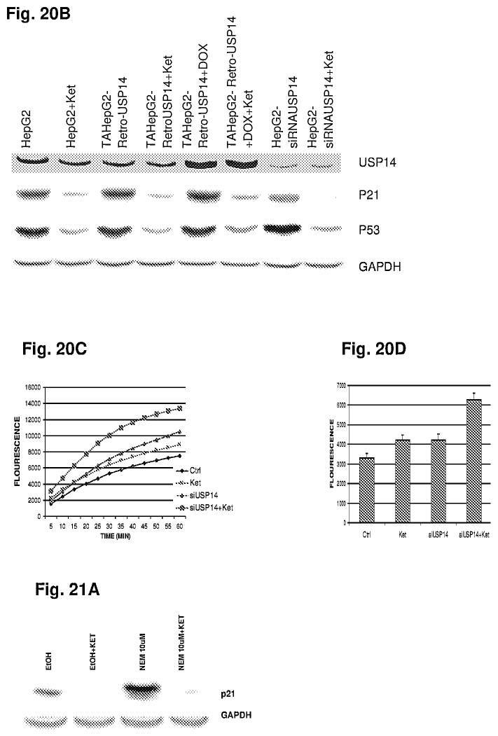

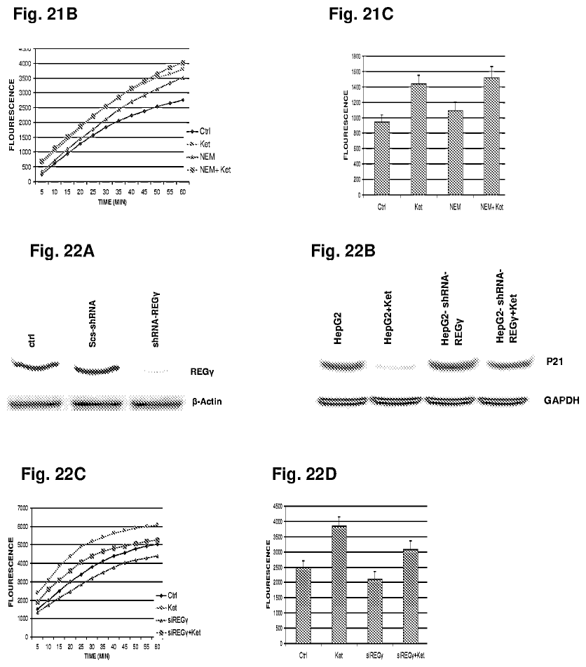

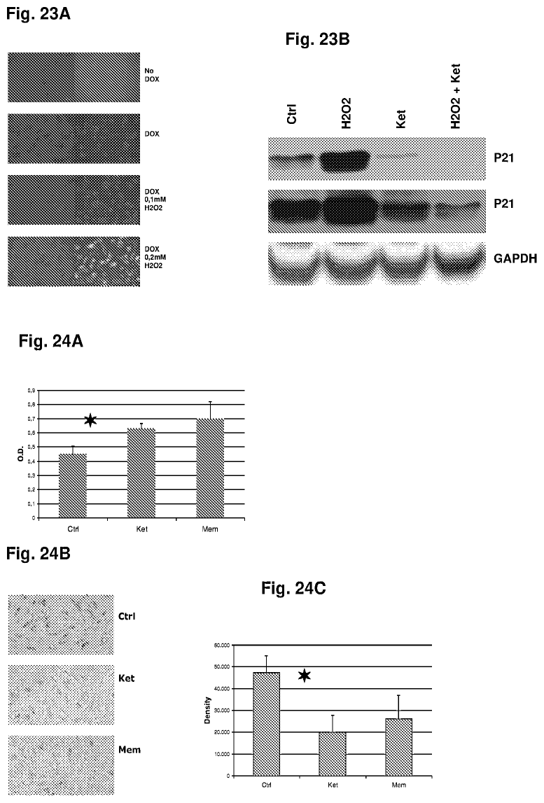

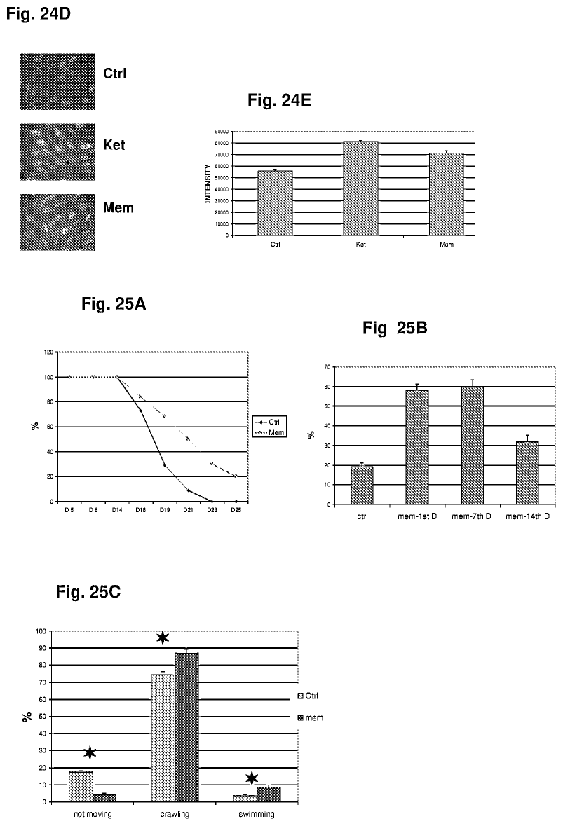

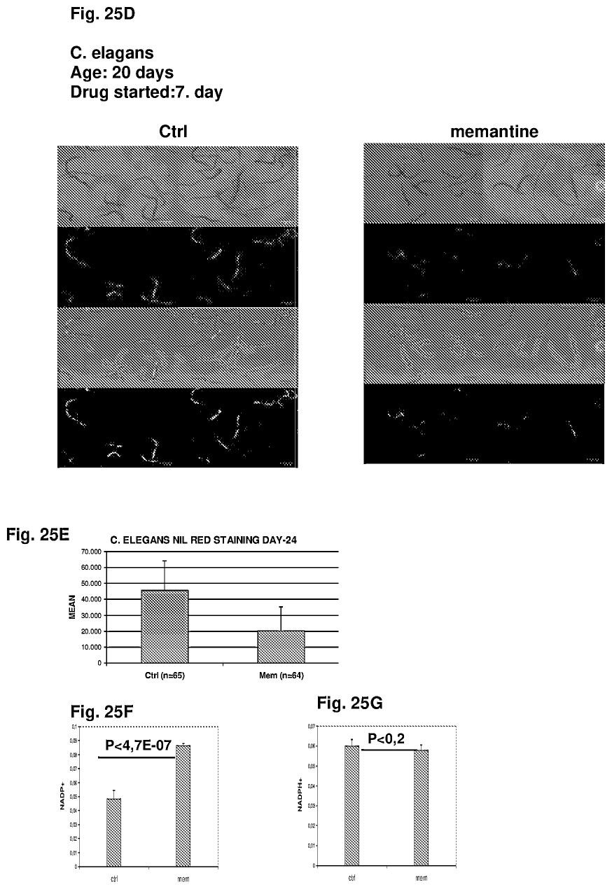

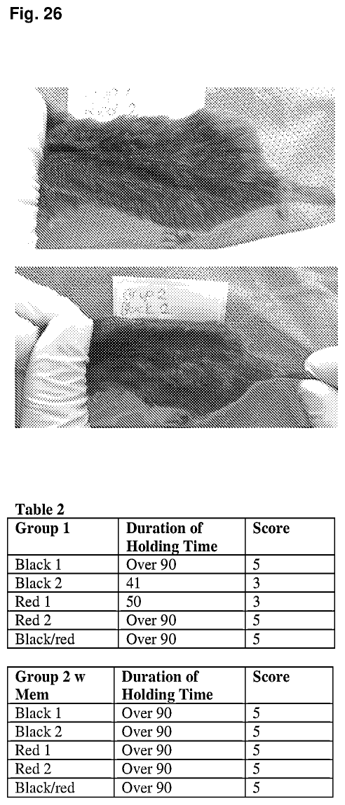

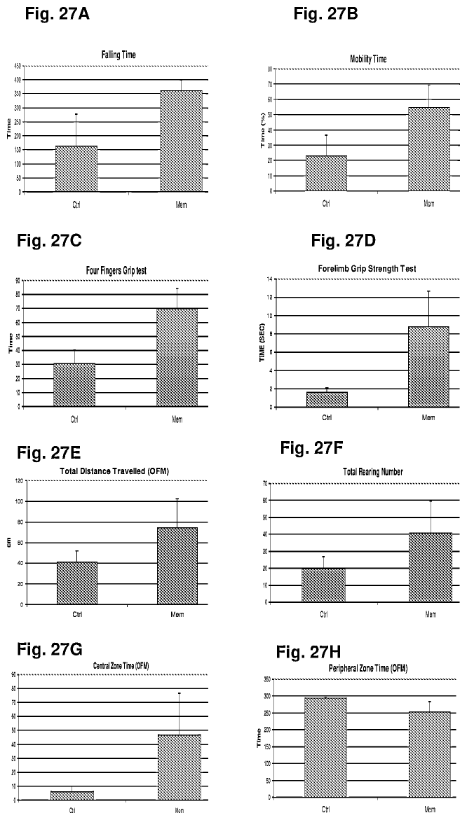

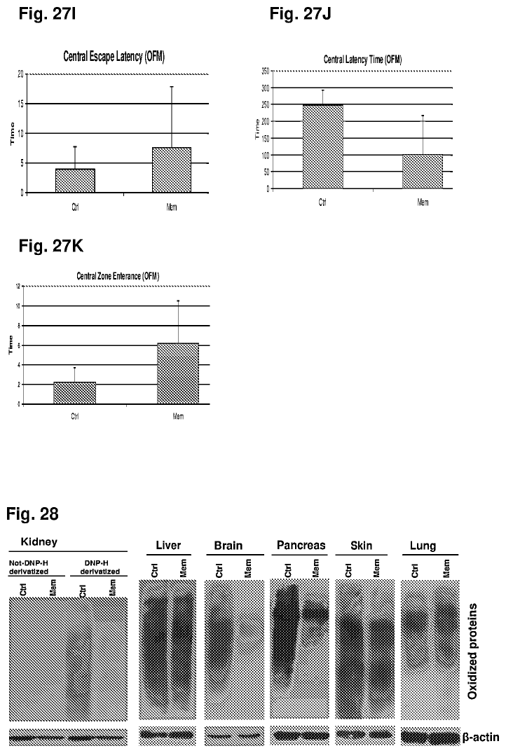

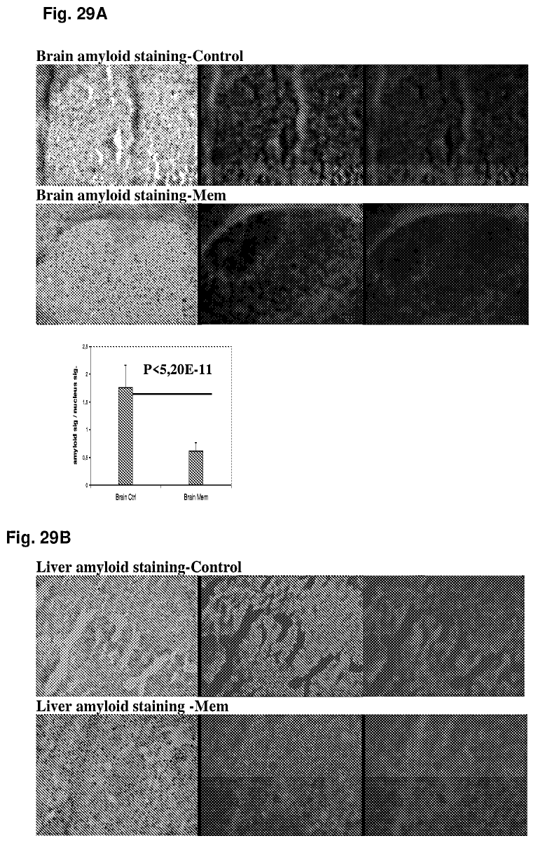

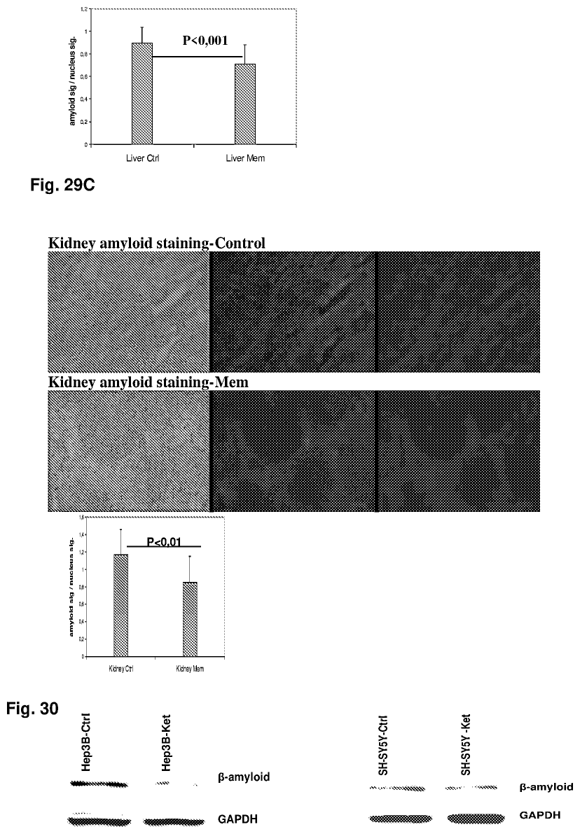

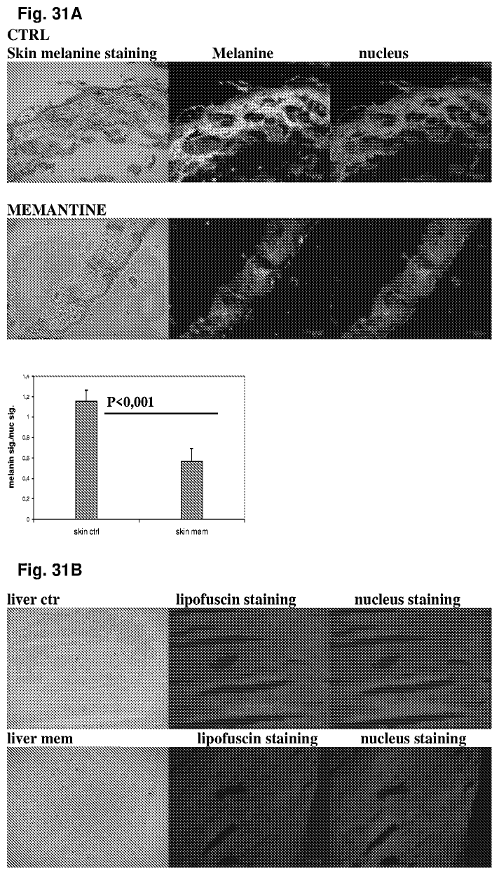

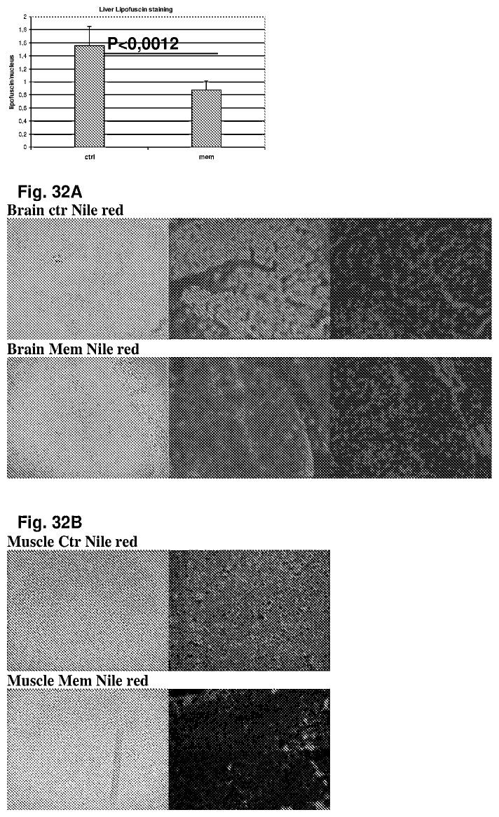

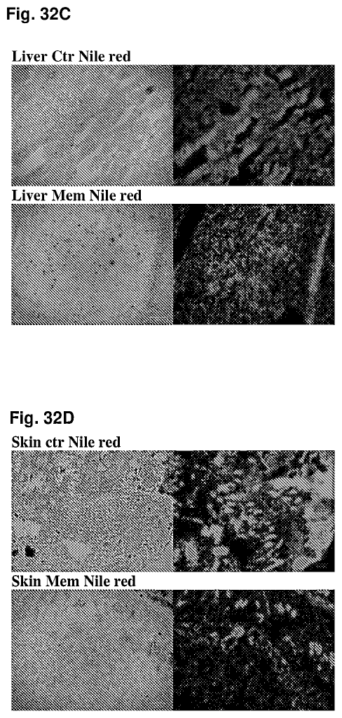

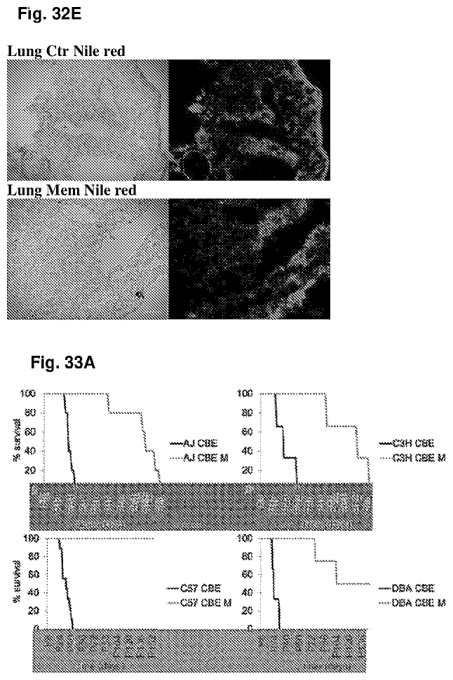

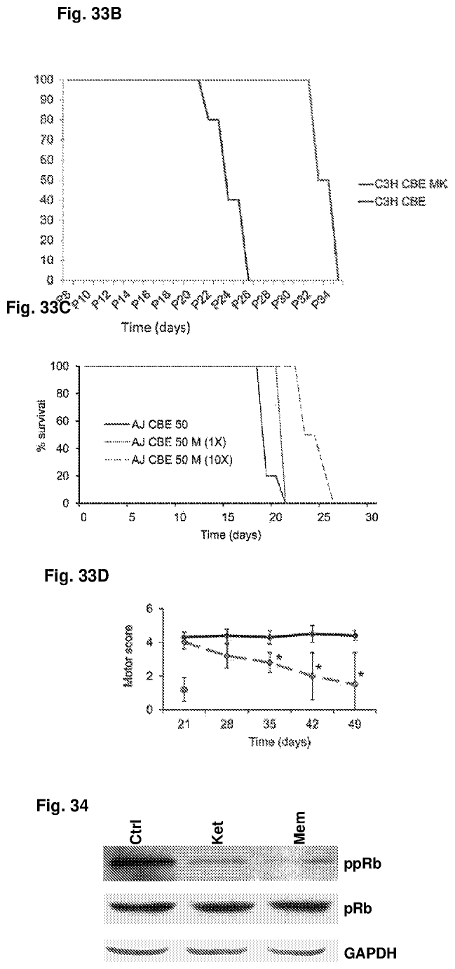

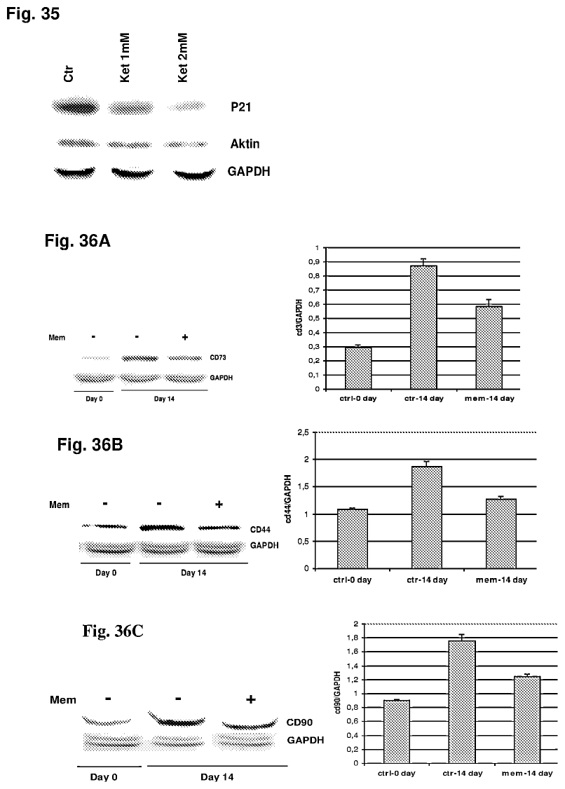

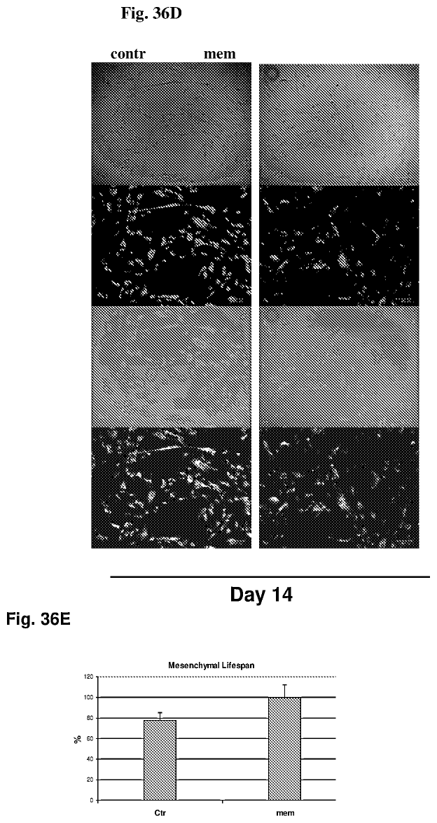

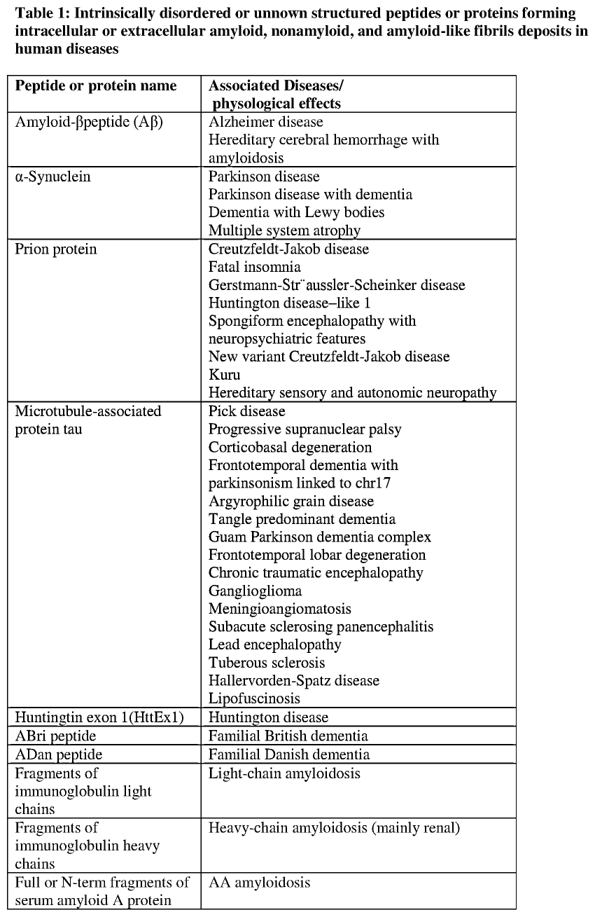

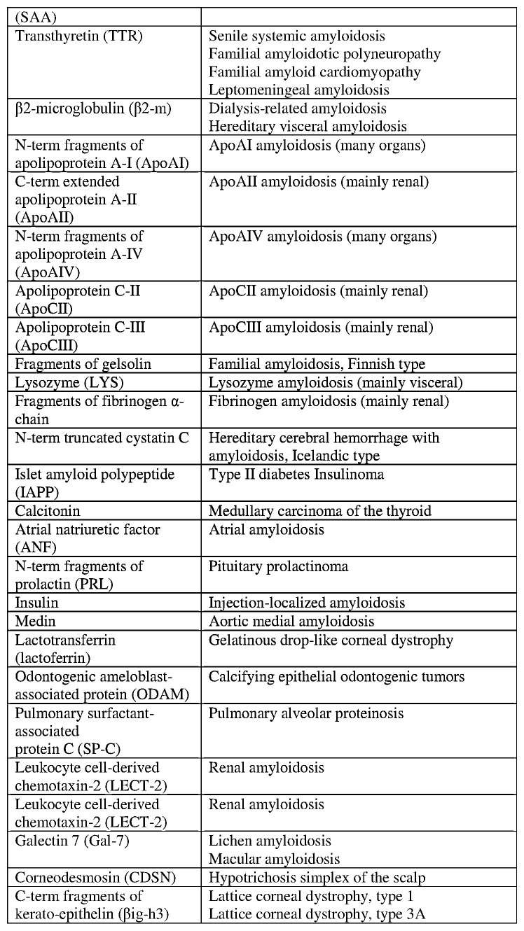

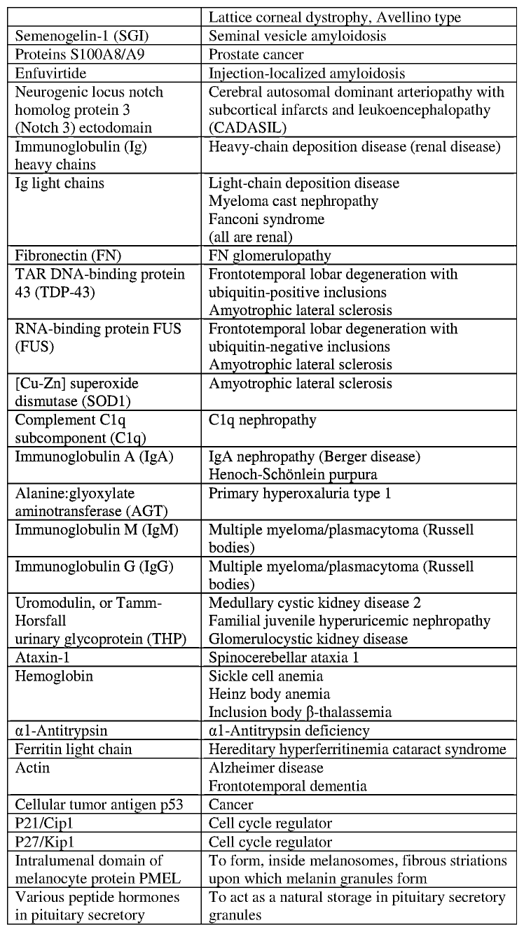

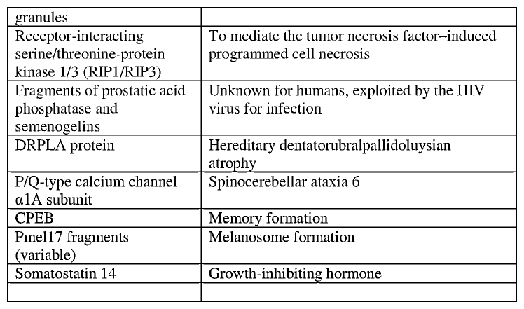

The molecules which are involved in different parts of the ubiqutination processes including PYR41 (Ubiquitin-activating enzyme (E1) inhibitor), NCC697923 (UBE2N E2 ubiquitin-conjugating enzyme inhibitör), SMER3 (ubiquitin ligase E3 inhibitor), SZL-P1-411 (Skp2 E3 ligase inhibitör), SKPIN C1 (Skp2 E3 ligase inhibitör), P005091 (USP7 inhibitor), Vialin A (USP4 and USP5 inhibitor), Spautin A (USP10 and USP13 inhibitor), ML 323 (USP1-UAF1 inhibitor), IU1 (USP14 inhibitor), and Mg132 (protesome inhibitor) were investigated. FIG. 12B shows that ketamin has no effect on Ub-YFP degredation. YFP protein amount was investigated by western blot in the presence of ketamin.

FIG. 13A,B shows that PYR41 increase endogenous p21 and ketamin or memantin effectively degreade p21 in the presence of PYR41 in HepG2 cells.

FIG. 14 shows that PYR41 increase GFP amount and ketamin decrease GFP amount in the Hep3B cells expressing of p21-GFP fusion protein. FIG. 14A shows that PYR41 increase GFP amount and ketamin decrease GFP amount in the Hep3B cells by flourescence microscope. FIG. 14B shows that PYR41 increase p21-GFP fusion protein amount and ketamin decrease p21-GFP fusion protein amount in the Hep3B cells by immunoblotting with anti-p21 antibody. FIG. 14c shows that PYR41 increase p21-GFP fusion protein amount and ketamin decrease p21-GFP fusion protein amount in the Hep3B cells by immunoblotting with anti-GFP antibody.

FIG. 15A-C show the effect of ketamin on p21 degradation and on the chemotrypsin activity of protesome in the presence and absence of RA190, selective inhibitor of the Rpn13. FIG. 12A shows the effect of ketamin on p21 degradation by western blot. FIG. B,C shows the effect of ketamin on chemotrypsin activity of protesome in the presence and absence of RA190 in HepG2 cell.

FIG. 16A-F show the effect of ketamin on p21 degradation and on the chemotrypsin activity of protesome in the presence and absence of Gliotoxin, selective inhibitor of the Rpn11 (psmd14). FIG. 16A shows the effect of different concentration of gliotoxin on p21 and the effect of ketamin on p21 in presence of gliotoxin in HepG2. FIGS. 16B and C show the effect of Gliotoxin in low (5 uM) concentrations on chemotrypsin activity of proteasome in the presence or absence of ketamin. FIGS. 16D and F show the effect of Gliotoxin in high (25 uM) concentrations on chemotrypsin activity of proteasome in the presence or absence of ketamin. FIG. 16F shows that ketamin affects the p21 and p53 degredation differently in the presence of high gliotoxin concentration in cell. Immunoblot is completed using p53 and p21 antibodies together on the same membrane (FIG. 16F)

FIG. 17A-C show the effect of ketamin on p21 degradation and on the chemotrypsin activity of protesome in the presence and absence of Capzimin, inhibitor of the Psmd14. FIG. 17A shows the effect of capzimin on p21 and the effect of ketamin on p21 in presence of capzimin in HepG2. FIG. 17B,C show the effect of ketamin on chemotrypsin activity of proteasome in the presence or absence of capzimin.

FIG. 18A-D show inhibition of Psmd14 expression by shRNA-producing lentiviral vector system and the effects of ketamin on p21 in psmd14 expression slienced cells. FIG. 18A shows inhibition of Psmd14 expression by shRNA-producing lentiviral vector system. Reducing levels of psmd14 were determined by western blot using anti-psmd-14 antibodies. GAPDH was used as a loading control. FIG. 18B shows the effects of ketamin on p21 in psmd14 expression slienced cells. FIG. 18C,D show the effect of ketamin on chemotrypsin activity of proteasome in the cell with Psmd14 expression inhibited

FIG. 19 shows the effect of ketamin on p21 degradation and on the chemotrypsin activity of protesome in the presence and absence of IU1 has previously been described to inhibit USP14.

FIG. 20A-D show the effect of ketamin on p21 degradation and on the chemotrypsin activity of protesome in the HepG cell in which USP14 is overexpressed or slienced. FIG. 20A shows inhibition of USP14 expression by shRNA-producing lentiviral vector system and overexpression of USP14 by the dox inducible retroviral vector system. Reduction or overexpression levels of USP14 were determined by western blot using anti-USP14 antibody. GAPDH was used as a loading control FIG. 20B shows the effects of ketamin on p21 in the USP14 overexpressing cells or in the USP14 expression slienced cells. FIGS. 20C and D show the effect of ketamin on chemotrypsin activity of proteasome in the USP14 overexpressing or USP14 expression slienced cells.

FIG. 21A-C show the effect of ketamin on p21 degradation and on the chemotrypsin activity of protesome in the presence and absence of N-Ethylmaleimide (NEM), general inhibitor of deubiqutination enzymes. FIG. 21A shows the effect of NEM on p21 and the effect of ketamin on p21 in presence of NEM in HepG2. FIG. 21B,C show the effect of ketamin on chemotrypsin activity of proteasome in the presence or absence of NEM.

FIG. 22A-D show inhibition of PA28 γ (REGgamma) expression by shRNA-producing lentiviral vector system and the effects of ketamin on p21 in the PA28γ expression slienced cells. FIG. 22A show inhibition of PA28 γ expression by shRNA-producing lentiviral vector system. The reduction levels of PA28 γ were determined by western blot using anti-PA28 γ antibodies. GAPDH was used as a loading control. FIG. 22B shows the effects of ketamin on p21 in the PA28 γ expression slienced cells. FIG. 22C,D show the effect of ketamin on chemotrypsin activity of proteasome in the PA28 γ expression slienced cells.

FIG. 23. A-B shows the effects of ketamin on endogenous p21 in the presence of H₂O₂ in TAHepG2-p21-GFP cell. FIG. 23A show the effect of H₂O₂ on the amount of p21-GFP fused protein depending on time and concentration. FIG. 23B shows the effects of ketamin on endogenous p21 in the presence of 0.2 mM H₂O₂.

FIG. 24A-F show the effects of NMDAR blockers on the cell viability and proliferation of human primary fibroblast. FIG. 24A shows results of MTT assay of cells with ketamin or memantine compared to the control in 24 hour period which is used to measure cellular metabolic activity as indicator of cell viability and proliferation in cells set up at passage 8 (*p<0.05). FIG. 24B, C shows the effect of ketamin and memantin on Senesence-SA β-gal activity in cells at passage 10. Human fibroblast cells were divided into two as control and NMDAR blockers added group at the 4th passage and followed under the same condution until the 10th passage. FIG. 24C shows quantification of SA β-gal stained cells. 70 cells from the each well marked by freehand selection after adjusting colour threshold colour to BOW, colour space to RGB and dark background and density measured using Imagej program and mean values of control, ketamin and memantine treated cells were compared (*p=0.0002 for ctrl-ket and *p=0.005 for ctrl-mem). FIG. 24D,E shows Lipid droplets stained by Nile Red in control fibroblast and fibroblast cells treated with ketamine or memantine at the passage 10. 70 cells from the each well marked by freehand selection after adjusting colour threshold colour to B&W, colour space to RGB and dark background and density measured using Imagej program and mean values of control, ketamin and memantine treated cells were compared (n=3 well of each, *p=0.0001 for ctrl-ket and *p=0.0002 for ctrl-mem).

FIG. 25A-D show the effects of memantin on life span, motility and fat metabolism of C. elegans. FIG. 25A shows that memantine increase life span of C. elegans (p=0.001) the mean lifespan values calculated by a log-rank (Kaplan-Meier) statistical test. FIG. 25B shows the effectiveness of the drug according to the onset of time. Memantin which was added to the medium on the 1st, 7th and 14th days and examined the mowing worms at the 20th day. FIG. 25C shows basic movements of C. elegans kept in liquid NGM with and without memantin. Follow-up of the recordings made on the 18th day showed that non-moving C. elegans were 3 times more in controls than those receiving memantine (n=3 plates from each of 2 different experiments, P=0.5 E-6). The number of C. elegans that can swim is 2,4 times higher in liquid NGM with memantine than in liquid NGM without memantine (P=0.0007). FIGS. 25D and E shows the amount of lipid amount of C. elegans grow in an environment containing memantin and an environment without memantine on the 20th day. Lipid amount was determined by Nile Red staining. Sixty C. elegans from the each groups marked by freehand selection after adjusting colour threshold colour to B&W, colour space to RGB and dark background and density measured using Image J software (http://rsbweb.nih.gov/ij/) and mean values of control, and memantine treated cells were compared and P-values were calculated using a two-tailed, two-independent sample Student's t-test (*P=03.2E-9 for ctrl-mem). FIGS. 25 F and G show the amount of NADP+ and NADPH+ in C. elagans grown in medium with memantine compared to the C. elegans grown in medium without memantine, on the 19th day, P<4.7E-07 and P<0.2 for NADP+ and NADPH+, respectively.

FIG. 26 shows the color change and condition of mice fur in 22 months of age received memantin and without memantine. The picture represents two groups.

FIG. 27A-K and table 2 show the effects of memantin on motor activities of mice. FIG. 27A shows that mice receiving memantine could stay in the inverted frame for twice as long (P=0.006) compared to control in the Kondziela's inverted screen test (holding time). FIG. 27B shows that mice receiving memantine were able to move more than twice as much as those who did not receive memantine (P=0.007) in the mobility time of the Kondziela's inverted screen test. FIG. 27C shows the four fingers grip test of mice. It was determined that group of mice that received memantine constantly used fingers and stayed on the wire for more than two times than the group that did not received memantine (P=0.001). FIG. 27D shows forelimb grip strength test of mice, in which the mice that received memantine could hold the weight with their for limbs for four times longer than those who did not receive memantine (P=0.003). FIG. 27E shows the mobility time of mice groups. It was determined by of the movements of mice in the OFM apparatus for five minute recordings. It was determined that mice receiving memantine walked nearly two times more than mice not receiving memantine (P=0.007). FIG. 27F shows the total rearing activity test which showed that mice receiving memantine had twice as much rearing activity than mice who did not receive memantine (P<0,05). FIG. 27G shows the Central zone time test which described as the time in which mice stay in the central zone of the OFM in 5 minute movements. it was determined that mice receiving memantine remained in the central region 4 times more than mice not receiving memantine (P=0.01).

FIG. 27H shows Peripheral Zone Time test results in which mice not receiving memantine remained in the peripheral regions more than mice receiving memantine (p=0.01). FIGS. 271J, and K show the results of the Central escape latency, Central latency time, and Central zone entrance studies, which are other studies of OFM. Although the means of all them differed at least twice in favor for the mice receiving memantin, the p value was determined to be lower than 0.05 in all three studies (P values 0.4, 0.07,and 0.08, respectively).

FIG. 28 shows the oxidation status of proteins in tissues of 24 month old mice receiving memantin for 12 months and control mice. The tissue proteins were converted to DNP-hydrazone, and then western blot was completed in accordance with the manufacturer's recommendations.

FIG. 29A-C show the effects of memantin on amyloid accumulation in mice tissues including brain, liver, and kidney, respectively. The tissues of the mice receiving and not receiving the memantine were examined at 22 months by amyloid staining and normalized to a nuclear stain in accordance with the manufacturer's recommendations.

FIG. 30 shows the effects of ketamin on beta-amyloid precursor protein in Hep3B and SH-SY5Y neuroblastome cells expressing APP695 (beta-amyloid precursor protein) with western blot.

FIGS. 31A and B show the effects of memantine on melanin (skin) and lipofuscin (liver) accumulation, respectfully in mice tissues. The tissues of the mice receiving and not receiving the memantine were examined at 22 months by melanin or lipofuscin staining and normalized to a nuclear stain in accordance with the manufacturer's recommendations.

FIG. 32A-E show the effects of memantin on lipid accumulation in liver, brain, muscle, lung, skin, pancreas, and kidney of mice with the presence of memantine and control mice groups at 22 months. The tissues of the mice were stained with Nile red and investigated.

FIG. 33 A-D are the figures which were adapted from the patent of Futerman et al. US 2170273917 A1 show that memantine extend the lifespan of neuropathic GD mice and increase the muscle strength. FIG. 33A shows Kaplan Meier survival curves of male mice from A/J (AJ), C3H/HeJ (C3H), DBA/2J (DBA), and C57BL6/JolaHsd (C57) strains treated daily with CBE (25 mg/kg day) (CBE, black lines) or CBE (25 mg/kg) plus memantine (3 mg/kg day) (CBE-M, gray lines) starting at P8. FIG. 33B shows that NMDAR blocker (MK-801) increase C3H mice survival. Male mice from the C3H/HeJ (C3H) strain were treated daily with CBE (25 mg/Kg day) (black line) or CBE (25 mg/Kg day) plus MK-801 (0.3 mg/Kg day) (gray line) starting at P8. FIG. 33C shows that increasing the memantine dose by 10 fold increases the life span of mice more.

FIG. 33D shows progression of motor coordination in A/J PBS-treated mice (black continuous line) (n=5), A/J CBE-treated mice (gray dot) (n=4) and A/CBE memantine (dotted line) (n=5), as assessed by the hanging wire test. Results are mean+SEM. * P<0.05 compared to PBS controls.

FIG. 34 show the effects of ketamin and memantine on Retinoblastoma (pRb) protein in HepG2 cell. NMDAR blockers did not cause a change in the amount of pRb protein, they caused a dramatic decrease in the amount of phospho-pRb (FIG. 34)

FIG. 35 show the effects of ketamin on beta-actin protein in HepG2 cell. Ketamin increase the degredation of beta-actin protein dose dependent (FIG. 35).

FIG. 36A-E show the effects of memantine on sternness characteristics of the Mesenchymal Stromal Cells. FIG. 36A-C show the protein expression amounts of the differentiation markers including cd44 (A), cd73 (B), and cd90 (C) in the stem cells, received or not memantin, harvested at day 14. FIG. 36D shows the amount of lipid droplet in the stem cells harvested at day 14 in control and memantin received cells. FIG. 36E show the lifespan of human mesenchymal stem cells with or without memantine. FIG. 36E shows the MTT test of the cells on the day 21 of passages (P<0.034).

### REFERENCES

Ali H, Al-Yatama M K, Abu-Farha M, Behbehani K, Al Madhoun A. Multi-lineage differentiation of human umbilical cord Wharton's Jelly Mesenchymal Stromal Cells mediates changes in the expression profile of sternness markers. PLoS One. 2015 Apr. 7; 10(4)
Ahmed H, Haider A and Ametamey S M. N-Methyl-D-Aspartate (NMDA) receptor modulators: a patent review (2015-present) Expert. Opin. Ther. Pat. 2020 October; 30(10):743-767
Aiken C T, Kaake R M, Wang X, and Huang L. Oxidative Stress-Mediated Regulation of Proteasome Complexes. Molecular & Cellular Proteomics 10:10.2011
Alzheimer's Association—https://www.azm.org
Ambros V. Maintenance of C. elegans. Theresa Stiernagle, Caenorhabditis Genetics Center, University of Minnesota, Minneapolis, MN 55455 USA. (Feb. 11, 2006), WormBook, ed.
Ariumi Y, Turelli P, Masutani M, Trono D. DNA damage sensors ATM, ATR, DNA-PKcs, and PARP-1 are dispensable for human immunodeficiency virus type 1 integration. J Virol. 2005 March 79(5):2973-8.
Auld et al. The assay Guidance Manual; https://www.ncbi.nlm.nih.gov/books/
Austad S. N. The Geroscience Hypothesis: Is It Possible to Change the Rate of Aging?. In: Sierra F., Kohanski R. (eds) (2016) Advances in Geroscience.
Balu D T, Pantazopoulos H, Huang CCY, Muszynski K, Harvey T L, Uno Y, Rorabaugh J M, Galloway C R, Botz-Zapp C, Berretta S, Weinshenker D, Coyle J T. Neurotoxic astrocytes express the d-serine synthesizing enzyme, serine racemase, in Alzheimer's disease. Neurobiol Dis. 2019 October; 130:104511
Blasiak J, Szczepanska J, Fila M, Pawlowska E, Kaarniranta K. Potential of Telomerase in Age-Related Macular Degeneration-Involvement of Senescence, DNA Damage Response and Autophagy and a Key Role of PGC-1alpha. Int J Mol Sci. 2021 Jul. 3; 22(13):7194.
Boselli M, Lee B H, Robert J, Prado M A, Min S W, Cheng C, Silva M C, Seong C, Elsasser S, Hatle K M, Gahman T C, Gygi S P, Haggarty S J, Gan L, King R W, Finley D. An inhibitor of the proteasomal deubiquitinating enzyme USP14 induces tau elimination in cultured neurons. J Biol Chem. 2017 Nov. 24; 292(47):19209-19225.
Brehm A and Krüger E Dysfunction in protein clearance by the proteasome: impact on autoinflammatory diseases. Semin Immunopathol (2015) 37:323-333
Breydo L, Redington J M, Uversky V N Effects of Intrinsic and Extrinsic Factors on Aggregation of Physiologically Important Intrinsically Disordered Proteins. International Review of Cell and Molecular Biology, Volume 329 #2017 Elsevier Inc. ISSN 1937-6448
Brown T A Genomes. 2nd edition. Oxford, Wiley-Liss: 2002
Bussian T J, Aziz A, Meyer C F, Swenson B L, van Deursen J M, and Baker D J Clearance of senescent glial cells prevents tau-dependent pathology and cognitive decline. Nature. 2018 October; 562(7728): 578-582.
Caliskan H, Akat F, Tatar Y, Zaloglu N, Dursun A D, Bastug M, Ficicilar H. Effects of exercise training on anxiety in diabetic rats. Behav Brain Res. 2019 Dec. 30; 376:112084.
Caliskan H, Akat F, Omercioglu G, Bastug G, Ficicilar H, Bastug M. Aerobic exercise has an anxiolytic effect on streptozotocin induced diabetic rats. Acta Neurobiol Exp (Wars). 2020; 80(3):245-255.
Can A, Ulus A T, Cinar O, Topal Celikkan F, Simsek E, Akyol M, Canpolat U, Erturk M, Kara F, Ilhan O Human Umbilical Cord Mesenchymal Stromal Cell Transplantation in Myocardial Ischemia (HUC-HEART Trial). A Study Protocol of a Phase 1/2, Controlled and Randomized Trial in Combination with Coronary Artery Bypass Grafting. Stem Cell Rev Rep. 2015 October; 11(5):752-60.
Cary D C, Peterlin B M. Proteasomal Inhibition Potentiates Latent HIV Reactivation. AIDS Res Hum Retroviruses. 2020 October; 36(10):800-807
Cauli O, Gonzilez-Usano A, Cabrera-Pastor A, Gimenez-Garzó C, López-Larrubia P, Ruiz-Sauri A, Hernindez-Rabaza V, Duszczyk M, Malek M, Lazarewicz J W, Carratalá A, Urios A, Miguel A, Torregrosa I, Carda C, Montoliu C, Felipo V. Blocking NMDA receptors delays death in rats with acute liver failure by dual protective mechanisms in kidney and brain. Neuromolecular Med. 2014 June; 16(2):360-75.
Chiti F and Dobson C M Protein Misfolding, Amyloid Formation, and Human Disease: A Summary of Progress Over the Last Decade. Annu. Rev. Biochem. 2017.86:27-68.
Chondrogianni N, Voutetakis K, Kapetanou M, Delitsikoua V, Papaevgenioua N, Sakellari M, Lefaki M, Filippopoulou K, Gonos E. Proteasome activation: An innovative promising approach for delaying aging and retarding age-related diseases. Ageing Research Reviews 23 (2015) 37-55
Collins G A and Goldberg A L. The Logic of the 26S Proteasome. Cell. 2017 May 18; 169(5): 792-806.
Cull-Candy S, Brickley S and Farrant M NMDA receptor subunits: diversity, development and disease. Curr Opin Neurobiol 2001 June; 11(3):327-35.
Davies K J A Degradation of oxidized proteins by the 20S proteasome. Biochimie 83 (2001) 301-310
Dikic I Proteasomal and Autophagic Degradation Systems. Annu. Rev. Biochem. 2017. 86:193-224
Ding W X, Ni H M, Gao W, Yoshimori T, Stolz D B, Ron D, and Yin X M. Linking of Autophagy to Ubiquitin-Proteasome System Is Important for the Regulation of Endoplasmic Reticulum Stress and Cell Viability. AJP August 2007, Vol. 171, No. 2
Das J Repurposing of Drugs—The Ketamine Story. J. Med. Chem. 2020 Nov. 25; 63(22):13514-13525
Dobson C M The Amyloid Phenomenon and Its Links with Human Disease. Annu. Rev. Biochem. 2017.86:27-68.
Du J, Li X H, and Li Y J Glutamate in peripheral organs: Biology and pharmacology. European Journal of Pharmacology 784 (2016) 42-48
Eltzschig H K, Eckle T Ischemia and reperfusion-from mechanism to translation. Nat Med (2011) 17: 1391-1401).
Feldmann L K, Prieult F, Felzen V, Thal S C, Engelhard K, Behl C, and Mittmann T Proteasome and Autophagy-Mediated Impairment of Late Long-Term Potentiation (l-LTP) after Traumatic Brain Injury in the Somatosensory Cortex of Mice. Int. J. Mol. Sci. 2019, 20, 3048
Fernández-Albarral J A, de Julián-López E, Soler-Dominguez C, de Hoz R, López-Cuenca I, Salobrar-Garcia E, Ramirez J M, Pinazo-Durán M D, Salazar J J, The Role of Autophagy in Eye Diseases. Ramirez AI. Life (Basel). 2021 Feb. 27; 11(3):189.
Fleckenstein M, Keenan T D L, Guymer R H, Chakravarthy U, Schmitz-Valckenberg S, Klaver C C, Wong W T, Chew E Y.. Age-related macular degeneration. Nat Rev Dis Primers 2021 May 6; 7(1):31.
Futerman et al. U.S. Pat. Nos US 2017/0273917 A1 and date Sep. 28, 2017 (Patent)
Gao Z, Gammoh N, Wong P M, Bromage H E, Tempst P and Jiang X Processing of autophagic protein LC3 by the 20S proteasome. Autophagy 6:1, 126-137; Jan. 1, 2010;
Gan J, Leestemaker Y, Sapmaz A and Ovaa H Highlighting the Proteasome: Using Fluorescence to Visualize Proteasome Activity and Distribution. Frontiers in Molecular Biosciences 2019 Mar. 22; 6:14
Giordano F J Oxygen, oxidative stress, hypoxia, and heart failure. J Clin Invest (2005) 115: 500-508)
Glass, C. K., Saijo, K., Winner, B., Marchetto, M. C.& Gage, F. H. Mechanisms underlying Inflammation in neurodegeneration. Cell 140, 918-934 (2010).
González-Garza M T, Cruz-Vega D E, Cardenas-Lopez A, de la Rosa R M, Moreno-Cuevas J E. Comparing stemness gene expression between stem cell subpopulations from peripheral blood and adipose tissue. Am J Stem Cells. 2018 Jun. 1; 7(2):38-47.
Groc L, Bard L, Choquet D., Surface trafficking of N-methyl-D-aspartate receptors: physiological and pathological perspectives. Neuroscience. 2009 Jan. 12; 158(1):4-18.
Grune, T. Oxidative Stress, Aging and the Proteasomal System. Biogerontology 2000, 1(1), 31-40.
Halminen O, Vesikansa A, Mehtälä J, Hörhammer L, Mikkola T, J Virta L J, Ylisaukko-Oja T, Linna M Early Start of Anti-Dementia Medication Delays Transition to 24-Hour Care in Alzheimer's Disease Patients: A Finnish Nationwide Cohort Study. J Alzheimers Dis. 2021 Apr. 3.
Hartl F U. Protein misfolding diseases. Annu Rev Biochem. 2017 Jun. 20; 86:21-26
Hayer A, Shao L, Chung M, Joubert L M, Yang H W, Tsai F C, Bisaria A, Betzig E, Meyer T. Engulfed cadherin fingers are polarized junctional structures between collectively migrating endothelial cells. Nat Cell Biol. 2016 December; 18(12):1311-1323.
Hipp M S, Sae-Hun Park S H and Hartl F U Proteostasis impairment in protein misfolding and -aggregation diseases Trends in Cell Biology, September 2014, Vol. 24, No. 9
Hirsch C and Ploegh H L Intracellular targeting of the proteasome. Trends Cell Biol. 2000 July; 10(7): 268-72
Hsu S K, Chiu C C, Dahms H U, Chou C K, Cheng C M, Chang W T, Cheng K C, Wang H D, Lin I L. Unfolded Protein Response (UPR) in Survival, Dormancy, Immunosuppression, Metastasis, and Treatments of Cancer Cells. Int J Mol Sci. 2019 May 22; 20(10):2518
Iconanu E L, Ciucurel M M, Georgescu L, and Ciucurel C1 Hand grip strength as a physical biomarker of aging from the perspective of a Fibonacci mathematical modeling. BMC Geriatr. 2018; 18: 2%.
Ishii T, Sakurai T, Usami H, Uchida K Oxidative modification of proteasome: identification of an oxidation-sensitive subunit in 26 S proteasome. Biochemistry, 2005 Oct. 25; 44(42): 13893-901.
Ishchenko Y, Carizales M G, Koleske A J. Regulation of the NMDA receptor by its cytoplasmic domains: (How) is the tail wagging the dog? Neuropharmacology. 2021 Sep. 1; 195:108634.
Jaisson S, Pietrement C, Gillery P Protein Carbamylation: Chemistry, Pathophysiological Involvement, and Biomarkers. Advances in Clinical Chemistry, Volume 84, 2018
Jian Z, Liang B, Pan X, Xu G, Guo S S, Li T, Zhou T, Xiao Y B, Li A L) CUEDC2 modulates cardiomyocyte oxidative capacity by regulating GPX1 stability. EMBO Mol Med. 2016 Jul. 1; 8(7):813-29.
Jones C L and Tepe J J Proteasome Activation to Combat Proteotoxicity. Molecules. 2019 August; 24(15): 2841.
Jung T, Grune T, The proteasome and the degradation of oxidized proteins: Part I-structure of proteasomes, Redox Biology 1(2013)178-182 Jung T, Höhn A, Grune T,
The proteasome and thedegradation of oxidized proteins: Part II—protein oxidation and proteasoma degradation. Redox Biology 2 (2014) 99-104
Jung T, Höhn A, Grune T. The proteasome and the degradation of oxidized proteins: Part III—Redox regulation of the proteasomal system. RedoxBiology 2 (2014) 388-394
Kapetanou M, Chondrogianni N, Petrakis S, Koliakos G, Gonos E S Proteasome activation enhances stemness and lifespan of human mesenchymal stem cells. Free Radical Biology and Medicine 103 (2017) 226-235
Kishi T, Iwata N. NMDA receptor antagonists interventions in schizophrenia: Metaanalysis of randomized, placebo-controlled trials. Journal of Psychiatric Research 47 (2013) 1143e1149
Kisselev, A. F. & Goldberg, A. L. Monitoring activity and inhibition of 26S proteasomes with fluorogenic peptide substrates. Methods Enzymol. 398, 364-378 (2005).
Kirkland J L, Tchkonia T Senolytic drugs: from discovery to translation. Journal of Intern med. 2020 November; 288(5):518-536
Klein A D, Ferreira N S, Ben-Dor S, Cox T M, Merrill A H, Futerman JAH. Identification of Modifier Genes in a Mouse Model of Gaucher Disease Cell Reports 2016, 16, 2546-2553
Kocaturk N M and Gozuacik D. Crosstalk Between Mammalian Autophagy and the Ubiquitin-Proteasome System. Frontiers in Cell and Developmental Biology 2018 Vol. 6 1Article 128
Korovila I, Hugo M, Castro J P, Weber D, Höhn A, Grune T, Jung T. Proteostasis, Oxidative Stress and Aging. Redox Biology 2017, 13, 550-567.
Kregel K C, Zhang H J An integrated view of oxidative stress in aging: basic mechanisms, functional effects, and pathological considerations, Am J Physiol Regul Integr Comp Physiol. 2007 January; 292(1):R18-36.
Lee B H, Lee M J, Park S, Oh D C, Elsasser S, Chen P C, Gartner C, Dimova N, Hanna J, Gygi S P, Wilson S M, King R W, Finley D. Enhancement of proteasome activity by a small-molecule inhibitor of USP14. Nature. 2010 Sep. 9; 467(7312):179-84.
Liu W, Jiang X, Zu Y, Yang Y, Liu Y, Sun X, Xu Z, Ding H, Zhao Q A comprehensive description of GluN2B-selective N-methyl-D aspartate (NMDA) receptor antagonists. European Journal of Medicinal Chemistry 200 (2020) 112447
Madden E, Logue S E, Healy S J, Manie S, Samali A. The role of the unfolded protein response in cancer progression: From oncogenesis to chemoresistance. Biol Cell. 2019 January; 111(1):1-17
Matsuda T, Cepko C L Electroporation and RNA interference in the rodent retina in vivo and in vitro.. Proc Natl Acad Sci USA. 2004 Jan. 6. 101(1):16-22.
McNaught K S, Olanow C V, Halliwell B, Isacson O, Jenner P. Failure of the ubiquitin-proteasome system in Parkinson's disease Nature reviews Neuroscience-September 2001 2(8):589-94
Menendez-Benito V, Verhoef L G, Masucci M G, Dantuma N P. Endoplasmic reticulum stress compromises the ubiquitin-proteasome system. Hum Mol Genet. 2005 Oct. 1. 14(19):2787-99.
Meyer T pLV-EF1a-IRES-Puro was a gift from Tobias Meyer (Addgene plasmid #85132; http://n2t.net/addgene:85132; RRID:Addgene_85132)
N'Diaye E N, Kajihara K K, Hsieh I, Morisaki H, Debnath J, Brown E J. PLIC proteins or ubiquilins regulate autophagy-dependent cell survival during nutrient starvation. EMBO Rep. 2009 February; 10(2):173-9.
Njomen E, Tepe J J Proteasome Activation as a New Therapeutic Approach To Target Proteotoxic Disorders J Med Chem. 2019 July 25; 62(14): 6469-6481.
Ohshima K, Nojima S, Tahara S, Kurashige M, Kawasaki K, Yumiko Hori Y, Taniguchi M,
Umakoshi Y, Okuzaki D, Wada N, Ikeda J I, Fukusaki E, Morii E. Serine racemase enhances growth of colorectal cancer by producing pyruvate from serine Nat Metab. 2020 January; 2(1):81-%.
Pan X C., Misrani, A., Tabassum, S, Yang L. Mitophagy pathways and Alzheimer's disease: From pathogenesis to treatment Mitochondrion Volume 59, July 2021, Pages 37-47
Paoletti P, Bellone C, Zhou Q NMDA receptor subunit diversity: impact on receptor properties, synaptic plasticity and disease. Nat Rev Neurosci. 2013 June; 14(6):383-400.
Pender S C, Smith A M, Finnoff J T, Huston J, and Stuart M J, Concussions in Ice Hockey—Moving Toward Objective Diagnoses and Point-of-care Treatment: A Review. Current Sports Medicine Reports 2020 September; 19(9):380-386
Petit-Pedrol M and Groc L Regulation of membrane NMDA receptors by dynamics and protein interactions J Cell Biol. 2021 Jan. 4; 220(1): e202006101.
Pickering A M, Davies K J. Degradation of damaged proteins: the main function of the 20S proteasome. Prog Mol Biol Transl Sci. 2012; 109:227-48..
Pohl C and Dikic I, Cellular quality control by the ubiquitin-proteasome system and autophagy Science 366, 818-822 (2019)
Rani K, Tyagi M, Mazumder M, Singh A, Shanmugam A, Accelerated identification of serine racemase inhibitor from Centella asiatica. Sci Rep. 2020 Mar. 13; 10(1):4640. doi: 10.1038/s41598-020-61494-1.
Raynes R, Pomatto L C D, Davies K J A Degradation of oxidized proteins by the proteasome: Distinguishing between the 20S, 26S,
and immunoproteasome proteolytic pathways Mol Aspects Med. 2016 August; 50:41-55.
Reichmann D, Voth W, Akob U. Maintaining a Healthy Proteome during Oxidative Stress Molecular Cell, Volume 69, Issue 2, 18 Jan. 2018, Pages 203-213
Roussel B D, Kruppa A J, Miranda E, Crowther D C, Lomas D A, Marciniak S J Endoplasmic reticulum dysfunction in neurological disease Lancet Neurol 2013; 12: 105-18
Saez-Atienzar S and Masliah E Cellular senescence and Alzheimer disease: the egg and the chicken scenario. 2020 Nat. Rev Neurosci. August; 21(8):433-444
Salomone S, Caraci F, Leggio G M, Fedotova J, Drago F New pharmacological strategies for treatment of Alzheimer's disease: focus on disease modifying drugs. Br J Clin Pharmacol. 2012 April; 73(4):504-17.
Sambrook and Russel eds., Molecular Cloning: A Laboratory Manual, 3nd Edition, Cold Spring Harbor Laboratory Press, Cold Spring Harbor 2001
Shafiei-Irannejad V, Abbaszadeh S, Janssen P M L, Soraya H. Memantine and its benefits for cancer, cardiovascular and neurological disorders. Eur J Pharmacol. 2021 Nov. 5; 910:174455.
Shalem-Cohavi N, Beery E., Nordenberg J, Rozovski U, Raanani P, Lahav M, Uziel O. The Effects of Proteasome Inhibitors on Telomerase Activity and Regulation in Multiple Myeloma Cells. Int J Mol Sci. 2019 May 21; 20(10):2509.
Shang F, Gong X, Palmer H J, Nowell T R, and Taylor A Age-related Decline in Ubiquitin Conjugation in Response to Oxidative Stress in the Lens. Exp. Eye Res. (1997) 64, 21-30
She J, Sheng R, Qin Z H Pharmacology and Potential Implications of Nicotinamide Adenine Dinucleotide Precursors. Aging and Disease; Volume 12, Number 8; 1879-1897, December 2021
Shi J, Knevel R, Suwannalai P, van der Linden M P, Janssen G M, van Veelen P A, Levarht N E, van der Helm-van Mil A H, Cerami A, Huizinga T W, Toes R E, L. A. Trouw L A. Autoantibodies recognizing carbamylated proteins are present in sera of patients with rheumatoid arthritis and predict joint damage. Proc. Natl. Acad. Sci. U.S.A 108 (2011) 17372-17377.
Siala O K, Pariseb E M, Pariseb L F, Gneccoa T, Bolaios-Guzmina C A, Ketamine: The final frontier or another depressing end?Behavioural Brain Research 383 (2020) 112508
Solis, G. M., Petrascheck, M. Measuring Caenorhabditis elegans Life Span in 96 Well Microtiter Plates. J. Vis. Exp. (2011). (49), e2496,
Sousa-Santos A R, Amaral T F. Differences in handgrip strength protocols to identify sarcopenia and frailty—a systematic review. BMC Geriatr. 2017; 17(1):238.
Tedeschi A, Dupraz S, Curcio M, Laskowski C J, Schaffran B, Flynn K C, Santos T E, Stern S, B J, Larson M J E, Gumiak C B, Witke W, Bradke F ADF/Cofilin-Mediated Actin Turnover Promotes Axon Regeneration in the Adult CNS. Neuron. 2019 September 25; 103(6): 1073-1085.
Thoppil H and Riabowol K Senolytics: A Translational Bridge Between Cellular Senescence and Organismal Aging. Frontiers in Cell and Developmental Biology January 2020|Volume 7|Article 367
Timilsina U, Ghimire D, Sharma S, Gaur R J Role of APOBEC3 proteins in proteasome inhibitor-mediated reactivation of latent HIV-1 viruses. Gen Virol. 2019 March; 100(3):523-532.
Trayssac M, Hannun Y A, and Obeid L M Role of sphingolipids in senescence: implication in aging and age-related diseases. J Clin Invest. 2018; 128(7):2702-2712.
Vieira M, Yong X L H, Roche K W, Anggono V. Regulation of NMDA glutamate receptor functions by the GluN2 subunits. J Neurochem. 2020 July; 154(2):121-143.
Vilchez D, Saez I and Dillin A., The role of protein clearance mechanisms in organismal ageing and age-related diseases. Nature Commun.. 2014 Dec. 8; 5:5659
Walther D M, Kasturi P, Zheng M, Pinkert S, Vecchi G, Ciryam P, Morimoto R I, Dobson C M, Vendruscolo M, Mann M, and F.-Ulrich Hartl F U Proteome Imbalance is Linked With Proteostasis Decline and Aggregation in Aging C. elegans. Cell. 2015 May 7; 161(4): 919-932.
Yoo D H, Im Y S, Jo E H, Kim B Y, Jo H Y, Park M H, Koo S K, Kim Y O Simple differentiation method using FBS identifies DUSP6 as a marker for fine-tuning of FGF-ERK signaling activity in human pluripotent stem cells. Biochem Biophys Res Commun. 2020 Jan. 8; 521(2):375-382.
Weng F L, He L Disrupted ubiquitin proteasome system underlying tau accumulation in Alzheimer's disease. Neurobiology of Aging 2021 March, 99:79-85
Yu H and Matouschek A. Recognition of Client Proteins by the Proteasome. Annu. Rev. Biophys. 2017.46:149-173.

1-51. (canceled)

52. Use of an N-methyl-D-aspartate receptor (NMDAR) antagonist to increase proteasome activity.

53. The use of an NMDAR antagonist according to claim 52, wherein:
a) the NMDAR antagonist increases the activity of the 20S proteasome directly or indirectly;
b) the NMDAR antagonist increases chymotrypsin-like, trypsin-like, and caspase-like activities, independently of the Ubiquitin-Proteasome System (UPS);
c) the NMDAR antagonist decreases the conversion of L-serine to D-serine by decreasing serine racemase;
d) the NMDAR antagonist enhances the degradation and turnover of actin.

54. The NMDAR antagonist for use according to claim 52, wherein the NMDAR antagonist is selected from the group consisting of memantine, ketamine, dextromethorphan, amantadine, ifenprodil, orphenadrine, or combinations thereof.

55. A composition comprising an NMDAR antagonist according to claim 52, for use in proteasome-meditated degradation of senescence-associated proteins including, p21, p53, ppRB and pAkt, thereby targeting cellular senescence, and reducing chronic low-grade inflammation linked to aging, for use as senolytic drug.

56. A composition comprising an NMDAR antagonist according to claim 52, for use in the prevention or treatment of diseases associated with impaired proteasome function.

57. A composition comprising an NMDAR antagonist according to claim 52, for use in enhancing healthspan and lifespan by increasing proteasome activity to upregulate telomerase activity, delaying cellular senescence, and enhancing the longevity and healthspan of an organism.

58. A composition comprising an NMDAR antagonist according to claim 52, for use in the prevention and treatment of aging-related conditions and diseases, wherein the aging-related conditions and diseases are selected from the group consisting of: misfolded and aggregated proteins, telomere attrition, telomere uncapping, advanced oxidation-glycation end-products, lipotoxicity, DNA damage, organelle dysfunction, stemness decline, cellular senescence, reduced muscle and skeletal function, impaired sensory perception, neurocognitive decline, cardiovascular diseases, cerebrovascular disease, type 2 diabetes, cancer, osteoarthritis, osteoporosis, hearing loss, skin aging and hair changes, age-related ocular pathologies including macular degeneration, cataracts, glaucoma, and diabetic retinopathy, acute and chronic kidney diseases, cerebral ischemia, traumatic brain injury, peripheral neuropathy, choroidal neovascularization, retinal ganglion cell loss, long-term effects of concussion, neurotoxic effects of astrocytes in Alzheimer's disease, and seizures.

59. A composition comprising an NMDAR antagonist according to claim 58, wherein the cancer is related to:
a) protein misfolding stress caused by the dysregulation of protein homeostasis; and
b) chemoresistance.

60. A composition comprising an NMDAR antagonist according to claim 52, for use in the prevention and treatment of a proteinopathy, wherein the proteinopathy is selected from the group consisting of: Alzheimer's disease, Parkinson's disease, Huntington's disease, Amyotrophic Lateral Sclerosis (ALS), Inclusion Body Myopathy, Systemic Amyloidosis, Autoimmune Diseases Associated with Proteinopathies.

61. The composition for use according to claim 60, wherein the autoimmune disease associated with proteinopathies is selected from the group consisting of: rheumatoid arthritis, systemic lupus erythematosus, thyroiditis, Hashimoto's thyroiditis, multiple sclerosis, myasthenia gravis, and type I diabetes.

62. A composition comprising an NMDAR antagonist according to claim 52, for use in the prevention and treatment of a psychiatric and neurological condition.

63. The composition for use according to claim 62, wherein the psychiatric and neurological condition is selected from the group consisting of: depression, schizophrenia, acute psychosis, anxiety, insomnia, confusion, migraine, and neurodegenerative diseases.

64. The composition according to claim 52, wherein:
a) the composition is a pharmaceutical composition;
b) the composition is a cosmetic composition;
c) the composition is a pharmaceutical composition comprising memantine in a concentration that ranges from 0.01 mg/kg to 10 mg/kg body weight, for treating Alzheimer's disease with recommended administration intervals of 6-8 hours.

65. The composition according to claim 52, wherein the NMDAR antagonist is memantine and the administration is oral, rectal, transmucosal, parenteral, or in a sustained-release formulation.
